SEQUENCE LISTING (94 sequences)

SEQ ID NO: 1 - protein, 119 aa, Artificial Sequence
  G16 VH
QVQLVQSGAEVTKPGASVKVSCRASGYPFSNFFMNWVRQAPGQGLEWMGIINPIGGSTTYAQKFQGRVTMTRDTSTDTVYMELSSLRSDDTAVYYCARSSSHLGAFDIWGQGTMVTVSS

SEQ ID NO: 2 - protein, 108 aa, Artificial Sequence
  G16 VL
DIQLTQSPSSVSASVGDRVTITCRASQSISTWVAWYQHKPGVAPKLLIYASPTLQSGVPSRFSGSGSGTDFTLTISSLQSEDFATYYCQQAHSFPITFGQGTRLEIKR

SEQ ID NO: 3 - protein, 5 aa, Artificial Sequence
  Kabat G16 CDR-H1
NFFMN

SEQ ID NO: 4 - protein, 17 aa, Artificial Sequence
  Kabat G16 CDR-H2
IINPIGGSTTYAQKFQG

SEQ ID NO: 5 - protein, 10 aa, Artificial Sequence
  Kabat/Chothia G16 CDR-H3
SSSHLGAFDI

SEQ ID NO: 6 - protein, 11 aa, Artificial Sequence
  Kabat/Chothia G16 CDR-L1
RASQSISTWVA

SEQ ID NO: 7 - protein, 7 aa, Artificial Sequence
  Kabat/Chothia G16 CDR-L2
ASPTLQS

SEQ ID NO: 8 - protein, 9 aa, Artificial Sequence
  Kabat/IMGT/Chothia G16 CDR-L3
QQAHSFPIT

SEQ ID NO: 9 - protein, 8 aa, Artificial Sequence
  IMGT G16 CDR-H1
GYPFSNFF

SEQ ID NO: 10 - protein, 8 aa, Artificial Sequence
  IMGT G16 CDR-H2
INPIGGST

SEQ ID NO: 11 - protein, 12 aa, Artificial Sequence
  IMGT G16 CDR-H3
ARSSSHLGAFDI

SEQ ID NO: 12 - protein, 6 aa, Artificial Sequence
  IMGT G16 CDR-L1
QSISTW

SEQ ID NO: 13 - protein, 3 aa, Artificial Sequence
  IMGT G16 CDR-L2
ASP

SEQ ID NO: 14 - protein, 7 aa, Artificial Sequence
  Chothia  G16 CDR-H1
GYPFSNF

SEQ ID NO: 15 - protein, 6 aa, Artificial Sequence
  Chothia  G16 CDR-H2
NPIGGS

SEQ ID NO: 16 - protein, 121 aa, Artificial Sequence
  G5 VH
QVQLVQSGGGLVQPGRSLRLSCAASGFTFDDYAMHWVRQAPGKGLEWVSGISWNSGSIGYADSVKGRFTISRDNAKNSLYLQMNSLRAEDTALYYCAKDMGSSGWSPVDYWGQGTLVTVSS

SEQ ID NO: 17 - protein, 110 aa, Artificial Sequence
  G5 VL
DIQVTQSPSSLSASVGDRVTITCRASQGINHYLAWYQQKPGKAPKLLIYGASTLQTGVSSRFSGSGSGTEFTLTISSLQPEDFATYYCQQLNGYPLGYTFGQGTKVEIKR

SEQ ID NO: 18 - protein, 5 aa, Artificial Sequence
  Kabat G5 CDR-H1
DYAMH

SEQ ID NO: 19 - protein, 17 aa, Artificial Sequence
  Kabat G5 CDR-H2
GISWNSGSIGYADSVKG

SEQ ID NO: 20 - protein, 12 aa, Artificial Sequence
  Kabat/Chothia G5 CDR-H3
DMGSSGWSPVDY

SEQ ID NO: 21 - protein, 11 aa, Artificial Sequence
  Kabat/Chothia G5 CDR-L1
RASQGINHYLA

SEQ ID NO: 22 - protein, 7 aa, Artificial Sequence
  Kabat/Chothia G5 CDR-L2
GASTLQT

SEQ ID NO: 23 - protein, 11 aa, Artificial Sequence
  Kabat/IMGT/Chothia G5 CDR-L3
QQLNGYPLGYT

SEQ ID NO: 24 - protein, 8 aa, Artificial Sequence
  IMGT G5 CDR-H1
GFTFDDYA

SEQ ID NO: 25 - protein, 8 aa, Artificial Sequence
  IMGT G5 CDR-H2
ISWNSGSI

SEQ ID NO: 26 - protein, 14 aa, Artificial Sequence
  IMGT G5 CDR-H3
AKDMGSSGWSPVDY

SEQ ID NO: 27 - protein, 6 aa, Artificial Sequence
  IMGT G5 CDR-L1
QGINHY

SEQ ID NO: 28 - protein, 3 aa, Artificial Sequence
  IMGT G5 CDR-L2
GAS

SEQ ID NO: 29 - protein, 7 aa, Artificial Sequence
  Chothia G5 CDR-H1
GFTFDDY

SEQ ID NO: 30 - protein, 6 aa, Artificial Sequence
  Chothia G5 CDR-H2
SWNSGS

SEQ ID NO: 31 - protein, 121 aa, Artificial Sequence
  G9 VH
EVQLVETGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGYWATAAFDIWGQGTPVTVSS

SEQ ID NO: 32 - protein, 111 aa, Artificial Sequence
  G9 VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPTWTFGQGTKVEIKR

SEQ ID NO: 33 - protein, 5 aa, Artificial Sequence
  Kabat G9 CDR-H1
SYAMS

SEQ ID NO: 34 - protein, 17 aa, Artificial Sequence
  Kabat G9 CDR-H2
AISGSGGSTYYADSVKG

SEQ ID NO: 35 - protein, 12 aa, Artificial Sequence
  Kabat/Chothia  G9 CDR-H3
GSGYWATAAFDI

SEQ ID NO: 36 - protein, 12 aa, Artificial Sequence
  Kabat/Chothia G9 CDR-L1
RASQSVSSSYLA

SEQ ID NO: 37 - protein, 7 aa, Artificial Sequence
  Kabat/Chothia G9 CDR-L2
GASSRAT

SEQ ID NO: 38 - protein, 11 aa, Artificial Sequence
  Kabat/IMGT/Chothia G9 CDR-L3
QQYGSSPPTWT

SEQ ID NO: 39 - protein, 8 aa, Artificial Sequence
  IMGT G9 CDR-H1
GFTFSSYA

SEQ ID NO: 40 - protein, 8 aa, Artificial Sequence
  IMGT G9 CDR-H2
ISGSGGST

SEQ ID NO: 41 - protein, 14 aa, Artificial Sequence
  IMGT G9 CDR-H3
AKGSGYWATAAFDI

SEQ ID NO: 42 - protein, 7 aa, Artificial Sequence
  IMGT G9 CDR-L1
QSVSSSY

SEQ ID NO: 43 - protein, 3 aa, Artificial Sequence
  IMGT G9 CDR-L2
GAS

SEQ ID NO: 44 - protein, 7 aa, Artificial Sequence
  Chothia G9 CDR-H1
GFTFSSY

SEQ ID NO: 45 - protein, 6 aa, Artificial Sequence
  Chothia G9 CDR-H2
SGSGGS

SEQ ID NO: 46 - DNA, 357 bp, Artificial Sequence
  nucleotide sequence of G16 VH
caggtgcagctggtgcagagtggtgccgaggtgaccaagcctggcgcttccgtgaaggtgtcatgccgtgcttccggctatcccttcagcaacttctttatgaactgggtgcgccaggccccgggacaaggtctcgagtggatgggcatcatcaatccaatcggtggctccacgacctacgcgcagaagttccagggacgcgtcactatgacccgcgacacttccaccgacacggtctacatggagctgtcatctctgcgcagcgatgacaccgccgtgtactactgtgctcggtctagcagccacctgggcgccttcgacatatggggccagggcactatggtgaccgtgtcctcc

SEQ ID NO: 47 - DNA, 324 bp, Artificial Sequence
  nucleotide sequence of G16 VL
gacatccagctgacccagagtccctcttccgtgtccgctagcgtgggggacagggttaccatcacctgccgagcctcccaaagcatctctacctgggtcgcgtggtaccagcacaaaccgggagtagcccccaagctgctcatttacgcatccccaaccctgcagagcggtgtcccgtccagattttcagggtcgggctctgggacagatttcaccctcactatttcgtcgcttcagagcgaagactttgccacctattactgtcagcaggcgcattccttccccatcaccttcggccagggcactcgcctggagatcaagcgc

SEQ ID NO: 48 - DNA, 363 bp, Artificial Sequence
  nucleotide sequence of G5 VH
caggtgcagctggtgcaatctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatatgggtagcagtggctggtcccccgttgactactggggccagggaaccctggtcaccgtctcctca

SEQ ID NO: 49 - DNA, 330 bp, Artificial Sequence
  nucleotide sequence of G5 VL
gacatccaggtgacccagtctccatcctccctgtctgcatctgttggagacagagtcaccatcacttgtcgggccagccagggcattaaccattatttagcctggtatcaacaaaagccagggaaagcccctaagctcctgatctacggtgcctccactttgcaaactggggtctcatcgcggttcagcggcagtggatctgggacagaattcactctcaccatcagcagcctgcagcctgaagattttgcaacttattactgtcaacagcttaacggttaccctctggggtacacttttggccaggggaccaaggtggaaatcaaacgt

SEQ ID NO: 50 - DNA, 363 bp, Artificial Sequence
  nucleotide sequence of G9 VH
gaggtgcagctggtggagaccgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaggtagtggttattgggcaacggccgcttttgatatctggggccagggaaccccggtcaccgtctcttca

SEQ ID NO: 51 - DNA, 333 bp, Artificial Sequence
  nucleotide sequence of G9 VL
gaaattgtgttgacacagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcccacgtggacgttcggccaagggaccaaggtggagatcaaacgt

SEQ ID NO: 52 - protein, 17 aa, Artificial Sequence
  amino acid sequence of Linker
ASGGGGSGGRASGGGGS

SEQ ID NO: 53 - DNA, 51 bp, Artificial Sequence
  nucleotide sequence of Linker
gcttctggcggcggcgggtccgggggccgcgcatctggcggcggtggttcc

SEQ ID NO: 54 - protein, 244 aa, Artificial Sequence
  amino acid sequence of G16-scFv
QVQLVQSGAEVTKPGASVKVSCRASGYPFSNFFMNWVRQAPGQGLEWMGIINPIGGSTTYAQKFQGRVTMTRDTSTDTVYMELSSLRSDDTAVYYCARSSSHLGAFDIWGQGTMVTVSSASGGGGSGGRASGGGGSDIQLTQSPSSVSASVGDRVTITCRASQSISTWVAWYQHKPGVAPKLLIYASPTLQSGVPSRFSGSGSGTDFTLTISSLQSEDFATYYCQQAHSFPITFGQGTRLEIKR

SEQ ID NO: 55 - DNA, 732 bp, Artificial Sequence
  nucleotide sequence of G16-scFv
caggtgcagctggtgcagagtggtgccgaggtgaccaagcctggcgcttccgtgaaggtgtcatgccgtgcttccggctatcccttcagcaacttctttatgaactgggtgcgccaggccccgggacaaggtctcgagtggatgggcatcatcaatccaatcggtggctccacgacctacgcgcagaagttccagggacgcgtcactatgacccgcgacacttccaccgacacggtctacatggagctgtcatctctgcgcagcgatgacaccgccgtgtactactgtgctcggtctagcagccacctgggcgccttcgacatatggggccagggcactatggtgaccgtgtcctccgcttctggcggcggcgggtccgggggccgcgcatctggcggcggtggttccgacatccagctgacccagagtccctcttccgtgtccgctagcgtgggggacagggttaccatcacctgccgagcctcccaaagcatctctacctgggtcgcgtggtaccagcacaaaccgggagtagcccccaagctgctcatttacgcatccccaaccctgcagagcggtgtcccgtccagattttcagggtcgggctctgggacagatttcaccctcactatttcgtcgcttcagagcgaagactttgccacctattactgtcagcaggcgcattccttccccatcaccttcggccagggcactcgcctggagatcaagcgc

SEQ ID NO: 56 - protein, 248 aa, Artificial Sequence
  amino acid sequence of G5-scFv
QVQLVQSGGGLVQPGRSLRLSCAASGFTFDDYAMHWVRQAPGKGLEWVSGISWNSGSIGYADSVKGRFTISRDNAKNSLYLQMNSLRAEDTALYYCAKDMGSSGWSPVDYWGQGTLVTVSSASGGGGSGGRASGGGGSDIQVTQSPSSLSASVGDRVTITCRASQGINHYLAWYQQKPGKAPKLLIYGASTLQTGVSSRFSGSGSGTEFTLTISSLQPEDFATYYCQQLNGYPLGYTFGQGTKVEIKR

SEQ ID NO: 57 - DNA, 744 bp, Artificial Sequence
  nucleotide sequence of G5-scFv
caggtgcagctggtgcaatctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatatgggtagcagtggctggtcccccgttgactactggggccagggaaccctggtcaccgtctcctcagctagcggaggtggcggttctggcgggcgcgcctcaggtggaggtgggtccgacatccaggtgacccagtctccatcctccctgtctgcatctgttggagacagagtcaccatcacttgtcgggccagccagggcattaaccattatttagcctggtatcaacaaaagccagggaaagcccctaagctcctgatctacggtgcctccactttgcaaactggggtctcatcgcggttcagcggcagtggatctgggacagaattcactctcaccatcagcagcctgcagcctgaagattttgcaacttattactgtcaacagcttaacggttaccctctggggtacacttttggccaggggaccaaggtggaaatcaaacgt

SEQ ID NO: 58 - protein, 249 aa, Artificial Sequence
  amino acid sequence of G9-scFv
EVQLVETGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGYWATAAFDIWGQGTPVTVSSASGGGGSGGRASGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPTWTFGQGTKVEIKR

SEQ ID NO: 59 - DNA, 747 bp, Artificial Sequence
  nucleotide sequence of G9-scFv
gaggtgcagctggtggagaccgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaggtagtggttattgggcaacggccgcttttgatatctggggccagggaaccccggtcaccgtctcttcagctagcggaggtggcggttctggcgggcgcgcctcaggtggaggtgggtccgaaattgtgttgacacagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcccacgtggacgttcggccaagggaccaaggtggagatcaaacgt

SEQ ID NO: 60 - protein, 21 aa, Artificial Sequence
  amino acid sequence of Signal peptide 1
MALPVTALLLPLALLLHAARP

SEQ ID NO: 61 - DNA, 63 bp, Artificial Sequence
  nucleotide sequence of Signal peptide 1
atggccttgcctgttacagccttgttactgcccctggcgctgcttctacacgccgctcgtcct

SEQ ID NO: 62 - protein, 45 aa, Artificial Sequence
  amino acid sequence of CD8 hinge region
TTTPAPRPPTPAPTIASQPLSLRPEACRPAAGGAVHTRGLDFACD

SEQ ID NO: 63 - DNA, 135 bp, Artificial Sequence
  nucleotide sequence of CD8 hinge region
accacgacgccagcgccgcgaccaccaacaccggcgcccaccatcgcgtcgcagcccctgtccctgcgcccagaagcgtgccggccagcggcggggggcgcagtgcacacgagggggctggacttcgcctgtgat

SEQ ID NO: 64 - protein, 24 aa, Artificial Sequence
  amino acid sequence of CD8 transmembrane region
IYIWAPLAGTCGVLLLSLVITLYC

SEQ ID NO: 65 - DNA, 72 bp, Artificial Sequence
  nucleotide sequence of CD8 transmembrane region
atctacatttgggcccctctggctggtacttgcggggtcctgctgctttcactcgtgatcactctttactgt

SEQ ID NO: 66 - protein, 42 aa, Artificial Sequence
  amino acid sequence of Intracellular signaling domain (4-1BB)
KRGRKKLLYIFKQPFMRPVQTTQEEDGCSCRFPEEEEGGCEL

SEQ ID NO: 67 - DNA, 126 bp, Artificial Sequence
  nucleotide sequence of Intracellular signaling domain (4-1BB)
aagcgcggtcggaagaagctgctgtacatctttaagcaacccttcatgaggcctgtgcagactactcaagaggaggacggctgttcatgccggttcccagaggaggaggaaggcggctgcgaactg

SEQ ID NO: 68 - protein, 112 aa, Artificial Sequence
  amino acid sequence of Intracellular signaling domain (CD3zeta)
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - DNA, 336 bp, Artificial Sequence
  nucleotide sequence of Intracellular signaling domain (CD3zeta)
cgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 70 - protein, 154 aa, Artificial Sequence
  amino acid sequence of Intracellular signaling domain (4-1BB-CD3zeta)
KRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - DNA, 462 bp, Artificial Sequence
  nucleotide sequence of Intracellular signaling domain (4-1BB-CD3zeta)
aagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 72 - protein, 26 aa, Artificial Sequence
  amino acid sequence of P2A
RAKRGSGATNFSLLKQAGDVEENPGP

SEQ ID NO: 73 - DNA, 78 bp, Artificial Sequence
  nucleotide sequence-1 of P2A
cgggccaagagaggatctggagccacaaattttagcctgctgaaacaggccggagatgtggaggaaaaccctggccct

SEQ ID NO: 74 - protein, 20 aa, Artificial Sequence
  amino acid sequence of Signal peptide 2
MYRMQLLSCIALSLALVTNS

SEQ ID NO: 75 - DNA, 60 bp, Artificial Sequence
  nucleotide sequence of Signal peptide 2
atgtataggatgcagctgctgagctgtattgccctgagccttgcactggtgaccaatagc

SEQ ID NO: 76 - DNA, 78 bp, Artificial Sequence
  nucleotide sequence-2 of P2A
cgagctaaacgaggctcaggcgcgacgaactttagtttgctgaagcaagctggggatgtagaggaaaatccgggtccc

SEQ ID NO: 77 - protein, 238 aa, Artificial Sequence
  amino acid sequence of PD1-scFv
EVQLVQSGAEVKKPGASVKVSCKASGYTFTDYYMNWVREAPGQGLEWMGEINPKYGDTTYNQKFKDRATLTVDKSTSTAYMELSSLRSDDTAVYYCAKGIRLFDSWGQGTLVTVSSGGGGSGGGGSGGGGSDIQLTQSPSSLSASVGDRVTITCRPSGNVHNYFAWYQQKPGKSPKLLVYNAKTLAEGVPSRFSGSGSGTDYTLTISSLQPEDFATYYCHHYYTAPFTFGGGTKVEIK

SEQ ID NO: 78 - DNA, 714 bp, Artificial Sequence
  nucleotide sequence of PD1-scFv
gaggtgcagctggtgcagagcggcgccgaggtgaagaagcctggcgccagcgtgaaggtgagctgcaaggcctccggctacaccttcaccgactactacatgaactgggtgcgagaggcccccggccagggcctggagtggatgggcgagatcaatcccaagtacggcgataccacctacaatcagaagttcaaggaccgggccaccctgaccgtggataagtctacatctacagcctatatggagctgagcagcctgagatccgatgataccgcagtgtattattgtgccaaaggaattagactgtttgactcttggggccagggaaccctagtgaccgtaagcagcggaggaggaggcagcggaggaggcggatctggcggcggcggctccgacatccagctgacccagagccccagcagcctgagcgccagcgtgggcgacagggtgaccatcacctgccggccctccggcaacgtgcacaattacttcgcctggtaccagcagaagcccggcaagagccctaagctgctggtgtataatgccaagacactggctgaaggcgtgcctagtaggtttagcggcagcggaagcggcactgattatacactgacaatttctagcctgcagcctgaggattttgctacatattattgtcatcattactatacagccccttttacatttggaggcggaacaaaggtggagattaag

SEQ ID NO: 79 - protein, 116 aa, Artificial Sequence
  PD1-scFv VH
EVQLVQSGAEVKKPGASVKVSCKASGYTFTDYYMNWVREAPGQGLEWMGEINPKYGDTTYNQKFKDRATLTVDKSTSTAYMELSSLRSDDTAVYYCAKGIRLFDSWGQGTLVTVSS

SEQ ID NO: 80 - protein, 107 aa, Artificial Sequence
  PD1-scFv VL
DIQLTQSPSSLSASVGDRVTITCRPSGNVHNYFAWYQQKPGKSPKLLVYNAKTLAEGVPSRFSGSGSGTDYTLTISSLQPEDFATYYCHHYYTAPFTFGGGTKVEIK

SEQ ID NO: 81 - protein, 395 aa, Artificial Sequence [sequence omitted]

SEQ ID NO: 82 - DNA, 1185 bp, Artificial Sequence
  nucleotide sequence of mIL15
atggattggacttggattttgttcctcgttgccgcagcgactcgcgtccatagtaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttccgggggcggcagtggaggtggcggtagcggcgggggtggctctggtggaggcggctctgggggcggaagtctgcagatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccaccta

SEQ ID NO: 83 - protein, 488 aa, Artificial Sequence
  amino acid sequence of G16-CAR
MALPVTALLLPLALLLHAARPQVQLVQSGAEVTKPGASVKVSCRASGYPFSNFFMNWVRQAPGQGLEWMGIINPIGGSTTYAQKFQGRVTMTRDTSTDTVYMELSSLRSDDTAVYYCARSSSHLGAFDIWGQGTMVTVSSASGGGGSGGRASGGGGSDIQLTQSPSSVSASVGDRVTITCRASQSISTWVAWYQHKPGVAPKLLIYASPTLQSGVPSRFSGSGSGTDFTLTISSLQSEDFATYYCQQAHSFPITFGQGTRLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - DNA, 1464 bp, Artificial Sequence
  nucleotide sequence of G16 CAR
atggccttgcctgttacagccttgttactgcccctggcgctgcttctacacgccgctcgtcctcaggtgcagctggtgcagagtggtgccgaggtgaccaagcctggcgcttccgtgaaggtgtcatgccgtgcttccggctatcccttcagcaacttctttatgaactgggtgcgccaggccccgggacaaggtctcgagtggatgggcatcatcaatccaatcggtggctccacgacctacgcgcagaagttccagggacgcgtcactatgacccgcgacacttccaccgacacggtctacatggagctgtcatctctgcgcagcgatgacaccgccgtgtactactgtgctcggtctagcagccacctgggcgccttcgacatatggggccagggcactatggtgaccgtgtcctccgcttctggcggcggcgggtccgggggccgcgcatctggcggcggtggttccgacatccagctgacccagagtccctcttccgtgtccgctagcgtgggggacagggttaccatcacctgccgagcctcccaaagcatctctacctgggtcgcgtggtaccagcacaaaccgggagtagcccccaagctgctcatttacgcatccccaaccctgcagagcggtgtcccgtccagattttcagggtcgggctctgggacagatttcaccctcactatttcgtcgcttcagagcgaagactttgccacctattactgtcagcaggcgcattccttccccatcaccttcggccagggcactcgcctggagatcaagcgcaccacgacgccagcgccgcgaccaccaacaccggcgcccaccatcgcgtcgcagcccctgtccctgcgcccagaagcgtgccggccagcggcggggggcgcagtgcacacgagggggctggacttcgcctgtgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 85 - DNA, 2316 bp, Artificial Sequence
  nucleotide sequence of G16-PD1 CAR
atggccttgcctgttacagccttgttactgcccctggcgctgcttctacacgccgctcgtcctcaggtgcagctggtgcagagtggtgccgaggtgaccaagcctggcgcttccgtgaaggtgtcatgccgtgcttccggctatcccttcagcaacttctttatgaactgggtgcgccaggccccgggacaaggtctcgagtggatgggcatcatcaatccaatcggtggctccacgacctacgcgcagaagttccagggacgcgtcactatgacccgcgacacttccaccgacacggtctacatggagctgtcatctctgcgcagcgatgacaccgccgtgtactactgtgctcggtctagcagccacctgggcgccttcgacatatggggccagggcactatggtgaccgtgtcctccgcttctggcggcggcgggtccgggggccgcgcatctggcggcggtggttccgacatccagctgacccagagtccctcttccgtgtccgctagcgtgggggacagggttaccatcacctgccgagcctcccaaagcatctctacctgggtcgcgtggtaccagcacaaaccgggagtagcccccaagctgctcatttacgcatccccaaccctgcagagcggtgtcccgtccagattttcagggtcgggctctgggacagatttcaccctcactatttcgtcgcttcagagcgaagactttgccacctattactgtcagcaggcgcattccttccccatcaccttcggccagggcactcgcctggagatcaagcgcaccacgacgccagcgccgcgaccaccaacaccggcgcccaccatcgcgtcgcagcccctgtccctgcgcccagaagcgtgccggccagcggcggggggcgcagtgcacacgagggggctggacttcgcctgtgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggcgggccaagagaggatctggagccacaaattttagcctgctgaaacaggccggagatgtggaggaaaaccctggccctatgtataggatgcagctgctgagctgtattgccctgagccttgcactggtgaccaatagcgaggtgcagctggtgcagagcggcgccgaggtgaagaagcctggcgccagcgtgaaggtgagctgcaaggcctccggctacaccttcaccgactactacatgaactgggtgcgagaggcccccggccagggcctggagtggatgggcgagatcaatcccaagtacggcgataccacctacaatcagaagttcaaggaccgggccaccctgaccgtggataagtctacatctacagcctatatggagctgagcagcctgagatccgatgataccgcagtgtattattgtgccaaaggaattagactgtttgactcttggggccagggaaccctagtgaccgtaagcagcggaggaggaggcagcggaggaggcggatctggcggcggcggctccgacatccagctgacccagagccccagcagcctgagcgccagcgtgggcgacagggtgaccatcacctgccggccctccggcaacgtgcacaattacttcgcctggtaccagcagaagcccggcaagagccctaagctgctggtgtataatgccaagacactggctgaaggcgtgcctagtaggtttagcggcagcggaagcggcactgattatacactgacaatttctagcctgcagcctgaggattttgctacatattattgtcatcattactatacagccccttttacatttggaggcggaacaaaggtggagattaag

SEQ ID NO: 86 - DNA, 2727 bp, Artificial Sequence
  nucleotide sequence of G16-mIL15 CAR
atggccttgcctgttacagccttgttactgcccctggcgctgcttctacacgccgctcgtcctcaggtgcagctggtgcagagtggtgccgaggtgaccaagcctggcgcttccgtgaaggtgtcatgccgtgcttccggctatcccttcagcaacttctttatgaactgggtgcgccaggccccgggacaaggtctcgagtggatgggcatcatcaatccaatcggtggctccacgacctacgcgcagaagttccagggacgcgtcactatgacccgcgacacttccaccgacacggtctacatggagctgtcatctctgcgcagcgatgacaccgccgtgtactactgtgctcggtctagcagccacctgggcgccttcgacatatggggccagggcactatggtgaccgtgtcctccgcttctggcggcggcgggtccgggggccgcgcatctggcggcggtggttccgacatccagctgacccagagtccctcttccgtgtccgctagcgtgggggacagggttaccatcacctgccgagcctcccaaagcatctctacctgggtcgcgtggtaccagcacaaaccgggagtagcccccaagctgctcatttacgcatccccaaccctgcagagcggtgtcccgtccagattttcagggtcgggctctgggacagatttcaccctcactatttcgtcgcttcagagcgaagactttgccacctattactgtcagcaggcgcattccttccccatcaccttcggccagggcactcgcctggagatcaagcgcaccacgacgccagcgccgcgaccaccaacaccggcgcccaccatcgcgtcgcagcccctgtccctgcgcccagaagcgtgccggccagcggcggggggcgcagtgcacacgagggggctggacttcgcctgtgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggcgagctaaacgaggctcaggcgcgacgaactttagtttgctgaagcaagctggggatgtagaggaaaatccgggtcccatggattggacttggattttgttcctcgttgccgcagcgactcgcgtccatagtaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttccgggggcggcagtggaggtggcggtagcggcgggggtggctctggtggaggcggctctgggggcggaagtctgcagatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccaccta

SEQ ID NO: 87 - DNA, 3579 bp, Artificial Sequence
  nucleotide sequence of G16-PD1-mIL15 CAR
atggccttgcctgttacagccttgttactgcccctggcgctgcttctacacgccgctcgtcctcaggtgcagctggtgcagagtggtgccgaggtgaccaagcctggcgcttccgtgaaggtgtcatgccgtgcttccggctatcccttcagcaacttctttatgaactgggtgcgccaggccccgggacaaggtctcgagtggatgggcatcatcaatccaatcggtggctccacgacctacgcgcagaagttccagggacgcgtcactatgacccgcgacacttccaccgacacggtctacatggagctgtcatctctgcgcagcgatgacaccgccgtgtactactgtgctcggtctagcagccacctgggcgccttcgacatatggggccagggcactatggtgaccgtgtcctccgcttctggcggcggcgggtccgggggccgcgcatctggcggcggtggttccgacatccagctgacccagagtccctcttccgtgtccgctagcgtgggggacagggttaccatcacctgccgagcctcccaaagcatctctacctgggtcgcgtggtaccagcacaaaccgggagtagcccccaagctgctcatttacgcatccccaaccctgcagagcggtgtcccgtccagattttcagggtcgggctctgggacagatttcaccctcactatttcgtcgcttcagagcgaagactttgccacctattactgtcagcaggcgcattccttccccatcaccttcggccagggcactcgcctggagatcaagcgcaccacgacgccagcgccgcgaccaccaacaccggcgcccaccatcgcgtcgcagcccctgtccctgcgcccagaagcgtgccggccagcggcggggggcgcagtgcacacgagggggctggacttcgcctgtgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggcgggccaagagaggatctggagccacaaattttagcctgctgaaacaggccggagatgtggaggaaaaccctggccctatgtataggatgcagctgctgagctgtattgccctgagccttgcactggtgaccaatagcgaggtgcagctggtgcagagcggcgccgaggtgaagaagcctggcgccagcgtgaaggtgagctgcaaggcctccggctacaccttcaccgactactacatgaactgggtgcgagaggcccccggccagggcctggagtggatgggcgagatcaatcccaagtacggcgataccacctacaatcagaagttcaaggaccgggccaccctgaccgtggataagtctacatctacagcctatatggagctgagcagcctgagatccgatgataccgcagtgtattattgtgccaaaggaattagactgtttgactcttggggccagggaaccctagtgaccgtaagcagcggaggaggaggcagcggaggaggcggatctggcggcggcggctccgacatccagctgacccagagccccagcagcctgagcgccagcgtgggcgacagggtgaccatcacctgccggccctccggcaacgtgcacaattacttcgcctggtaccagcagaagcccggcaagagccctaagctgctggtgtataatgccaagacactggctgaaggcgtgcctagtaggtttagcggcagcggaagcggcactgattatacactgacaatttctagcctgcagcctgaggattttgctacatattattgtcatcattactatacagccccttttacatttggaggcggaacaaaggtggagattaagcgagctaaacgaggctcaggcgcgacgaactttagtttgctgaagcaagctggggatgtagaggaaaatccgggtcccatggattggacttggattttgttcctcgttgccgcagcgactcgcgtccatagtaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttccgggggcggcagtggaggtggcggtagcggcgggggtggctctggtggaggcggctctgggggcggaagtctgcagatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccaccta

SEQ ID NO: 88 - protein, 492 aa, Artificial Sequence
  amino acid sequence of G5-CAR
MALPVTALLLPLALLLHAARPQVQLVQSGGGLVQPGRSLRLSCAASGFTFDDYAMHWVRQAPGKGLEWVSGISWNSGSIGYADSVKGRFTISRDNAKNSLYLQMNSLRAEDTALYYCAKDMGSSGWSPVDYWGQGTLVTVSSASGGGGSGGRASGGGGSDIQVTQSPSSLSASVGDRVTITCRASQGINHYLAWYQQKPGKAPKLLIYGASTLQTGVSSRFSGSGSGTEFTLTISSLQPEDFATYYCQQLNGYPLGYTFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - DNA, 1476 bp, Artificial Sequence
  nucleotide sequence of G5 CAR
atggccttaccagtgaccgccttgctcctgccgctggccttgctgctccacgccgccaggccgcaggtgcagctggtgcaatctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatatgggtagcagtggctggtcccccgttgactactggggccagggaaccctggtcaccgtctcctcagctagcggaggtggcggttctggcgggcgcgcctcaggtggaggtgggtccgacatccaggtgacccagtctccatcctccctgtctgcatctgttggagacagagtcaccatcacttgtcgggccagccagggcattaaccattatttagcctggtatcaacaaaagccagggaaagcccctaagctcctgatctacggtgcctccactttgcaaactggggtctcatcgcggttcagcggcagtggatctgggacagaattcactctcaccatcagcagcctgcagcctgaagattttgcaacttattactgtcaacagcttaacggttaccctctggggtacacttttggccaggggaccaaggtggaaatcaaacgtaccacgacgccagcgccgcgaccaccaacaccggcgcccaccatcgcgtcgcagcccctgtccctgcgcccagaagcgtgccggccagcggcggggggcgcagtgcacacgagggggctggacttcgcctgtgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 90 - protein, 493 aa, Artificial Sequence
  amino acid sequence of G9-CAR
MALPVTALLLPLALLLHAARPEVQLVETGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGYWATAAFDIWGQGTPVTVSSASGGGGSGGRASGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPTWTFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - DNA, 1479 bp, Artificial Sequence
  nucleotide sequence of G9 CAR
atggccttaccagtgaccgccttgctcctgccgctggccttgctgctccacgccgccaggccggaggtgcagctggtggagaccgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaggtagtggttattgggcaacggccgcttttgatatctggggccagggaaccccggtcaccgtctcttcagctagcggaggtggcggttctggcgggcgcgcctcaggtggaggtgggtccgaaattgtgttgacacagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcccacgtggacgttcggccaagggaccaaggtggagatcaaacgtaccacgacgccagcgccgcgaccaccaacaccggcgcccaccatcgcgtcgcagcccctgtccctgcgcccagaagcgtgccggccagcggcggggggcgcagtgcacacgagggggctggacttcgcctgtgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 92 - DNA, 20 bp, Artificial Sequence
  Primer PKLT1F
gcttccggctcgtatgttgt

SEQ ID NO: 93 - DNA, 20 bp, Artificial Sequence
  Primer PKLT1R
gacaggaggttgaggcaggt

SEQ ID NO: 94 - protein, 233 aa, Artificial Sequence
  Fc (human IgG1)
PEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0269026 A1
(43) Pub. Date: Aug. 28, 2025

(54) CHIMERIC ANTIGEN RECEPTOR WHICH SPECIFICALLY BINDS TO MSLN, AND APPLICATION THEREOF

(71) Applicant: CHENGDU KELUN PRECISION BIOTECHNOLOGY CO., LTD., Chengdu, Sichuan (CN)

(72) Inventors: Jianhui Chang, Chengdu (CN); Yanlin Zhu, Chengdu (CN); Ziming Wu, Chengdu (CN); Zhenzhen Cai, Chengdu (CN); Hong Li, CRANBURY, NJ (US); Zhou Lu, Chengdu (CN); Pingfan Ma, Chengdu (CN); Haijun Tian, CRANBURY, NJ (US); Yangde Chen, CRANBURY, NJ (US); Tongtong Xue, Chengdu (CN); Liang Xiao, Chengdu (CN); Junyou Ge, Chengdu (CN); Jingyi Wang, Chengdu (CN)

(73) Assignee: CHENGDU KELUN PRECISION BIOTECHNOLOGY CO., LTD., Chengdu, Sichuan (CN)

(21) Appl. No.: 18/245,539

(22) PCT Filed: Oct. 11, 2021
(86) PCT No.: PCT/CN2021/123047

§ 371 Date: Mar. 15, 2023

(30) Foreign Application Priority Data
Oct. 15, 2020 (CN) 202011105495.6

(51) Int. Cl. A61K 40/42; A61K 39/395; A61K 40/31; A61P 35/00; C07K 14/725; C07K 16/30

(52) U.S. Cl. CPC A61K 40/4255; A61K 39/3955; A61K 40/31; A61P 35/00; C07K 14/7051; C07K 16/30; C07K 2317/569; C07K 2317/622

(57) ABSTRACT

The present invention relates to the field of biomedicine, and in particular, the present invention relates to an antibody or antigen-binding fragment thereof which specifically binds to MSLN, and a chimeric antigen receptor (CAR) comprising said antibody or antigen-binding fragment thereof. The present invention also relates to modified immune cells expressing said CAR, or co-expressing said CAR and additional bioactive molecules (e.g. PD-1 antibody and/or mIL-15), and a method for preparing said modified immune cells. The present invention also relates to the use of such antibodies, CARs, and immune cells for the prevention and/or treatment of diseases associated with the expression of mesothelin, such as malignant pleural mesothelioma, pancreatic cancer, lung cancer, breast cancer, and ovarian cancer, and a method for the prevention and/or treatment of diseases associated with the expression of mesothelin, such as malignant pleural mesothelioma, pancreatic cancer, lung cancer, breast cancer, ovarian cancer, and other MSLN-positive tumors.

### FIELD OF THE INVENTION

The present invention relates to the field of biological medicine, in particular, the present invention relates to antibodies or antigen-binding fragments thereof that specifically bind to MSLN, and chimeric antigen receptors (CARs) comprising the antibodies or antigen-binding fragments thereof. The present invention also relates to engineered immune cells expressing the CARs. or co-expressing the CARs and additional biologically active molecules (e.g., PD-1 antibody and/or mIL-15), and a method for preparing the engineered immune cells. The present invention also relates to use of the antibodies, CARs and immune cells for the prevention and/or treatment of diseases associated with mesothelin expression, for example cancers such as malignant pleural mesothelioma, pancreatic cancer, lung cancer, breast cancer, ovarian cancer, as well as a method for the prevention and/or treatment of MSLN-positive tumors, such as malignant pleural mesothelioma, pancreatic cancer, lung cancer, breast cancer, and ovarian cancer.

### BACKGROUND OF THE INVENTION

Mesothelin (MSLN) is a glycosylphosphatidylinositol-linked glycoprotein. The precursor protein of MSLN is hydrolyzed by protease into 31 kDa megakaryocyte-potentiating factor (MPF) and 40 kDa mesothelin. Preliminary studies have shown that CA125/MUC16 is a ligand of mesothelin, which binds to mesothelin through the repeats of the N-terminal extracellular domain and participates in cell adhesion together. MSLN has highly specific expression. It has low expression on mesothelial cells of normal tissues such as peritoneal cavity, pleural cavity and pericardial cavity, while has high expression in solid tumors such as malignant pleural mesothelioma, pancreatic cancer, lung cancer, breast cancer, and ovarian cancer, especially higher expression in malignant mesothelioma (85%-90%), pancreatic cancer (80%-85%), epithelial ovarian cancer (60%-65%) and lung cancer (60%-65%), which is related to cell proliferation, cell adhesion function and anti-apoptotic process. These biological characteristics suggest that MSLN may serve as an ideal tumor therapy target for multiple indications.

With the increasing incidence of cancers year by year, the traditional therapeutic methods such as surgery, radiotherapy and chemotherapy show poor effectiveness in tumor treatment, and there is an urgent need for effective methods for tumor treatment. Chimeric antigen receptor (CAR)-T cell therapy is regarded as one of the most promising cancer treatments and has become a new hope for mankind to fight cancers. It achieves the purpose of treating tumors in a non-MHC-restricted manner by in vitro culture of immune cells collected from patients, in vitro transduction with a specific exogenous gene, in vitro expansion and then infusion back into the patients. The CAR-T cell therapy has achieved remarkable efficacy in the treatment of hematological malignancies, with a complete remission rate of over 90% for relapsed and refractory B-cell leukemia. Solid tumors account for about 90% of all malignant tumors, and their therapeutic drugs are in great demand. However, the current therapeutic effect of CAR-T cell therapy in solid tumors is still insufficient, mainly due to the complex tumor microenvironment and high tumor heterogeneity of solid tumors.

Based on the specificity of MSLN expression, MSLN-targeting CAR-T cell therapy is expected to become one of the ways to overcome MSLN-positive tumors. Therefore, it is urgent and necessary to develop a MSLN-targeting CAR-T therapy with high specificity and good efficacy.

### CONTENT OF THE INVENTION

In the present application, the inventors first developed a fully human antibody with low immunogenicity which is capable of specifically recognizing/binding to MSLN. On this basis, the present invention designs and constructs a chimeric antigen receptor (CAR) comprising the MSLN antibody or antigen-binding fragment thereof, and further designs and constructs a MSLN-targeting CAR co-expressed with PD-1 antibody and/or mIL-15. The CAR of the present invention is capable of directing the specificity and reactivity of an immune effector cell to MSLN-expressing cells (e.g., cells of malignant pleural mesothelioma, pancreatic cancer, lung cancer, breast cancer, ovarian cancer) in a non-MHC-restricted manner so as to cause the elimination of the MSLN-expressing cell. Therefore, the MSLN-targeting CAR of the present invention has the potential for preventing and/or treating a disease associated with mesothelin expression, such as malignant pleural mesothelioma, pancreatic cancer, lung cancer, breast cancer, ovarian cancer and other MSLN-positive tumor, with significant clinical value.

### Antibodies of the Present Invention

A first aspect of the present invention provides an antibody or antigen-binding fragment thereof capable of specifically binding to MSLN (e.g., human MSLN).

In certain embodiments, the antibody or antigen-binding fragment thereof of the present invention comprises:
(1a) the following three heavy chain CDRs defined by the Kabat numbering system: CDR-H1 having the sequence as set forth in SEQ ID NO: 3 or a variant thereof: CDR-H2 having the sequence as set forth in SEQ ID NO: 4 or a variant thereof: CDR-H3 having the sequence as set forth in SEQ ID NO: 5 or a variant thereof; and/or, the following three light chain CDRs defined by the Kabat numbering system: CDR-L1 having the sequence as set forth in SEQ ID NO: 6 or a variant thereof: CDR-L2 having the sequence as set forth in SEQ ID NO: 7 or a variant thereof; CDR-L3 having the sequence as set forth in SEQ ID NO: 8 or a variant thereof;
or
(1b) the following three heavy chain CDRs defined by the IMGT numbering system: CDR-H1 having the sequence as set forth in SEQ ID NO: 9 or a variant thereof; CDR-H2 having the sequence as set forth in SEQ ID NO: 10 or a variant thereof; CDR-H3 having the sequence as set forth in SEQ ID NO: 11 or a variant thereof; and/or, the following three light chain CDRs defined by the IMGT numbering system: CDR-L1 having the sequence as set forth in SEQ ID NO: 12 or a variant thereof; CDR-L2 having the sequence as set forth in SEQ ID NO: 13 or a variant thereof; CDR-L3 having the sequence as set forth in SEQ ID NO: 8 or a variant thereof;
or
(1c) the following three heavy chain CDRs defined by the Chothia numbering system: CDR-H1 having the sequence as set forth in SEQ ID NO: 14 or a variant thereof; CDR-H2 having the sequence as set forth in SEQ ID NO: 15 or a variant thereof; CDR-H3 having the sequence as set forth in SEQ ID NO: 5 or a variant thereof; and/or, the following three light chain CDRs defined by the Chothia numbering system: CDR-L1 having the sequence as set forth in SEQ ID NO: 6 or a variant thereof; CDR-L2 having the sequence as set forth in SEQ ID NO: 7 or a variant thereof; CDR-L3 having the sequence as set forth in SEQ ID NO: 8 or a variant thereof;
wherein the variant described in any of (1a), (1b), (1c) has a substitution, deletion or addition of one or several amino acids (e.g., a substitution, deletion or addition of 1, 2 or 3 amino acids) as compared to the sequence from which it is derived: preferably, the substitution is a conservative substitution.

In certain embodiments, the antibody or antigen-binding fragment thereof comprises:
(a) the following six heavy chain and light chain CDRs defined by the Kabat numbering system: CDR-H1 having the sequence as set forth in SEQ ID NO: 3: CDR-H2 having the sequence as set forth in SEQ ID NO: 4: CDR-H3 having the sequence as set forth in SEQ ID NO: 5: CDR-L1 having the sequence as set forth in SEQ ID NO: 6: CDR-L2 having the sequence as set forth in SEQ ID NO: 7: CDR-L3 having the sequence as set forth in SEQ ID NO: 8: or
(b) the following six heavy chain and light chain CDRs defined by the IMGT numbering system: CDR-H1 having the sequence as set forth in SEQ ID NO: 9: CDR-H2 having the sequence as set forth in SEQ ID NO: 10: CDR-H3 having the sequence as set forth in SEQ ID NO: 11: CDR-L1 having the sequence as set forth in SEQ ID NO: 12: CDR-L2 having the sequence as set forth in SEQ ID NO: 13: CDR-L3 having the sequence as set forth in SEQ ID NO: 8: or
(c) the following six heavy chain and light chain CDRs defined by the Chothia numbering system: CDR-H1 having the sequence as set forth in SEQ ID NO: 14: CDR-H2 having the sequence as set forth in SEQ ID NO: 15: CDR-H3 having the sequence as set forth in SEQ ID NO: 5: CDR-L1 having the sequence as set forth in SEQ ID NO: 6: CDR-L2 having the sequence as set forth in SEQ ID NO: 7: CDR-L3 having the sequence as set forth in SEQ ID NO: 8.

In certain embodiments, the antibody or antigen-binding fragment thereof comprises framework regions (FRs) derived from a human immunoglobulin.

In certain embodiments, the antibody or antigen-binding fragment thereof comprises:
a VH comprising the sequence as set forth in SEQ ID NO: 1 or a variant thereof and/or a VL comprising the sequence as set forth in SEQ ID NO: 2 or a variant thereof; wherein the variant has a sequence identity of at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or has a substitution, deletion or addition of one or several amino acids (e.g., a substitution, deletion or addition of 1, 2, 3, 4 or 5 amino acids) as compared to the sequence from which it is derived: preferably, the substitution is a conservative substitution.

In certain embodiments, the antibody or antigen-binding fragment thereof of the present invention comprises:
(2a) the following three heavy chain CDRs defined by the Kabat numbering system: CDR-H1 having the sequence as set forth in SEQ ID NO: 18 or a variant thereof; CDR-H2 having the sequence as set forth in SEQ ID NO: 19 or a variant thereof; CDR-H3 having the sequence as set forth in SEQ ID NO: 20 or a variant thereof; and/or, the following three light chain CDRs defined by the Kabat numbering system: CDR-L1 having the sequence as set forth in SEQ ID NO: 21 or a variant thereof; CDR-L2 having the sequence as set forth in SEQ ID NO: 22 or a variant thereof; CDR-L3 having the sequence as set forth in SEQ ID NO: 23 or a variant thereof;
or
(2b) the following three heavy chain CDRs defined by the IMGT numbering system: CDR-H1 having the sequence as set forth in SEQ ID NO: 24 or a variant thereof; CDR-H2 having the sequence as set forth in SEQ ID NO: 25 or a variant thereof; CDR-H3 having the sequence as set forth in SEQ ID NO: 26 or a variant thereof; and/or, the following three light chain CDRs defined by the IMGT numbering system: CDR-L1 having the sequence as set forth in SEQ ID NO: 27 or a variant thereof; CDR-L2 having the sequence as set forth in SEQ ID NO: 28 or a variant thereof; CDR-L3 having the sequence as set forth in SEQ ID NO: 23 or a variant thereof;
or
(2c) the following three heavy chain CDRs defined by the Chothia numbering system: CDR-H1 having the sequence as set forth in SEQ ID NO: 29 or a variant thereof; CDR-H2 having the sequence as set forth in SEQ ID NO: 30 or a variant thereof; CDR-H3 having the sequence as set forth in SEQ ID NO: 20 or a variant thereof; and/or, the following three light chain CDRs defined by the Chothia numbering system: CDR-L1 having the sequence as set forth in SEQ ID NO: 21 or a variant thereof; CDR-L2 having the sequence as set forth in SEQ ID NO: 22 or a variant thereof; CDR-L3 having the sequence as set forth in SEQ ID NO: 23 or a variant thereof;
wherein the variant described in any one of (2a), (2b), (2c) has a substitution, deletion or addition of one or several amino acids (e.g., a substitution, deletion or addition of 1, 2 or 3 amino acids) as compared to the sequence from which it is derived: preferably, the substitution is a conservative substitution.

In certain embodiments, the antibody or antigen-binding fragment thereof comprises:
(a) the following six heavy chain and light chain CDRs defined by the Kabat numbering system: CDR-H1 having the sequence as set forth in SEQ ID NO: 18: CDR-H2 having the sequence as set forth in SEQ ID NO: 19: CDR-H3 having the sequence as set forth in SEQ ID NO: 20: CDR-L1 having the sequence as set forth in SEQ ID NO: 21: CDR-L2 having the sequence as set forth in SEQ ID NO: 22: CDR-L3 having the sequence as set forth in SEQ ID NO: 23: or
(b) the following six heavy chain and light chain CDRs defined by the IMGT numbering system: CDR-H1 having the sequence as set forth in SEQ ID NO: 24: CDR-H2 having the sequence as set forth in SEQ ID NO: 25: CDR-H3 having the sequence as set forth in SEQ ID NO: 26: CDR-L1 having the sequence as set forth in SEQ ID NO: 27: CDR-L2 having the sequence as set forth in SEQ ID NO: 28: CDR-L3 having the sequence as set forth in SEQ ID NO: 23; or
(c) the following six heavy chain and light chain CDRs defined by the Chothia numbering system: CDR-H1 having the sequence as set forth in SEQ ID NO: 29: CDR-H2 having the sequence as set forth in SEQ ID NO: 30: CDR-H3 having the sequence as set forth in SEQ ID NO: 20: CDR-L1 having the sequence as set forth in SEQ ID NO: 21: CDR-L2 having the sequence as set forth in SEQ ID NO: 22: CDR-L3 having the sequence as set forth in SEQ ID NO: 23.

In certain embodiments, the antibody or antigen-binding fragment thereof comprises framework regions (FRs) derived from a human immunoglobulin.

In certain embodiments, the antibody or antigen-binding fragment thereof comprises:
a VH comprising the sequence as set forth in SEQ ID NO: 16 or a variant thereof and/or a VL comprising the sequence as set forth in SEQ ID NO: 17 or a variant thereof; wherein the variant has a sequence identity of at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or have a substitution, deletion or addition of one or several amino acids (e.g., a substitution, deletion or addition of 1, 2, 3, 4 or 5 amino acids) as compared to the sequence from which it is derived: preferably, the substitution is a conservative substitution.

In certain embodiments, the antibody or antigen-binding fragment thereof of the present invention comprises:
(3a) the following three heavy chain CDRs defined by the Kabat numbering system: CDR-H1 having the sequence as set forth in SEQ ID NO: 33 or a variant thereof; CDR-H2 having the sequence as set forth in SEQ ID NO: 34 or a variant thereof; CDR-H3 having the sequence as set forth in SEQ ID NO: 35 or a variant thereof; and/or, the following three light chain CDRs defined by the Kabat numbering system: CDR-L1 having the sequence as set forth in SEQ ID NO: 36 or a variant thereof; the CDR-L2 having the sequence as set forth in SEQ ID NO: 37 or a variant thereof; CDR-L3 having the sequence as set forth in SEQ ID NO: 38 or a variant thereof;
or
(3b) the following three heavy chains CDRs defined by the IMGT numbering system: CDR-H1 having the sequence as set forth in SEQ ID NO: 39 or a variant thereof; CDR-H2 having the sequence as set forth in SEQ ID NO: 40 or a variant thereof; CDR-H3 having the sequence as set forth in SEQ ID NO: 41 or a variant thereof; and/or, the following three light chain CDRs defined by the IMGT numbering system: CDR-L1 having the sequence as set forth in SEQ ID NO: 42 or a variant thereof; CDR-L2 having the sequence as set forth in SEQ ID NO: 43 or a variant thereof; CDR-L3 having the sequence as set forth in SEQ ID NO: 38 or a variant thereof;
or
(3c) the following three heavy chain CDRs defined by the Chothia numbering system: CDR-H1 having the sequence as set forth in SEQ ID NO: 44 or a variant thereof; CDR-H2 having the sequence as set forth in SEQ ID NO: 45 or a variant thereof; CDR-H3 having the sequence as set forth in SEQ ID NO: 35 or a variant thereof; and/or, the following three light chain CDRs defined by the Chothia numbering system: CDR-L1 having the sequence as set forth in SEQ ID NO: 36 or a variant thereof; CDR-L2 having the sequence as set forth in SEQ ID NO: 37 or a variant thereof; CDR-L3 having the sequence as set forth in SEQ ID NO: 38 or a variant thereof;
wherein the variant described in any one of (3a), (3b), (3c) has a substitution, deletion or addition of one or several amino acids (e.g., a substitution, deletion or addition of 1, 2 or 3 amino acids) as compared to the sequence from which it is derived: preferably, the substitution is a conservative substitution.

In certain embodiments, the antibody or antigen-binding fragment thereof comprises:
(a) the following six heavy chain and light chain CDRs defined by the Kabat numbering system: CDR-H1 having the sequence as set forth in SEQ ID NO: 33: CDR-H2 having the sequence as set forth in SEQ ID NO: 34: CDR-H3 having the sequence as set forth in SEQ ID NO: 35: CDR-L1 having the sequence as set forth in SEQ ID NO: 36: CDR-L2 having the sequence as set forth in SEQ ID NO: 37: CDR-L3 having the sequence as set forth in SEQ ID NO: 38; or
(b) the following six heavy chain and light chain CDRs defined by the IMGT numbering system: CDR-H1 having the sequence as set forth in SEQ ID NO: 39: CDR-H2 having the sequence as set forth in SEQ ID NO: 40: CDR-H3 having the sequence as set forth in SEQ ID NO: 41: CDR-L1 having the sequence as set forth in SEQ ID NO: 42: CDR-L2 having the sequence as set forth in SEQ ID NO: 43: CDR-L3 having the sequence as set forth in SEQ ID NO: 38; or
(c) the following six heavy chain and light chain CDRs defined by the Chothia numbering system: CDR-H1 having the sequence as set forth in SEQ ID NO: 44: CDR-H2 having the sequence as set forth in SEQ ID NO: 45: CDR-H3 having the sequence as set forth in SEQ ID NO: 35: CDR-L1 having the sequence as set forth in SEQ ID NO: 36: CDR-L2 having the sequence as set forth in SEQ ID NO: 37: CDR-L3 having the sequence as set forth in SEQ ID NO: 38.

In certain embodiments, the antibody or antigen-binding fragment thereof comprises framework regions (FRs) derived from a human immunoglobulin.

In certain embodiments, the antibody or antigen-binding fragment thereof comprises:
a VH comprising the sequence as set forth in SEQ ID NO: 31 or a variant thereof and/or a VL comprising the sequence as set forth in SEQ ID NO: 32 or a variant thereof; wherein the variant has a sequence identity of at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or has a substitution, deletion or addition of one or several amino acids (e.g., a substitution, deletion or addition of 1, 2, 3, 4 or 5 amino acids) as compared to the sequence from which it is derived: preferably, the substitution is a conservative substitution.

In certain embodiments, the antibody or antigen-binding fragment thereof of the present invention may be selected from camelid Ig, IgNAR, Fab fragment, Fab′ fragment, F(ab)′₂ fragment, F(ab)′₃ fragment, single chain antibody (e.g., scFv, di-scFv or (scFv)₂), minibody, bifunctional antibody, trifunctional antibody, tetrafunctional antibody, disulfide-stabilized Fv protein (dsFv) and single domain antibody (sdAb, nanobody).

In certain embodiments, the antibody of the present invention may be an antigen-binding fragment (e.g., scFv) that comprises one or more linkers, in which the one or more linkers connect two antibody domains or regions (e.g., a heavy chain variable (VH) region and a light chain variable (VL) region). Thus, the antibody of the present invention may comprise single chain antibody fragment, such as scFv and bifunctional antibody, particularly human single chain antibody fragment, which typically comprises one or more linkers that connect two antibody domains or regions (e.g., VH and VL regions).

The linker is typically a peptide linker, for example, a flexible and/or soluble peptide linker, for example, a peptide linker rich in glycine and serine. The linker comprises those linkers that are rich in glycine and serine and/or in some cases threonine. In some embodiments, the linker also comprises a charged residue (e.g., lysine and/or glutamic acid), which can improve solubility. In some embodiments, the linker further comprises one or more prolines.

In certain embodiments, the linker rich in glycine and serine (and/or threonine) comprises at least 80%, 85%, 90%, 91%, 92%, 93%, 94%, 95%, 96% %, 97%, 98% or 99% of such amino acids. In some embodiments, they comprise at least or about 50%, 55%, 60%, 70% or 75% of glycine, serine and/or threonine. In some embodiments, the linker consists essentially of glycine, serine and/or threonine. For example, the linker comprises a linker having single or repeats (e.g., 1, 2, 3, 4, and 5 repeats) of the sequence GGGGS or GGGS.

In certain embodiments, the VH and VL of the antibody or antigen-binding fragment thereof of the present invention are linked by one or more linkers: preferably, the linker comprises one or several (e.g., 1, 2 or 3) sequences as set forth in (GₘS)ₙ, wherein m is an integer selected from 1 to 6, and n is an integer selected from 1 to 6: preferably, m is 3, 4, or 5: preferably, n is 1 or 2; more preferably, the linker has the sequence as set forth in SEQ ID NO:52.

In certain embodiments, the antibody or antigen-binding fragment thereof is a single chain antibody, for example, scFv, di-scFv, or (scFv) 2.

In certain embodiments, the single chain antibody comprises, in order from its N-terminal to its C-terminal:
(1) a VH comprising the sequence as set forth in SEQ ID NO: 1 or a variant thereof—linker—a VL comprising the sequence as set forth in SEQ ID NO: 2 or a variant thereof;
(2) a VH comprising the sequence as set forth in SEQ ID NO: 16 or a variant thereof—linker—a VL comprising the sequence as set forth in SEQ ID NO: 17 or a variant thereof;
(3) a VH comprising the sequence as set forth in SEQ ID NO: 31 or a variant thereof—linker—a VL comprising the sequence as set forth in SEQ ID NO: 32 or a variant thereof;
(4) a VL comprising the sequence as set forth in SEQ ID NO: 2 or a variant thereof—linker—a VH comprising the sequence as set forth in SEQ ID NO: 1 or a variant thereof;
(5) a VL comprising the sequence as set forth in SEQ ID NO: 17 or a variant thereof—linker—a VH comprising the sequence as set forth in SEQ ID NO: 16 or a variant thereof; or
(6) a VL comprising the sequence as set forth in SEQ ID NO: 32 or a variant thereof—linker—a VH comprising the sequence as set forth in SEQ ID NO: 31 or a variant thereof;
wherein the variant has a sequence identity of at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or has a substitution, deletion or addition of one or several amino acids (e.g., a substitution, deletion or addition of 1, 2, 3, 4 or 5 amino acids) as compared to the sequence from which it is derived: preferably, the substitution is a conservative substitution.

In certain embodiments, the single chain antibody comprises an amino acid sequence selected from the group consisting of: (1) an amino acid sequence as set forth in any one of SEQ ID NOS: 54, 56, 58: (2) an amino acid sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence as set forth in any one of SEQ ID NOs: 54, 56, 58: or (3) a sequence having a substitute, deletion, or addition of one or several amino acids (e.g., a substitution, deletion or addition of 1, 2, 3, 4, 5, 6, 7, 8, 9, or 10 amino acids) as compared to the amino acid sequence as set forth in any one of SEQ ID NOs: 54, 56, 58: preferably, the substitution is a conservative substitution.

In certain embodiments, the antibody or antigen-binding fragment thereof of the present invention further comprises a constant region derived from a human immunoglobulin. In certain embodiments, the heavy chain of the antibody or antigen-binding fragment thereof comprises a heavy chain constant region derived from a human immunoglobulin (e.g., IgG1, IgG2, IgG3 or IgG4), and the light chain of the antibody or antigen-binding fragment thereof comprises a light chain constant region derived from a human immunoglobulin (e.g., κ or λ).

In certain embodiments, the heavy chain of the antibody or antigen-binding fragment thereof comprises a heavy chain constant region (CH) of human immunoglobulin or a variant thereof, the variant has a substitution, deletion, or addition of one or more amino acids (e.g., a substitution, deletion, or addition of up to 20, up to 15, up to 10, or up to 5 amino acids: e.g., a substitution, deletion, or addition of 1, 2, 3, 4 or 5 amino acids) as compared to the wild-type sequence from which it is derived; and/or,
the light chain of the antibody or antigen-binding fragment thereof comprises a light chain constant region (CL) of a human immunoglobulin or a variant thereof, the variant has a substitution, deletion, or addition of one or more amino acids (e.g., a substitution, deletion, or addition of up to 20, up to 15, up to 10, or up to 5 amino acids: e.g., a substitution, deletion, or addition of 1, 2, 3, 4 or 5 amino acids) as compared to the wild-type sequence from which it is derived.

In certain embodiments, the heavy chain constant region is an IgG, IgM, IgE, IgD or IgA heavy chain constant region. In certain embodiments, the heavy chain constant region is an IgG heavy chain constant region, for example, an IgG1, IgG2, IgG3 or IgG4 heavy chain constant region.

In certain embodiments, the light chain constant region is a κ or λ light chain constant region. In certain preferred embodiments, the light chain constant region is a human κ light chain constant region.

### Preparation of Antibody

The antibody of the present invention can be prepared by various methods known in the art, such as by genetic engineering recombinant techniques. For example, a DNA molecule encoding the heavy and light chain genes of the antibody of the present invention can be obtained by chemical synthesis or PCR amplification: the resulting DNA molecule can be inserted into an expression vector, and then transfected into a host cell. Then, the transfected host cell can be cultured under a specific condition to express the antibody of the present invention.

The antigen-binding fragment of the present invention can be obtained by hydrolysis of an intact antibody molecule (see: Morimoto et al., J. Biochem. Biophys. Methods 24:107-117 (1992) and Brennan et al., Science 229:81 (1985)). Alternatively, the antigen-binding fragment can also be produced directly from a recombinant host cell (reviewed in Hudson, Curr. Opin. Immunol. 11:548-557 (1999): Little et al., Immunol. Today, 21:364-370 (2000)). For example, a Fab′ fragment can be obtained directly from a host cell: a F(ab′)₂ fragment can be formed by chemically coupling Fab′ fragments (Carter et al., Bio/Technology, 10:163-167 (1992)). In addition, a Fv, Fab or F(ab′)₂ fragment can also be directly isolated from a culture medium of recombinant host cells. Other techniques for preparing these antigen-binding fragments are well known to those of ordinary skill in the art.

Accordingly, a second aspect of the present invention provides an isolated nucleic acid molecule, which comprises a nucleotide sequence encoding the antibody or antigen-binding fragment thereof of the present invention or a heavy chain variable region and/or a light chain variable region thereof.

In certain embodiments, the isolated nucleic acid molecule comprises a nucleotide sequence selected from the group consisting of: (1) a nucleotide sequence as set forth in any one of SEQ ID NOs: 55, 57 and 59: (2) a nucleotide sequence having a sequence identity of at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the nucleotide sequence as set forth in any one of SEQ ID NOs: 55, 57 and 59.

A third aspect of the present invention provides a vector (e.g., a cloning vector or an expression vector), which comprises the isolated nucleic acid molecule as described above. In certain embodiments, the vector of the present invention is, for example, a DNA vector, an RNA vector, a plasmid, a transposon vector, a CRISPR/Cas9 vector or a viral vector: preferably, the vector is an expression vector: preferably, the vector is an episomal vector: preferably, the vector is a viral vector: more preferably, the viral vector is a lentiviral, adenoviral or retroviral vector.

A fourth aspect of the present invention provides a host cell, which comprises the isolated nucleic acid molecule or vector as described above. Such host cell includes, but is not limited to, prokaryotic cell such as E. coli cell, and eukaryotic cell such as yeast cell, insect cell, plant cell, and animal cell (e.g., mammalian cell, for example, mouse cell, human cell, etc.).

In another aspect, the present invention also relates to a method for preparing the antibody or antigen-binding fragment thereof of the present invention, which comprises culturing the host cell as described above under conditions permitting the expression of the antibody or antigen-binding fragment thereof, and recovering the antibody or antigen-binding fragment thereof from a culture of the cultured host cell.

### Chimeric Antigen Receptor (CAR)

The present invention relates to a MSLN-targeting CAR, which is featured with an ability of recognizing MSLN in a non-MHC-restricted manner, which confers on an immune cell (e.g., T cell, NK cell, monocyte, macrophage, or dendritic cell) that expresses the CAR an ability to recognize an MSLN-expressing cell (e.g., tumor cell) without relying on antigen processing and presentation.

Therefore, a fifth aspect of the present invention provides a chimeric antigen receptor (CAR), which comprises an extracellular antigen-binding domain (anti-MSLN binding domain), a spacer domain, a transmembrane domain and an intracellular signaling domain.

### I. Extracellular Antigen-Binding Domain

The antigen-binding domain contained in the chimeric antigen receptor of the present invention confers on the CAR an ability to recognize MSLN.

In certain embodiments, the antigen-binding domain comprises an anti-MSLN binding domain, and the anti-MSLN binding domain comprises an antibody or antigen-binding fragment thereof capable of specifically binding to MSLN (e.g., human MSLN). In certain embodiments, the antibody or antigen-binding fragment thereof is selected from the antibody or antigen-binding fragment thereof of the first aspect.

In certain embodiments, the anti-MSLN binding domain comprises a VH and a VL, wherein,
the VH comprises the following three heavy chain CDRs defined by the Kabat numbering system: CDR-H1 having the sequence as set forth in SEQ ID NO: 3 or a variant thereof; CDR-H2 having the sequence as set forth in SEQ ID NO: 4 or a variant thereof; CDR-H3 having the sequence as set forth in SEQ ID NO: 5 or a variant thereof; the VL comprises the following three light chain CDRs defined by the Kabat numbering system: CDR-L1 having the sequence as set forth in SEQ ID NO: 6 or a variant thereof; CDR-L2 having the sequence as set forth in SEQ ID NO: 7 or a variant thereof; CDR-L3 having the sequence as set forth in SEQ ID NO: 8 or a variant thereof; or,
the VH comprises the following three heavy chain CDRs defined by the IMGT numbering system: CDR-H1 having the sequence as set forth in SEQ ID NO: 9 or a variant thereof; CDR-H2 having the sequence as set forth in SEQ ID NO: 10 or a variant thereof; CDR-H3 having the sequence as set forth in SEQ ID NO: 11 or a variant thereof; the VL comprises the following three light chain CDRs defined by the IMGT numbering system: CDR-L1 having the sequence as set forth in SEQ ID NO: 12 or a variant thereof; CDR-L2 having the sequence as set forth in SEQ ID NO: 13 or a variant thereof; CDR-L3 having the sequence as set forth in SEQ ID NO: 8 or a variant thereof; or,
the VH comprises the following three heavy chain CDRs defined by the Chothia numbering system: CDR-H1 having the sequence as set forth in SEQ ID NO: 14 or a variant thereof; CDR-H2 having the sequence as set forth in SEQ ID NO: 15 or a variant thereof; CDR-H3 having the sequence as set forth in SEQ ID NO: 5 or a variant thereof; the VL comprises the following three light chain CDRs defined by the Chothia numbering system: CDR-L1 having the sequence as set forth in SEQ ID NO: 6 or a variant thereof; CDR-L2 having the sequence as set forth in SEQ ID NO: 7 or a variant thereof; CDR-L3 having the sequence as set forth in SEQ ID NO: 8 or a variant thereof;
wherein the variant has a substitution, deletion or addition of one or several amino acids (e.g., a substitution, deletion or addition of 1, 2 or 3 amino acids) compared to the sequence from which it is derived: preferably, the substitution is a conservative substitution.

In certain embodiments, the VH comprises the sequence as set forth in SEQ ID NO: 1 or a variant thereof, and the VL comprises the sequence as set forth in SEQ ID NO: 2 or a variant thereof; wherein the variant has a sequence identity of at least 80%, at least 85%, at least 90%, at least 91%, at least 92%, at least 93%, at least 94%, at least 95%, at least 96%, at least 97%, at least 98%, at least 99%, or 100% as compared to the sequence from which it was derived, or has a substitution, deletion or addition of one or several amino acids (e.g., a substitution, deletion or addition of 1, 2, 3, 4 or 5 amino acids) as compared to the sequence from which it is derived; preferably, the substitution is a conservative substitution.

In certain embodiments, the anti-MSLN binding domain comprises a VH and a VL, wherein,
the VH comprises the following three heavy chain CDRs defined by the Kabat numbering system: CDR-H1 having the sequence as set forth in SEQ ID NO: 18 or a variant thereof; CDR-H2 having the sequence as set forth in SEQ ID NO: 19 or a variant thereof; CDR-H3 having the sequence as set forth in SEQ ID NO: 20 or a variant thereof; the VL comprises the following three light chain CDRs defined by the Kabat numbering system: CDR-L1 of the sequence as set forth in SEQ ID NO: 21 or a variant thereof; CDR-L2 having the sequence as set forth in SEQ ID NO: 22 or a variant thereof; CDR-L3 having the sequence as set forth in SEQ ID NO: 23 or a variant thereof; or,
the VH comprises the following three heavy chain CDRs defined by the IMGT numbering system: CDR-H1 having the sequence as set forth in SEQ ID NO: 24 or a variant thereof; CDR-H2 having the sequence as set forth in SEQ ID NO: 25 or a variant thereof; CDR-H3 having the sequence as set forth in SEQ ID NO: 26 or a variant thereof; the VL comprises the following three light chain CDRs defined by the IMGT numbering system: CDR-L1 having the sequence as set forth in SEQ ID NO: 27 or a variant thereof; CDR-L2 having the sequence as set forth in SEQ ID NO: 28 or a variant thereof; CDR-L3 having the sequence as set forth in SEQ ID NO: 23 or a variant thereof; or,
the VH comprises the following three heavy chain CDRs defined by the Chothia numbering system: CDR-H1 having the sequence as set forth in SEQ ID NO: 29 or a variant thereof; CDR-H2 having the sequence as set forth in SEQ ID NO: 30 or a variant thereof; CDR-H3 having the sequence as set forth in SEQ ID NO: 20 or a variant thereof; the VL comprises the following three light chain CDRs defined by the Chothia numbering system: CDR-L1 having the sequence as set forth in SEQ ID NO: 21 or a variant thereof; CDR-L2 having the sequence as set forth in SEQ ID NO: 22 or a variant thereof; CDR-L3 having the sequence as set forth in SEQ ID NO: 23 or a variant thereof;
wherein the variant has a substitution, deletion or addition of one or several amino acids (e.g., a substitution, deletion or addition of 1, 2 or 3 amino acids) compared to the sequence from which it is derived: preferably, the substitution is a conservative substitution.

In certain embodiments, the VH comprises the sequence as set forth in SEQ ID NO: 16 or a variant thereof, and the VL comprises the sequence as set forth in SEQ ID NO: 17 or a variant thereof; wherein the variant has a sequence identity of at least 80%, at least 85%, at least 90%, at least 91%, at least 92%, at least 93%, at least 94%, at least 95%, at least 96%, at least 97%, at least 98%, at least 99%, or 100% as compared to the sequence from which it was derived, or has a substitution, deletion or addition of one or several amino acids (e.g., a substitution, deletion or addition of 1, 2, 3, 4 or 5 amino acids) as compared to the sequence from which it is derived; preferably, the substitution is a conservative substitution.

In certain embodiments, the anti-MSLN binding domain comprises a VH and a VL, wherein,
the VH comprises the following three heavy chain CDRs defined by the Kabat numbering system: CDR-H1 having the sequence as set forth in SEQ ID NO: 33 or a variant thereof; CDR-H2 having the sequence as set forth in SEQ ID NO: 34 or a variant thereof; CDR-H3 having the sequence as set forth in SEQ ID NO: 35 or a variant thereof; the VL comprises the following three light chain CDRs defined by the Kabat numbering system: CDR-L1 having the sequence as set forth in SEQ ID NO: 36 or a variant thereof; CDR-L2 having the sequence as set forth in SEQ ID NO: 37 or a variant thereof; CDR-L3 having the sequence as set forth in SEQ ID NO: 38 or a variant thereof; or,
the VH comprises the following three heavy chain CDRs defined by the IMGT numbering system: CDR-H1 having the sequence as set forth in SEQ ID NO: 39 or a variant thereof; CDR-H2 having the sequence as set forth in SEQ ID NO: 40 or a variant thereof; CDR-H3 having the sequence as set forth in SEQ ID NO: 41 or a variant thereof; the VL comprises the following three light chain CDRs defined by the IMGT numbering system: CDR-L1 having the sequence as set forth in SEQ ID NO: 42 or a variant thereof; CDR-L2 having the sequence as set forth in SEQ ID NO: 43 or a variant thereof; CDR-L3 having the sequence as set forth in SEQ ID NO: 38 or a variant thereof; or,
the VH comprises the following three heavy chain CDRs defined by the Chothia numbering system: CDR-H1 having the sequence as set forth in SEQ ID NO: 44 or a variant thereof; CDR-H2 having the sequence as set forth in SEQ ID NO: 45 or a variant thereof; CDR-H3 having the sequence as set forth in SEQ ID NO: 35 or a variant thereof; the VL comprises the following three light chain CDRs defined by the Chothia numbering system: CDR-L1 having the sequence as set forth in SEQ ID NO: 36 or a variant thereof; CDR-L2 having the sequence as set forth in SEQ ID NO: 37 or a variant thereof; CDR-L3 having the sequence as set forth in SEQ ID NO: 38 or a variant thereof;
wherein the variant has a substitution, deletion or addition of one or several amino acids (e.g., a substitution, deletion or addition of 1, 2 or 3 amino acids) compared to the sequence from which it is derived: preferably, the substitution is a conservative substitution.

In certain embodiments, the VH comprises the sequence as set forth in SEQ ID NO: 31 or a variant thereof, and the VL comprises the sequence as set forth in SEQ ID NO: 32 or a variant thereof; wherein the variant has a sequence identity of at least 80%, at least 85%, at least 90%, at least 91%, at least 92%, at least 93%, at least 94%, at least 95%, at least 96%, at least 97%, at least 98%, at least 99%, or 100% as compared to the sequence from which it was derived, or has a substitution, deletion or addition of one or several amino acids (e.g., a substitution, deletion or addition of 1, 2, 3, 4 or 5 amino acids) as compared to the sequence from which it is derived; preferably, the substitution is a conservative substitution.

In certain embodiments, the anti-MSLN binding domain according to any one of the above embodiments is a single chain antibody, for example, a scFv, di-scFv, or (scFv) 2.

In certain embodiments, the VH and VL contained in the anti-MSLN binding domain described in any one of the above embodiments, or the VH and VL of the antibody or antigen-binding fragment thereof contained in the anti-MSLN binding domain are connected through a linker. In certain embodiments, the linker comprises one or several (e.g., 1, 2 or 3) sequences as set forth in (GₘS)ₙ, wherein m is an integer selected from 1 to 6, and n is an integer selected from 1 to 6. In certain embodiments, m is 3, 4 or 5. In certain embodiments, n is 1 or 2. In certain embodiments, the linker has the sequence as set forth in SEQ ID NO:52.

In certain embodiments, the anti-MSLN binding domain comprises a single chain antibody, and the single chain antibody comprises an amino acid sequence selected from the group consisting of:
(1) an amino acid sequence as set forth in SEQ ID NO: 54 or a variant thereof,
(2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence as set forth in SEQ ID NO: 54; or,
(3) a sequence having a substitution, deletion or addition of one or several amino acids (e.g., a substitution, deletion or addition of 1, 2, 3, 4, 5, 6, 7, 8, 9, or 10 amino acids) as compared to the amino acid sequence as set forth in SEQ ID NO: 54: preferably, the substitution is a conservative substitution.

In certain embodiments, the anti-MSLN binding domain comprises a single chain antibody, and the single chain antibody comprises an amino acid sequence selected from the group consisting of:
(1) an amino acid sequence as set forth in SEQ ID NO: 56 or a variant thereof,
(2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence as set forth in SEQ ID NO: 56; or,
(3) a sequence having a substitution, deletion or addition of one or several amino acids (e.g., a substitution, deletion or addition of 1, 2, 3, 4, 5, 6, 7, 8, 9, or 10 amino acids) as compared to the amino acid sequence as set forth in SEQ ID NO: 56: preferably, the substitution is a conservative substitution.

In certain embodiments, the anti-MSLN binding domain comprises a single chain antibody, and the single chain antibody comprises an amino acid sequence selected from the group consisting of:
(1) an amino acid sequence as set forth in SEQ ID NO: 58 or a variant thereof,
(2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence as set forth in SEQ ID NO: 58; or,
(3) a sequence having a substitution, deletion or addition of one or several amino acids (e.g., a substitution, deletion or addition of 1, 2, 3, 4, 5, 6, 7, 8, 9, or 10 amino acids) as compared to the amino acid sequence as set forth in SEQ ID NO: 58: preferably, the substitution is a conservative substitution.

In certain embodiments, the antigen-binding domain comprises the anti-MSLN binding domain as a first binding domain, and further comprises a second binding domain that does not bind to MSLN. In certain preferred embodiments, the antigen bound by the second binding domain that does not bind to MSLN is selected from the group consisting of: CD19, GPC3, PSMA, MUC1, EGFR, HER2, CD276, GD2, BCMA, CD33 or Claudin18.2.

### II. Transmembrane Domain

The transmembrane domain contained in the chimeric antigen receptor of the present invention can be any protein structure known in the art as long as it can be thermodynamically stable in cell membranes (especially eukaryotic cell membranes). Transmembrane domains suitable for use in the CAR of the present invention may be derived from a natural source. In such embodiments, the transmembrane domain can be derived from any membrane-bound or transmembrane protein. Alternatively, the transmembrane domain may be a synthetic non-naturally occurring protein segment, for example, a protein segment mainly comprising hydrophobic residues such as leucine and valine.

In certain embodiments, the transmembrane domain is a transmembrane region of a protein selected from the group consisting of: a, B or (chain of T cell receptor, CD28, CD45, CD3ε, CD3ζ, CD4, CD5, CD8α, CD9, CD16, CD22, CD33, CD37, CD64, CD80, CD86, CD134, CD137, CD152, CD154 and PD-1, as well as any combination thereof. In certain preferred embodiments, the transmembrane domain is a transmembrane region of a protein selected from the group consisting of: CD8a, CD4, PD1, CD152 and CD154. In certain preferred embodiments, the transmembrane domain comprises the transmembrane region of CD8α. In certain exemplary embodiments, the transmembrane domain comprises the amino acid sequence as set forth in SEQ ID NO: 64.

### III. Spacer Domain

The chimeric antigen receptor of the present invention comprises a spacer domain located between the extracellular antigen-binding domain and the transmembrane domain.

In certain embodiments, the spacer domain comprises the CH2 and CH3 regions of an immunoglobulin (e.g., IgG1 or IgG4). In such embodiments, without being bound by a particular theory, it is believed that CH2 and CH3 extend the antigen-binding domain of the CAR from the cell membrane of the cell expressing the CAR, and thus the size and domain structure of a native TCR may be more precisely mimicked.

In certain embodiments, the spacer domain comprises a hinge domain. The hinge domain can be an amino acid fragment commonly found between two domains in a protein, which may allow the protein to have flexibility and allow one or two domains to move relative to each other. Thus, the hinge domain can be any amino acid sequence as long as it can provide the extracellular antigen-binding domain with such flexibility and such mobility relative to the transmembrane domain.

In certain embodiments, the hinge domain is a hinge region of a naturally occurring protein, or a portion thereof. In certain embodiments, the hinge domain comprises the hinge region of CD8a, or a portion thereof, for example, a fragment comprising at least 15 (e.g., 20, 25, 30, 35, or 40) contiguous amino acids of the hinge region of CD8a. In certain exemplary embodiments, the spacer domain comprises the amino acid sequence as set forth in SEQ ID NO:62.

### IV. Signal Peptide

In certain embodiments, the CAR of the present invention may further comprise a signal peptide at its N-terminal. Typically, a signal peptide is a polypeptide sequence that targets the sequence to which it is linked to a desired site. In certain embodiments, the signal peptide can target the CAR to which it is linked to the secretory pathway of the cell, and allow the CAR to be further integrated and anchored into the lipid bilayer. Signal peptides useful in CARs are known to those of skill in the art. In certain embodiments, the signal peptide comprises a heavy chain signal peptide (e.g., heavy chain signal peptide of IgG1), a granulocyte-macrophage colony stimulating factor receptor 2 (GM-CSFR2) signal peptide, IL2 signal peptide, or a CD8a signal peptide. In certain preferred embodiments, the signal peptide is selected from CD8a signal peptides. In certain exemplary embodiments, the signal peptide comprises the amino acid sequence as set forth in SEQ ID NO:60.

In certain embodiments, the CAR of the present invention is co-expressed with an additional biologically active molecule. The additional biologically active molecule may have its own signal peptide, and this signal peptide is named as signal peptide-2 in order to distinguish it from the signal peptide described in the previous paragraph. The signal peptide-2 directs the transport of the additional biologically active molecule to a specific site within the cell or outside the cell membrane. The signal peptide-2 may be the same as or different from the signal peptide described in the previous paragraph. Preferably, the signal peptide-2 may be different from the signal peptide described in the previous paragraph.

### V. Intracellular Signaling Domain

The intracellular signaling domain contained in the CAR of the present invention is involved in transducing the signal generated by the combination of the CAR of the present invention with MSLN into an immune effector cell, activating at least one normal effector function of the CAR-expressing immune effector cell, or enhancing the secretion of at least one cytokine (e.g., IL-2, IFN-γ) by the CAR-expressing immune effector cell.

In certain embodiments, the intracellular signaling domain comprises a primary signaling domain and/or a costimulatory signaling domain.

In certain embodiments, the primary signaling domain may be any intracellular signaling domain comprising an immunoreceptor tyrosine activation motif (ITAM). In certain embodiments, the primary signaling domain comprises an immunoreceptor tyrosine activation motif (ITAM). In certain embodiments, the primary signaling domain comprises an intracellular signaling domain of a protein selected from the group consisting of: CD3ζ, FcRγ, FcRβ, CD3γ, CD3δ, CD3ε, CDS, CD22, CD79a, CD79b, or CD66d. In certain embodiments, the primary signaling domain comprises the intracellular signaling domain of CD3ζ.

In certain embodiments, the costimulatory signaling domain may be an intracellular signaling domain derived from a costimulatory molecule. In certain embodiments, the costimulatory signaling domain comprises an intracellular signaling domain of a protein selected from the group consisting of: CARD11, CD2, CD7, CD27, CD28, CD30, CD134 (OX40), CD137 (4-1BB), CD150 (SLAMF1), CD270 (HVEM), CD278 (ICOS) or DAP10.

In certain embodiments, the costimulatory signaling domain is selected from the intracellular signaling domain of CD28, or the intracellular signaling domain of CD137 (4-1BB), or a combination of fragments of both.

In certain embodiments, the intracellular signaling domain comprises one costimulatory signaling domain. In certain embodiments, the intracellular signaling domain comprises two or more costimulatory signaling domains. In such embodiments, the two or more costimulatory signaling domains may be the same or different.

In certain embodiments, the intracellular signaling domain comprises a primary signaling domain and at least one costimulatory signaling domain. The primary signaling domain and at least one costimulatory signaling domain can be tandemly linked in any order to the carboxy-terminal of the transmembrane domain.

In certain embodiments, the intracellular signaling domain may comprise the intracellular signaling domain of CD3ζ and the intracellular signaling domain of CD137. In certain exemplary embodiments, the intracellular signaling domain of CD3 ζ comprises the amino acid sequence as set forth in SEQ ID NO:68. In certain exemplary embodiments, the intracellular signaling domain of CD137 comprises the amino acid sequence as set forth in SEQ ID NO:66.

In certain exemplary embodiments, the intracellular signaling domain of the chimeric antigen receptor has the sequence as set forth in SEQ ID NO:70.

### VI. Full-Length CAR

The present invention provides a chimeric antigen receptor capable of specifically binding to MSLN, the chimeric antigen receptor comprises an anti-MSLN binding domain, a spacer domain, a transmembrane domain, an intracellular signaling domain from its N-terminal to its C-terminal in order. In certain preferred embodiments, the intracellular signaling domain therein comprise a costimulatory signaling domain and a primary signaling domain from the N-terminal to the C-terminal.

In certain embodiments, the spacer domain comprises the hinge region of CD8 (e.g., CD8α), which has the sequence as set forth in SEQ ID NO:62. In certain embodiments, the transmembrane domain comprises the transmembrane region of CD8 (e.g., CD8α), which has the sequence as set forth in SEQ ID NO:64.

In certain embodiments, the intracellular signaling domain comprises a primary signaling domain and a costimulatory signaling domain, wherein the primary signaling domain comprises the intracellular signaling domain of CD3ζ, which has the sequence as set forth in SEQ ID NO: 68. The costimulatory signaling domain comprises the intracellular signaling domain of CD137, which has the sequence as set forth in SEQ ID NO:66. In certain preferred embodiments, the intracellular signaling domain of the chimeric antigen receptor has the sequence as set forth in SEQ ID NO:70.

In certain preferred embodiments, the chimeric antigen receptor comprises, from its N-terminal to its C-terminal, the signal peptide, anti-MSLN binding domain, spacer domain, transmembrane domain, intracellular signaling domain (comprising a costimulatory signaling domain and a primary signaling domain from the N-terminal to the C-terminal) in order.

In certain exemplary embodiments, the signal peptide comprises the heavy chain signal peptide of IgG1 or the signal peptide of CD8α. In certain exemplary embodiments, the signal peptide comprises the signal peptide of CD8α having the sequence as set forth in SEQ ID NO:60.

In certain exemplary embodiments, the CAR of the present invention comprises an amino acid sequence selected from the group consisting of:
(1) an amino acid sequence as set forth in SEQ ID NO: 83,
(2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence as set forth in SEQ ID NO: 83, in which the sequence substantially retains at least one biological activity (e.g., ability to direct the specificity and reactivity of an immune effector cell to a MSLN-expressing cell in a non-MHC-restricted manner) of the amino acid sequence from which it is derived: or,
(3) a sequence having a substitution, deletion or addition of one or several amino acids (e.g., a substitution, deletion or addition of 1, 2, 3, 4, 5, 6, 7, 8, 9 or 10 amino acids) as compared to the amino acid sequence as set forth in SEQ ID NO: 83: preferably, the substitution is a conservative substitution.

In certain exemplary embodiments, the CAR of the present invention comprises an amino acid sequence selected from the group consisting of:
(1) an amino acid sequence as set forth in SEQ ID NO: 88,
(2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence as set forth in SEQ ID NO: 88, in which the sequence substantially retains at least one biological activity (e.g., ability to direct the specificity and reactivity of an immune effector cell to a MSLN-expressing cell in a non-MHC-restricted manner) of the amino acid sequence from which it is derived: or,
(3) a sequence having a substitution, deletion or addition of one or several amino acids (e.g., a substitution, deletion or addition of 1, 2, 3, 4, 5, 6, 7, 8, 9 or 10 amino acids) as compared to the amino acid sequence as set forth in SEQ ID NO: 88: preferably, the substitution is a conservative substitution.

In certain exemplary embodiments, the CAR of the present invention comprises an amino acid sequence selected from the group consisting of:
(1) an amino acid sequence as set forth in SEQ ID NO: 90,
(2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence as set forth in SEQ ID NO: 90, in which the sequence substantially retains at least one biological activity (e.g., ability to direct the specificity and reactivity of an immune effector cell to a MSLN-expressing cell in a non-MHC-restricted manner) of the amino acid sequence from which it is derived; or,
(3) a sequence having a substitution, deletion or addition of one or several amino acids (e.g., a substitution, deletion or addition of 1, 2, 3, 4, 5, 6, 7, 8, 9 or 10 amino acids) as compared to the amino acid sequence as set forth in SEQ ID NO: 90: preferably, the substitution is a conservative substitution.

### VII. Co-Expressed CAR and Additional Biologically Active Molecule

In some cases, the CAR of the fifth aspect of the present invention may also be co-expressed with an additional biologically active molecule. A self-cleaving peptide is able to prevent the formation of a covalent bond between amino acids during translation and keep translation going, so that the translation product is “self-cleaved”, and the chimeric antigen receptor specifically binding to MSLN is separated from the additional biologically active molecular. Therefore, when the CAR described in the fifth aspect of the present invention is co-expressed with an additional biologically active molecule, the chimeric antigen receptor that can specifically bind to MSLN becomes an independent CAR with an extracellular antigen-binding domain, a spacer domain, a transmembrane domain and an intracellular signaling domain, while the additional biologically active molecule can be secreted extracellularly or expressed as a membrane chimeric polypeptide or protein. With the expansion and enrichment of immune cells expressing CAR that specifically binds to MSLN in the tumor microenvironment, the additional biologically active molecule is also enriched in the tumor microenvironment and exerts synergistic anti-tumor effect with the anti-MSLN-CAR.

In certain embodiments, the additional biologically active molecule is one or more selected from the following components: an antibody or antigen-binding fragment thereof that specifically binds to an immune checkpoint (e.g., anti-PD-1, PD-L1, CTLA-4, or LAG-3 antibody or antigen-binding fragment thereof), cytokine (e.g., IL-15, IL-7, IL-12, IL-18, IL-21), or membrane-bound polypeptide (e.g., mIL-15, mIL-7, mIL-12, mIL-18, mIL-21).

In certain embodiments, the nucleic acid sequence encoding the anti-MSLN-CAR is linked to the nucleic acid sequence of the additional biologically active molecule via the nucleic acid sequence of the self-cleaving peptide. The anti-MSLN-CAR can be at the N-terminal or C-terminal of the additional biologically active molecule. In certain exemplary embodiments, the anti-MSLN-CAR is at the 5′-terminal of the additional biologically active molecule. Any self-cleaving peptide capable of causing cleavage of a fusion protein into two separate proteins can be used in the present invention. In certain exemplary embodiments, the self-cleaving peptide is P2A, preferably having the sequence as set forth in SEQ ID NO: 72, and its nucleotide sequence can be optimized according to the needs of gene recombination. In such embodiments, the fusion protein comprising the CAR and the additional biologically active molecule has the following structure:
N′-signal peptide-extracellular antigen-binding domain that specifically binds to human MSLN-spacer domain-transmembrane domain-intracellular signaling domain-self-cleaving peptide-signal peptide-2-additional biologically active molecule-C′, wherein the signal peptide-2 and the signal peptide at N′ are same or different.

In certain embodiments, the additional biologically active molecule has a signal peptide immediately at its upstream that is different from that at N-terminal of the MSLN-CAR. In certain exemplary embodiments, the signal peptide-2 is a human IL2 signal peptide having the sequence as set forth in SEQ ID NO: 74.

In certain embodiments, when the CAR of the present invention is co-expressed with more than one of the additional biologically active molecules, the plurality of nucleic acid sequences encoding the more than one additional biologically active molecules are linked with each other via a sequence encoding self-cleaving peptide (e.g., P2A).

In certain embodiments, the additional biologically active molecule is an anti-PD-1 linear antibody, preferably, the linear antibody has the sequence as set forth in SEQ ID NO:77.

In certain embodiments, the additional biologically active molecule is membrane-bound IL-15, preferably, the membrane-bound IL-15 has the sequence as set forth in SEQ ID NO:81.

### Preparation of Chimeric Antigen Receptor

Methods for generating a chimeric antigen receptor and an immune effector cell (e.g., T cell) comprising the chimeric antigen receptor are known in the art, and may comprise transfecting a cell with at least one polynucleotide encoding a CAR, and expressing the polynucleotide in the cell. For example, a nucleic acid molecule encoding the CAR of the present invention can be contained in an expression vector (e.g., a lentiviral vector), the expression vector can be expressed in a host cell, such as a T cell, to prepare the CAR.

Accordingly, a sixth aspect of the present invention provides an isolated nucleic acid molecule, which comprises a nucleotide sequence encoding the chimeric antigen receptor of the fifth aspect.

Those skilled in the art understand that, due to the degeneracy of genetic code, a nucleotide sequence encoding the chimeric antigen receptor of the present invention can have a variety of different sequences. Thus, unless stated otherwise, a “nucleotide sequence encoding an amino acid sequence” includes all nucleotide sequences that are degenerate forms of each other and encode the same amino acid sequence.

In certain exemplary embodiments, the nucleotide sequence encoding the chimeric antigen receptor described in the fifth aspect is selected from the group consisting of: (1) a sequence as set forth in SEQ ID NO: 84 or a degenerate variant thereof; (2) a sequence substantially identical to the sequence described in (1), for example, a sequence having a sequence identity of at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the sequence described in (1); or, a sequence having a substitution of one or more nucleotides as compared to the sequence described in (1); and the sequence substantially retains at least one biological activity (e.g., ability to encode a sequence capable of directing the specificity and reactivity of an immune effector cell to a MSLN-expressing cell in a non-MHC restricted manner) of the nucleotide sequence from which it was derived.

In certain exemplary embodiments, the nucleotide sequence encoding the chimeric antigen receptor described in the fifth aspect is selected from the group consisting of: (1) a sequence as set forth in SEQ ID NO: 89 or a degenerate variant thereof; (2) a sequence substantially identical to the sequence described in (1), for example, a sequence having a sequence identity of at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the sequence described in (1); or, a sequence having a substitution of one or more nucleotides as compared to the sequence described in (1); and the sequence substantially retains at least one biological activity (e.g., ability to encode a sequence capable of directing the specificity and reactivity of an immune effector cell to a MSLN-expressing cell in a non-MHC restricted manner) of the nucleotide sequence from which it was derived.

In certain exemplary embodiments, the nucleotide sequence encoding the chimeric antigen receptor described in the fifth aspect is selected from the group consisting of: (1) a sequence as set forth in SEQ ID NO: 91 or a degenerate variant thereof; (2) a sequence substantially identical to the sequence described in (1), for example, a sequence having a sequence identity of at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the sequence described in (1); or, a sequence having a substitution of one or more nucleotides as compared to the sequence described in (1); and the sequence substantially retains at least one biological activity (e.g., ability to encode a sequence capable of directing the specificity and reactivity of an immune effector cell to a MSLN-expressing cell in a non-MHC restricted manner) of the nucleotide sequence from which it was derived.

As described above, the CAR of the present invention can also be co-expressed with an additional biologically active molecule to exert a synergistic anti-tumor effect.

Accordingly, a seventh aspect of the present invention further provides a nucleic acid construct, which comprises a first nucleic acid sequence encoding the chimeric antigen receptor described in the fifth aspect, and further comprises a second nucleic acid sequence encoding an additional biologically active molecule.

In certain embodiments, the additional biologically active molecule encoded by the second nucleic acid sequence has anti-tumor activity.

In certain embodiments, the additional biologically active molecule encoded by the second nucleic acid sequence is one or more selected from the following components: immune checkpoint inhibitor (e.g., anti-PD-1, PD-L1, CTLA-4, or LAG-3 antibody or antigen-binding fragment thereof), cytokine (e.g., IL-15, IL-7, IL-12, IL-18, or IL-21), or membrane chimeric polypeptide (e.g., mIL-15, mIL-7, mIL-12, mIL-18, or mIL-21).

In certain embodiments, the additional biologically active molecule encoded by the second nucleotide sequence further comprises a signal peptide-2 at its N-terminal. In certain embodiments, the signal peptide-2 is different from the signal peptide contained in the chimeric antigen receptor encoded by the first nucleic acid sequence. In certain embodiments, the signal peptide-2 at the N-terminal of the additional biologically active molecule is an IL2 signal peptide, and the IL2 signal peptide refers to a signal peptide sequence contained in the IL2 native gene sequence, preferably, the IL2 native gene is a human IL2 native gene, the IL2 signal peptide is a human IL2 signal peptide. In certain exemplary embodiments, the IL2 signal peptide comprises the sequence as set forth in SEQ ID NO: 74.

In certain embodiments, the first nucleotide sequence is located at upstream of the second nucleotide sequence.

In certain embodiments, the first and second nucleic acid sequences are linked by a nucleotide sequence encoding a self-cleaving peptide (e.g., P2A, E2A, F2A, T2A, or any combination thereof). In certain embodiments, the self-cleaving peptide is P2A (e.g., the one as set forth in SEQ ID NO: 72). In certain exemplary embodiments, the sequence encoding the self-cleaving peptide is linked to the 3′ end of the first nucleotide sequence, and is linked to the 5′ end of the second nucleotide sequence.

In certain embodiments, the immune checkpoint inhibitor selected for the additional biologically active molecule is anti-PD-1 or PD-L1 antibody or antigen-binding fragment thereof (e.g., scFv).

In certain embodiments, the anti-PD-1 or PD-L1 antibody or antigen-binding fragment thereof comprises a heavy chain variable region and/or a light chain variable region of any one of the following groups: (1) a heavy chain variable region and/or a light chain variable region of Nivolumab or a variant thereof, (2) a heavy chain variable region and/or a light chain variable region of Pembrolizumab or a variant thereof, (3) a heavy chain variable region and/or a light chain variable region of Atezolizumab or a variant thereof, (4) a heavy chain variable region and/or a light chain variable region of Durvalumab or a variant thereof, (5) a heavy chain variable region and/or a light chain variable region of Avelumab or a variant thereof, (6) a VH having the sequence as set forth in SEQ ID NO: 79 or a variant thereof and/or a VL having the sequence as set forth in SEQ ID NO: 80 or a variant thereof. The variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% compared to the sequence from which it is derived, or has a substitution, deletion or addition of one or several amino acids (e.g., a substitution, deletion or addition of 1, 2, 3, 4, 5, 6, 7, 8, 9, or 10 amino acids) as compared to the sequence from which it is derived: preferably, the substitution is a conservative substitution.

In certain embodiments, the anti-PD-1 or PD-L1 antibody or antigen-binding fragment thereof is a single chain antibody (e.g., scFv).

In certain embodiments, the additional biologically active molecule comprises an anti-PD-1 single chain antibody, in which the anti-PD-1 single chain antibody has a VH with the sequence as set forth in SEQ ID NO: 79 and/or a VL with the sequence as set forth in SEQ ID NO: 80. In certain embodiments, the VH and VL are linked by a linker. In certain embodiments, the linker comprises a sequence of (GₘS)ₙ, wherein m is an integer selected from 1 to 6. In certain embodiments, m is 3, 4, or 5; and n is an integer selected from 1 to 10. In certain embodiments, n is 2, 3, 4, 5, or 6.

In certain embodiments, the anti-PD-1 single chain antibody comprises an amino acid sequence selected from the group consisting of: (1) an amino acid sequence as set forth in SEQ ID NO: 77: (2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence as set forth in SEQ ID NO: 77: (3) a sequence having a substitution, deletion or addition of one or several amino acids (e.g., a substitution, deletion or addition of 1, 2, 3, 4, 5, 6, 7, 8, 9, or 10 amino acids) as compared to the sequence as set forth in SEQ ID NO: 77: preferably, the substitution is a conservative substitution.

In certain exemplary embodiments, the nucleic acid construct of the seventh aspect comprises, in order from its 5′ end to its 3′ end: the first nucleic acid sequence, a nucleotide sequence encoding a self-cleaving peptide, a nucleotide sequence encoding a signal peptide-2, a nucleotide sequence encoding an immune checkpoint inhibitor. In certain exemplary embodiments, the nucleic acid construct of the seventh aspect comprises a nucleotide sequence selected from the group consisting of: (1) a nucleotide sequence as set forth in SEQ ID NO: 85 or a degenerate variant thereof; (2) a sequence substantially identical to the sequence described in (1), for example, a sequence having a sequence identity of at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the sequence described in (1).

In certain embodiments, the membrane-bound polypeptide selected for the additional biologically active molecule is mIL-15. In certain embodiments, the membrane-bound polypeptide mIL-15 comprises an amino acid sequence selected from the group consisting of: (1) a sequence as set forth in SEQ ID NO: 81: (2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence as set forth in SEQ ID NO: 81: (3) a sequence having a substitution, deletion or addition of one or several amino acids (e.g., a substitution, deletion or addition of 1, 2, 3, 4, 5, 6, 7, 8, 9, or 10 amino acids) as compared to the sequence as set forth in SEQ ID NO: 81: preferably, the substitution is a conservative substitution.

In certain exemplary embodiments, the nucleic acid construct of the seventh aspect comprises, in order from its 5′ end to its 3′ end: the first nucleic acid sequence, a nucleotide sequence encoding a self-cleaving peptide, a nucleotide sequence encoding a membrane-bound polypeptide. In certain exemplary embodiments, the nucleic acid construct of the seventh aspect comprises a nucleotide sequence selected from the group consisting of: (1) a nucleotide sequence as set forth in SEQ ID NO: 86 or a degenerate variant thereof; (2) a sequence substantially identical to the sequence described in (1), for example, a sequence having a sequence identity of at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% %, or 100% as compared to the sequence described in (1).

In certain embodiments, the additional biologically active molecule encoded by the second nucleic acid sequence comprises at least two components selected from the group consisting of: immune checkpoint inhibitor (e.g., anti-PD-1, PD-L1, CTLA-4, or LAG-3 antibody or antigen-binding fragment thereof), cytokine (e.g., IL-15, IL-7, IL-12, IL-18, or IL-21), or membrane-bound polypeptide (e.g., mIL-15, mIL-7, mIL-12, mIL-18, or mIL-21).

In certain embodiments, the nucleotide sequences encoding the at least two components contained in the second nucleic acid sequence are linked with each other via a nucleotide sequence encoding a self-cleaving peptide (e.g., P2A, E2A, F2A, T2A, or any combination thereof). In certain embodiments, the self-cleaving peptide is P2A (e.g., the one as set forth in SEQ ID NO: 72).

In certain embodiments, the additional biologically active molecule encoded by the second nucleic acid sequence comprises: (i) an anti-PD-1 antibody or antigen-binding fragment thereof (e.g., scFv), and (ii) mIL-15.

In certain exemplary embodiments, the nucleic acid construct of the seventh aspect comprises, in order from its 5′ end to its 3′ end, the first nucleic acid sequence, a nucleotide sequence encoding a self-cleaving peptide, a nucleotide sequence encoding a signal peptide-2, a nucleotide sequence encoding an anti-PD-1 antibody or antigen-binding fragment thereof, a nucleotide sequence encoding a self-cleaving peptide, and a nucleotide sequence encoding mIL-15. In certain exemplary embodiments, the nucleic acid construct of the seventh aspect comprises a nucleotide sequence selected from the group consisting of: (1) a nucleotide sequence as set forth in SEQ ID NO: 87 or a degenerate variant thereof; (2) a sequence substantially identical to the sequence described in (1), for example, a sequence having a sequence identity of at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the sequence described in (1).

An eighth aspect of the present invention provides a vector, which comprises the isolated nucleic acid molecule of the sixth aspect, or the nucleic acid construct of the seventh aspect.

In certain embodiments, the vector is selected from the group consisting of DNA vector, RNA vector, plasmid, transposon vector, CRISPR/Cas9 vector, viral vector.

In certain embodiments, the vector is an expression vector.

In certain embodiments, the vector is an episomal vector.

In certain embodiments, the vector is a viral vector.

In certain exemplary embodiments, the viral vector is a lentiviral, adenoviral, or retroviral vector.

In certain embodiments, the vector is an episomal or non-integrating viral vector, such as an integration-deficient retrovirus or lentivirus.

A ninth aspect of the present invention provides a host cell, which comprises the isolated nucleic acid molecule of the sixth aspect, the nucleic acid construct of the seventh aspect or the vector of the eighth aspect. The vector described above can be introduced into the host cell by various suitable means, such as calcium phosphate transfection, DEAE-dextran mediated transfection, microinjection, electroporation, TALEN method, ZFN method, non-viral vector mediated transfection (e.g., liposome) or viral vector-mediated transfection (e.g., lentiviral infection, retroviral infection, adenoviral infection), and other physical, chemical or biological methods for transfer into host cells, such as transposon technology, CRISPR-Cas9 and other technologies.

In certain embodiments, the host cell comprises the isolated nucleic acid molecule of the sixth aspect or a vector comprising the nucleic acid molecule, and the host cell expresses the chimeric antigen receptor of the present invention.

In certain embodiments, the host cell comprises the nucleic acid construct of the seventh aspect or a vector comprising the nucleic acid construct, and the host cell expresses the chimeric antigen receptor of the present invention and an additional biologically active molecule.

In certain embodiments, the host cell is selected from mammalian (e.g., human) immune cells. In certain embodiments, the immune cell is derived from a patient or healthy donor. In certain embodiments, the immune cell is selected from the group consisting of T lymphocyte, natural killer (NK) cell, monocyte, macrophage or dendritic cell, and any combination thereof; preferably, the immune cell is derived from T lymphocyte or NK cell.

A tenth aspect of the present invention provides a method for preparing a cell expressing the chimeric antigen receptor of the present invention, comprising: (1) providing a host cell; (2) introducing the isolated nucleic acid molecule according to the sixth aspect or a vector comprising the nucleic acid molecule into the host cell, to obtain a host cell capable of expressing the chimeric antigen receptor. Also provided is a method for preparing a cell co-expressing the chimeric antigen receptor of the present invention and an another biologically active molecule, comprising: (1) providing a host cell; (2) introducing the nucleic acid construct of the seventh aspect or a vector comprising the nucleic acid construct into the host cell, to obtain a host cell capable of co-expressing the chimeric antigen receptor and the additional biologically active molecule.

In certain embodiments, the host cell is selected from immune cell, such as T lymphocyte, NK cell, monocyte, dendritic cell, macrophage, and any combination thereof. In certain embodiments, the immune cell is selected from T lymphocyte, NK cell, monocyte, macrophages, or dendritic cell, and any combination of these cells.

In certain embodiments, in step (1), the immune cell is provided from a patient or a healthy donor and undergoes pretreatment: the pretreatment comprises sorting, activation and/or proliferation of the immune cell: in certain embodiments, the pretreatment comprises contacting the immune cell with an anti-CD3 antibody and an anti-CD28 antibody, thereby stimulating the immune cell and inducing its proliferation, thereby generating a pretreated immune cell.

In certain embodiments, in step (2), the nucleic acid molecule or vector is introduced into the immune cell by viral infection. In certain embodiments, in step (2), the nucleic acid molecule or vector is introduced into the immune cell by means of non-viral vector transfection, such as transposon-based vector system, CRISPR/Cas9 vector, TALEN method, ZFN method, electroporation method, calcium phosphate transfection, DEAE-dextran mediated transfection or microinjection, and so on.

In certain embodiments, after step (2), the method further comprises: expanding the immune cell obtained in step (2).

### Engineered Immune Cell

Immune cells derived from a patient or healthy donor can be transformed into immune cells expressing a CAR that specifically binds to MSLN and optionally additional biologically active molecule by the above-mentioned preparation method provided by the present invention.

Therefore, an eleventh aspect of the present invention further provides an engineered immune cell, which expresses the CAR of the present invention that specifically binds to MSLN.

In certain embodiments, the engineered immune cell comprises the isolated nucleic acid molecule of the sixth aspect or a vector comprising the nucleic acid molecule.

In certain embodiments, the engineered immune cell also expresses an additional biologically active molecule. In certain embodiments, the engineered immune cell comprises the nucleic acid construct of the seventh aspect or a vector comprising the nucleic acid construct. In some cases, while expressing the CAR described in the fifth aspect, the engineered immune cell of the present invention can also co-express an additional biologically active molecule, in which the chimeric antigen receptor that specifically binds to MSLN can become an independent CAR having an extracellular antigen-binding domain, a spacer domain, a transmembrane domain, and an intracellular signaling domain, while the additional biologically active molecule can be secreted extracellularly or expressed as a membrane-bound polypeptide or protein. With the expansion and enrichment of the immune cell expressing the CAR specifically binding to MSLN in the tumor microenvironment, the additional biologically active molecule is also enriched in the tumor microenvironment to exert synergistic anti-tumor effect with the anti-MSLN-CAR.

In certain embodiments, the immune cell is derived from T lymphocyte, NK cell, monocyte, macrophage, or dendritic cell, or any combination thereof, of a patient or healthy donor. These immune cells are prepared into engineered immune cells by introducing the isolated nucleic acid molecule described in the sixth aspect, the nucleic acid construct described in the seventh aspect or the vector described in the eighth aspect by the method provided in the tenth aspect.

In certain embodiments, the engineered immune cell may have a binding specificity for a target other than MSLN, for example: the engineered immune cell further expresses a CAR that is not specific for MSLN; preferably, the CAR that is not specific for MSLN has specificity for a target selected from the group consisting of: CD19, GPC3, PSMA, MUC1, EGFR, HER2, CD276, GD2, BCMA, CD33 or Claudin18.2.

In certain embodiments, the transcription or expression of one or both the target genes, i.e., the gene involved in the immune exclusion of engineered immune cells (e.g., TRAC, TRBC, B2M, HLA-A, HLA-B, or HLA-C) and the gene of immune co-inhibitory pathway or signaling molecule (e.g., PD-1, CTLA-4 or LAG-3), is inhibited such that the target gene-mediated signaling is blocked in the engineered immune cells; preferably, the method by which the transcription or expression of the target genes is inhibited is selected from the group consisting of gene knockout (e.g., CRISPR, CRISPR/Cas9), homologous recombination, and interfering RNA.

### Immune Cell Composition

In a twelfth aspect, the present invention further provides an immune cell composition, in which the immune cell composition comprises the aforementioned engineered immune cell, and optionally unengineered and/or unsuccessfully engineered immune cells, and these unengineered and/or unsuccessfully engineered immune cells do not express a CAR specific for MSLN. Limited to the current level of technology and for some unknown reasons, not all immune cells can be engineered to express a CAR specific for MSLN. Moreover, the immune cells that do not express CAR also have certain biological activities, so the immune cell composition can contain immune cells that express and do not express CAR specific for MSLN, and the immune cell composition can still meet the needs of clinical applications.

In certain embodiments, the engineered immune cell expressing a CAR specific for MSLN accounts for about 10% to 100%, preferably 40% to 80%, of the total cell number of the immune cell composition.

In certain embodiments, the immune cell composition is cultured into an immune cell line, thus, in another aspect, the present invention also provides an immune cell line comprising the immune cell composition.

In another aspect, the present invention provides a kit for the preparation of a chimeric antigen receptor that specifically binds to MSLN, or for the preparation of a cell expressing the chimeric antigen receptor or an immune cell co-expressing the chimeric antigen receptor and the additional biologically active molecule. In certain embodiments, the kit comprises the isolated nucleic acid molecule of the sixth aspect, the nucleic acid construct of the seventh aspect or the vector of the eighth aspect, or the host cell of the ninth aspect, and necessary solvent, such as sterile water, physiological saline, or cell culture medium, such as LB medium, or EliteCell Primary T Lymphocyte Culture System (Cat. No.: PriMed-EliteCell-024), and optionally, further comprises an instruction for use.

In another aspect, the present invention provides a use of the aforementioned kit for preparing a chimeric antigen receptor capable of specifically binding to MSLN, or a cell expressing the chimeric antigen receptor, or an immune cell co-expressing the chimeric antigen receptor and the additional biologically active molecule.

### Pharmaceutical Composition

In a thirteenth aspect, the present invention provides a pharmaceutical composition, which comprises the antibody or antigen-binding fragment thereof described in the first aspect of the present invention, the chimeric antigen receptor of the fifth aspect (including the bispecific chimeric antigen receptor, or the CAR construct co-expressed with an additional biologically active molecule), the isolated nucleic acid molecule of the second aspect or the sixth aspect, the nucleic acid construct of the seventh aspect, the vector of the third aspect or the eighth aspect, the host cell of the fourth aspect or the ninth aspect, the engineered immune cell of the eleventh aspect, or the immune cell composition of the twelfth aspect, and a pharmaceutically acceptable carrier and/or excipient.

In certain embodiments, the pharmaceutical composition further comprises an additional pharmaceutically active agent, such as a drug with anti-tumor activity (e.g., anti-PD1 antibody, anti-PD-L1 antibody, anti-CTLA-4 antibody, pemetrexed, cisplatin, paclitaxel, gemcitabine, capecitabine, or FOLFIRINOX).

In certain embodiments, the antibody or antigen-binding fragment thereof of the first aspect, the chimeric antigen receptor of the fifth aspect, the isolated nucleic acid molecule of the second aspect or the sixth aspect, the nucleic acid construct of the seventh aspect, the vector of the third aspect or the eighth aspect, the host cell of the fourth aspect or the ninth aspect, the engineered immune cell of the eleventh aspect, or the immune cell composition of the twelfth aspect of the present invention and the additional pharmaceutically active agent may be administered simultaneously, separately or sequentially.

In certain embodiments, the pharmaceutical composition of the present invention comprises: the isolated nucleic acid molecule of the sixth aspect, the nucleic acid construct of the seventh aspect or the vector of the eighth aspect, or the host cell of the ninth aspect.

In certain embodiments, the pharmaceutical composition of the present invention comprises: the engineered immune cell or the immune cell composition of the present invention.

In the present invention, the antibody or antigen-binding fragment thereof described in the first aspect, the chimeric antigen receptor described in the fifth aspect, the isolated nucleic acid molecule described in the second aspect or the sixth aspect, the nucleic acid construct described in the seventh aspect, the vector described in the third aspect or the eighth aspect, the host cell described in the fourth aspect or the ninth aspect, the engineered immune cell described in the eleventh aspect or the immune cell composition described in the twelfth aspect may be formulated into any dosage form known in the medical art, for example, tablet, pill, suspension, emulsion, solution, gel, capsule, powder, granule, elixir, lozenge, suppository, injection (including solution for injection, sterile powder for injection, and concentrated solution for injection), inhalation, spray, etc. The preferred dosage form depends on the intended mode of administration and therapeutic use. The pharmaceutical composition of the present invention should be sterile and stable under the conditions of manufacture and storage. A preferred dosage form is an injection. Such injection can be sterile solution for injection. In addition, sterile solution for injection can be prepared as a sterile lyophilized powder (e.g., by vacuum drying or freeze-drying) for ease of storage and use. Such sterile lyophilized powder can be dispersed in a suitable vehicle such as water for injection (WFI), bacteriostatic water for injection (BWFI), sodium chloride solution (e.g., 0.9% (w/v) NaCl), glucose solution (e.g., 5% glucose), surfactant-containing solution (e.g., 0.01% polysorbate-20), pH buffered solution (e.g., phosphate buffered solution), Ringer's solution, and any combination thereof.

In the present invention, the antibody or antigen-binding fragment thereof described in the first aspect, the chimeric antigen receptor described in the fifth aspect, the isolated nucleic acid molecule described in the second aspect or the sixth aspect, the nucleic acid construct described in the seventh aspect, the vector described in the third aspect or the eighth aspect, the host cell described in the fourth aspect or the ninth aspect, the engineered immune cell described in the eleventh aspect or the immune cell composition described in the twelfth aspect can be administered by any suitable method known in the art, including, but not limited to, oral, buccal, sublingual, ocular, topical, parenteral, rectal, intrathecal, intracytoplasmic reticulum, inguinal, intravesical, topical (e.g., powder, ointment, or drop), or nasal administration route. However, for many therapeutic uses, the preferred administration route/mode is parenteral administration (e.g., intravenous or bolus injection, subcutaneous injection, intraperitoneal injection, intramuscular injection). The skilled in the art will appreciate that the route and/or mode of administration will vary depending on the intended purpose. In certain embodiments, the antibody or antigen-binding fragment thereof of the first aspect, the chimeric antigen receptor of the fifth aspect, the isolated nucleic acid molecule of the second aspect or the sixth aspect, the nucleic acid construct of the seventh aspect, the vector of the third aspect or the eighth aspect, the host cell of the fourth aspect or the ninth aspect, the engineered immune cell of the eleventh aspect, or the immune cell composition of the twelfth aspect of the present invention is administered by intravenous injection or bolus injection.

The pharmaceutical composition of the present invention may comprise a “therapeutically effective amount” or “prophylactically effective amount” of the antibody or antigen-binding fragment thereof described in the first aspect of the present invention, the chimeric antigen receptor described in the fifth aspect, the isolated nucleic acid molecule described in the second aspect or the sixth aspect, the nucleic acid construct described in the seventh aspect, the vector described in the third aspect or the eighth aspect, the host cell described in the fourth aspect or the ninth aspect, the engineered immune cell as described in the eleventh aspect or the immune cell composition described in the twelfth aspect. The “prophylactically effective amount” refers to an amount sufficient to prevent, stop, or delay the onset of a disease. The “therapeutically effective amount” refers to an amount sufficient to cure or at least partially arrest a disease and its complications in a patient already suffering from the disease. In the present invention, for the antibody or antigen-binding fragment thereof described in the first aspect, the chimeric antigen receptor described in the fifth aspect, the isolated nucleic acid molecule described in the second aspect or the sixth aspect, the nucleic acid construct described in the seventh aspect, the vector described in the third aspect or the eighth aspect, the host cell described in the fourth aspect or the ninth aspect, the engineered immune cell described in the eleventh aspect or the immune cell composition described in the twelfth aspect, its therapeutically effective amount may vary depending on factors such as the severity of the disease to be treated, the general state of the patient's own immune system, the patient's general conditions such as age, weight and sex, the administration mode of drug, and other concurrently administered therapy, etc.

### Therapeutic Method and Use

In another aspect, the present invention provides a method for preventing and/or treating a disease associated with mesothelin expression in a subject (e.g., a human), the method comprising administering to the subject in need thereof an effective amount of the antibody or antigen-binding fragment thereof described in the first aspect of the present invention, the chimeric antigen receptor described in the fifth aspect, the isolated nucleic acid molecule described in the second aspect or the sixth aspect, the nucleic acid construct described in the seventh aspect, the vector described in the third aspect or the eighth aspect, the host cell described in the fourth aspect or the ninth aspect, the engineered immune cell described in the eleventh aspect or the immune cell composition described in the twelfth aspect, or the pharmaceutical composition.

In certain embodiments, the disease associated with mesothelin expression is selected from a proliferative disease, such as a tumor. In certain embodiments, the disease associated with mesothelin expression is a non-tumor-related condition associated with mesothelin expression.

In certain embodiments, the tumor is an MSLN-positive tumor. In certain embodiments, the tumor is selected from solid tumor (e.g., malignant pleural mesothelioma, pancreatic cancer, lung cancer (e.g., lung squamous carcinoma), breast cancer, ovarian cancer (e.g., epithelial ovarian cancer)).

In certain embodiments, the method comprises administering to the subject an effective amount of the antibody or antigen-binding fragment thereof described in the first aspect.

In certain embodiments, the method comprises administering to the subject an effective amount of the chimeric antigen receptor of the fifth aspect, the isolated nucleic acid molecule of the sixth aspect, the nucleic acid construct of the seventh aspect, the vector of the eighth aspect, the host cell of the ninth aspect, the engineered immune cell of the eleventh aspect, or the immune cell composition of the twelfth aspect. In certain embodiments, the host cell is an immune cell (e.g., human immune cell).

In certain embodiments, the method comprises the steps of: (1) providing an immune cell (e.g., T lymphocyte, NK cell, monocyte, macrophage, dendritic cell, or any combination thereof) required by the subject: (2) introducing the isolated nucleic acid molecule described in the sixth aspect of the present invention, the nucleic acid construct described in the seventh aspect or the vector described in the eighth aspect into the immune cell of step (1), to obtain a cell expressing the chimeric antigen receptor and optionally the additional biologically active molecule: (3) administering the immune cell obtained in step (2) to the subject for treatment.

In certain embodiments, the method comprises administering to the subject the immune cell expressing the CAR of the present invention in divided doses, for example, in one, two, three or more divided doses, for example, administering a first percentage of the total dose on the first treatment day, and administering a second percentage of the total dose on a subsequent (e.g., second, third, fourth, fifth, sixth, or seventh or later) treatment day, for example, administering a third percentage of the total dose (e.g., the remaining percentage) on a subsequent (e.g., third, fourth, fifth, sixth, seventh, eighth, ninth, tenth, or later) treatment day.

In certain embodiments, 10% of the total dose of cells is administered on the first treatment day, 30% of the total dose of cells is administered on the second day, and the remaining 60% of the total dose of cells is administered on the third day.

In certain embodiments, 50% of the total dose of cells is administered on the first treatment day, and 50% of the total dose of cells is administered on a subsequent (e.g., second, third, fourth, fifth, sixth, or seventh or later) treatment day. In certain embodiments, ⅓ of the total dose of cells is administered on the first treatment day, ⅓ of the total dose of cells is administered on a subsequent (e.g., second, third, fourth, fifth, sixth, or seventh or later) treatment day, and ⅓ of the total dose is administered on a subsequent (e.g., third, fourth, fifth, sixth, seventh, eighth, ninth, tenth, or later) treatment day.

In certain embodiments, the total dose of cells comprises 1×10⁷ to or 10×10⁸ CAR-positive immune cells, for example, comprises (1-5)×10⁷ to (5-10)×10⁸ CAR-positive immune cells.

In certain embodiments, the physician may adjust the dosage or treatment regimen based on the patient's state, the size and stage of tumor, or clinical circumstances such as the drug being used in combination therapy.

In certain embodiments, the antibody or antigen-binding fragment thereof of the first aspect of the present invention, the chimeric antigen receptor of the fifth aspect, the isolated nucleic acid molecule of the second aspect or the sixth aspect, the nucleic acid construct of the seventh aspect, the vector of the third aspect or the eighth aspect, the host cell of the fourth aspect or the ninth aspect, the engineered immune cell of the eleventh aspect or the tenth aspect, the immune cell composition of the twelfth aspect or the pharmaceutical composition is administered in combination with an additional agent. In certain embodiments, the additional agent comprises: (i) an agent capable of increasing a function of a cell comprising the CAR nucleic acid or the CAR polypeptide (e.g., an immune cell expressing the CAR of the present invention, the engineered immune cell or immune cell composition of the present invention): (ii) an agent capable of ameliorating one or more side-effects associated with the administration of a cell comprising the CAR nucleic acid or the CAR polypeptide (e.g., an immune cell expressing the CAR of the present invention, the engineered immune cell or immune cell composition of the present invention); (iii) an additional pharmaceutically active agent with antitumor activity. These reagents can be used before, at the same time or after the administration of the antibody or antigen-binding fragment thereof of the first aspect of the present invention, the chimeric antigen receptor of the fifth aspect, the isolated nucleic acid molecule of the second aspect or the sixth aspect, the nucleic acid construct of the seventh aspect, the vector of the third aspect or the eighth aspect, the host cell of the fourth aspect or the ninth aspect, the engineered immune cell of the eleventh aspect, the immune cell composition of the twelfth aspect or the pharmaceutical composition.

In certain embodiments, the method described above further comprise administering to the subject a second therapy, in which the second therapy may be any therapy known for use in tumor, such as surgery, chemotherapy, radiation therapy, immunotherapy, gene therapy, DNA therapy, RNA therapy, nanotherapy, viral therapy, adjuvant therapy, and any combination thereof.

In certain embodiments, the second therapy may be administered separately or in combination with the method described above: alternatively, the second therapy may be administered concurrently or sequentially with the method described above.

In certain embodiments, the subject may be a mammal, such as a human.

In another aspect, the present invention provides a use of the antibody or antigen-binding fragment thereof of the first aspect of the present invention, the chimeric antigen receptor of the fifth aspect, the isolated nucleic acid molecule of the second aspect or the sixth aspect, the nucleic acid construct of the seventh aspect, the vector of the third aspect or the eighth aspect, the host cell of the fourth aspect or the ninth aspect, the engineered immune cell of the eleventh aspect, the immune cell composition of the twelfth aspect or the pharmaceutical composition in the manufacture of a medicament for preventing and/or treating a tumor. The dosage, dosage form, administration route, indication, combination therapy and other aspects of the aforementioned therapeutic method can be applied to the use of the medicament.

### Abbreviations

| | |
|---|---|
| CAR | Chimeric antigen receptor |
| CDR | Complementarity determining region |
| | in immunoglobulin variable region |
| CDR-H1 | Complementarity determining region 1 in |
| | immunoglobulin heavy chain variable region |
| CDR-H2 | Complementarity determining region 2 in |
| | immunoglobulin heavy chain variable region |
| CDR-H3 | Complementarity determining region 3 |
| | in immunoglobulin heavy chain variable region |
| CDR-L1 | Complementarity determining region 1 |
| | in immunoglobulin light chain variable region |
| CDR-L2 | Complementarity determining region 2 in |
| | immunoglobulin light chain variable region |
| CDR-L3 | Complementarity determining region 3 in |
| | immunoglobulin light chain variable region |
| FR | Antibody framework region: amino acid residues |
| | other than CDR residues in antibody variable regions |
| VH | Antibody heavy chain variable region |
| VL | Antibody heavy light variable region |
| Kabat | Immunoglobulin alignment and numbering |
| | system proposed by Elvin A. |
| | Kabat (see, for example, Kabat et al., |
| | Sequences of Proteins of |
| | Immunological Interest, 5th Ed. Public |
| | Health Service, National Institutes |
| | of Health, Bethesda, Md., 1991). |
| IMGT | Numbering system based on the |
| | international ImMunoGeneTics |
| | information system ® (IMGT) initiated by |
| | Lefranc et al., see, Lefranc et al., |
| | Dev. Comparat. Immunol. 27: 55-77, 2003. |
| Chothia | Immunoglobulin numbering system proposed |
| | by Chothia et al., which is a classical |
| | rule for identifying CDR region |
| | boundaries based on the location |
| | of structural loop regions (see, for example, |
| | Chothia & Lesk (1987) J. Mol. Biol. 196: 901-917; |
| | Chothia et al. (1989) Nature 342: 878-883). |
| IL-2 | Interleukin 2 |
| IFN | Interferon |
| PCR | Polymerase chain reaction |
| FACS | Fluorescence activated cell sorting |
| K_{D} | Equilibrium dissociation constant |
| kon | Association rate constant |
| kdis | Dissociation rate constant |

### Definition of Terms

In the present invention, unless otherwise specified, scientific and technical terms used herein have the meanings commonly understood by those skilled in the art. In addition, the procedures of molecular biology, microbiology, cell biology, biochemistry, immunology and the like used herein are all routine steps widely used in the corresponding fields. Meanwhile, for a better understanding of the present invention, definitions and explanations of related terms are provided below.

As used herein, the term “antibody” refers to an immunoglobulin molecule capable of specifically binding to a target (e.g., carbohydrate, polynucleotide, lipid, polypeptide, etc.) through at least one antigen recognition site located in the variable region of the immunoglobulin molecule. As used herein, the term comprises not only intact polyclonal or monoclonal antibody, but also fragment thereof (e.g., Fab, Fab′, F(ab′)₂, Fv), single chain antibody (e.g., scFv, di-scFv, (scFv)₂) and domain antibody (including, for example, shark and camel antibodies), as well as fusion protein including antibody, and immunoglobulin molecule in any other modified form comprising antigen recognition site. The antibody of the present invention is not limited by any particular method for producing the antibody. The antibody includes any type of antibody, such as IgG, IgA, or IgM (or a subclass thereof), and the antibody needs not be of any particular type. Depending on the amino acid sequence of the antibody heavy chain constant region, immunoglobulins can be assigned to different types. There are five main types of immunoglobulins: IgA, IgD, IgE, IgG, and IgM, several of which can be further divided into subclasses (isotypes), such as IgG1, IgG2, IgG3, IgG4, IgA1, and IgA2. The heavy chain constant regions corresponding to the different types of immunoglobulins are called α, δ, ε, γ, and μ, respectively. The antibody light chains can be classified as κ (kappa) and λ (lambda) light chains. The subunit structures and three-dimensional configurations of different types of immunoglobulins are well known. The heavy chain constant region consists of 4 domains (CH1, hinge region, CH2 and CH3). Each light chain consists of a light chain variable region (VL) and a light chain constant region (CL). The light chain constant region consists of one domain. CL. The constant domains are not directly involved in the binding of antibody to antigen, but exhibit a variety of effector functions, such as mediating the binding of immunoglobulin to a host tissue or factor, including various cells of immune system (e.g., effector cells) and the first component (C1q) of classical complement system.

The VH and VL regions of antibody can also be subdivided into regions of high variability (called as complementarity determining regions (CDRs)), interspersed with more conserved regions called as framework regions (FRs). Each V_{H} and V_{L} consists of 3 CDRs and 4 FRs arranged from amino-terminal to carboxy-terminal in the following order: FR1, CDR1, FR2, CDR2, FR3, CDR3, FR4. The variable regions (VH and VL) of each heavy chain/light chain pair respectively form the antigen binding site. The assignment of amino acids to regions or domains can follow the definitions of Kabat. Sequences of Proteins of Immunological Interest (National Institutes of Health. Bethesda. Md. (1987 and 1991)), or Chothia & Lesk (1987) J. Mol. Biol. 196:901-917: Chothia et al. (1989) Nature 342:878-883.

As used herein, the term “complementarity determining region” or “CDR” refers to the amino acid residues in the variable region of an antibody that are responsible for antigen binding. The variable regions of the heavy chain and light chain each contain three CDRs, designated as CDR1, CDR2 and CDR3. The precise boundaries of these CDRs can be defined by various numbering systems known in the art, for example according to the Kabat numbering system (Kabat et al., Sequences of Proteins of Immunological Interest. 5th Ed. Public Health Service. National Institutes of Health, Bethesda, Md., 1991), the Chothia numbering system (Chothia & Lesk (1987) J. Mol. Biol. 196:901-917: Chothia et al. (1989) Nature 342:878-883) or the IMGT numbering system (Lefranc et al., Dev. Comparat. Immunol. 27:55-77, 2003). For a given antibody, those skilled in the art will readily identify the CDRs defined by each numbering system. Also, correspondence between different numbering systems is well known to those skilled in the art (see, for example. Lefranc et al., Dev. Comparat. Immunol. 27:55-77, 2003).

In the present invention, the CDRs contained by the antibody or antigen-binding fragment thereof can be determined according to various numbering systems known in the art. In certain embodiments, the CDRs contained by the antibody or antigen-binding fragment thereof of the present invention are preferably determined by the Kabat, Chothia or IMGT numbering system.

As used herein, the term “framework region” or “FR” residues refers to those amino acid residues in the variable region of an antibody other than the CDR residues as defined above.

As used herein, the term “antigen-binding fragment” of antibody refers to a polypeptide of an antibody fragment, such as a polypeptide of a fragment of a full-length antibody, that retains the ability to specifically bind the same antigen to which the full-length antibody binds, and/or compete with the full-length antibody for specific binding to the antigen, which is also referred to as an “antigen-binding portion.” See generally, Fundamental Immunology, Ch. 7 (Paul. W., ed., 2nd ed., Raven Press, N.Y. (1989), which is hereby incorporated by reference in its entirety for all purposes. The antigen-binding fragment of antibody can be generated by recombinant DNA techniques or by enzymatic or chemical cleavage of an intact antibody. Non-limiting examples of antigen-binding fragments include camelid Ig, Ig NAR, Fab fragment, Fab′ fragment, F(ab′)₂ fragment, F(ab′)₃ fragment, Fd, Fv, scFv, di-scFv, (scFv) 2, minibody, bifunctional antibody, trifunctional antibody, tetrafunctional antibody, disulfide stabilized Fv protein (“dsFv”) and single domain antibody (sdAbs, nanobody), and such polypeptides comprising at least a portion of an antibody sufficient to confer specific antigen-binding ability to the polypeptide. The engineered antibody variants are reviewed by Holliger et al., 2005; Nat Biotechnol, 23: 1126-1136.

As used herein, the term “camelid Ig” or “camel VHH” refers to the smallest known antigen-binding unit of a heavy chain antibody (Koch-Nolte et al., FASEB J., 21: 3490-3498 (2007)). “Heavy chain antibody” or “camel antibody” refers to an antibody containing two VH domains and no light chain (Riechmann L, et al., J. Immunol. Methods. 231:25-38 (1999): WO94/04678; WO94/25591; U.S. Pat. No. 6,005,079).

As used herein, the term “IgNAR” or “immunoglobulin new antigen receptor” refers to a class of antibodies from the shark immune repertoire and consisting of homodimer of one variable new antigen receptor (VNAR) domain and five constant new antigen receptor (CNAR) domains.

As used herein, the term “Fd” refers to an antibody fragment consisting of VH and CH1 domains: the term “dAb fragment” refers to an antibody fragment consisting of VH domain (Ward et al., Nature, 341:544546 (1989)): the term “Fab fragment” refers to an antibody fragment consisting of VL. VH. CL and CH1 domains: the term “F(ab′)₂ fragment” refers to an antibody fragment comprising two Fab fragments that are linked by disulfide bridge on the hinge region; the term “Fab′ fragment” refers to a fragment that is obtained by reducing the disulfide bond linking two heavy chain fragments in an F(ab′)₂ fragment, and consists of one intact light chain and a heavy chain Fd fragment (consisting of VH and CH1 domains).

As used herein, the term “Fv” refers to an antibody fragment consisting of the VL and VH domains of one-arm of an antibody. Fv fragments are generally considered to be the smallest antibody fragments capable of forming a complete antigen-binding site. It is generally believed that the six CDRs confer antigen-binding specificity to an antibody. However, even one variable region (e.g., an Fd fragment, which contains only three antigen-specific CDRs) is able to recognize and bind an antigen, albeit with possibly lower affinity than the intact binding site.

As used herein, the term “Fc” refers to an antibody fragment formed by linking the second and third constant regions of a first heavy chain of antibody to the second and third constant regions of a second heavy chain through disulfide bonds. The Fc fragment of an antibody has many different functions, but is not involved in antigen binding.

As used herein, the term “scFv” refers to a single polypeptide chain comprising VL and VH domains, wherein the VL and VH are connected by a linker (see, for example. Bird et al., Science, 242:423-426 (1988): Huston et al., Proc. Natl. Acad. Sci. USA 85:5879-5883 (1988); and Pluckthun. The Pharmacology of Monoclonal Antibodies, Vol. 113, Eds. Roseburg and Moore, Springer-Verlag. New York, pp. 269-315 (1994)). Such scFv molecules can have the general structure: NH₂-VL-linker-VH-COOH or NH₂-VH-linker-VL-COOH. Suitable prior art linkers consist of repeated amino acid sequence of GGGGS or variants thereof. For example, a linker with the amino acid sequence (GGGGS)₄ can be used, while variants thereof are also usable (Holliger et al. (1993), Proc. Natl. Acad. Sci. USA 90:6444-6448). Other linkers useful in the present invention are described by Alfthan et al. (1995), Protein Eng. 8:725-731: Choi et al. (2001), Eur. J. Immunol. 31:94-106; Hu et al. (1996), Cancer Res. 56:3055-3061: Kipriyanov et al. (1999), J. Mol. Biol. 293:41-56; and Roovers et al. (2001). Cancer Immunol. In some cases, a disulfide bond may also exist between the VH and VL of scFv. In certain embodiments, the VH and VL domains can be positioned relative to each other in any suitable arrangement. Example includes scFv of NH₂-VH-VH-COOH, or NH₂-VL-VL-COOH. The scFv can form any engineering possible structure: single chain antibody (scFv), tandem single chain antibody (tandem di-scFvs), bifunctional antibody, trifunctional antibody, tetrafunctional antibody, disulfide-stabilized Fv protein, camel Ig, IgNAR, etc. In certain embodiments of the present invention, the scFv can form a di-scFv, which refers to an antibody formed by two or more individual scFvs in tandem connection. In certain embodiments of the present invention, the scFv can form (scFv) 2, which refers to an antibody formed by two or more individual scFvs in parallel connection.

As used herein, the term “bifunctional antibody” refers to an antibody fragment having two antigen-binding sites, in which the fragment comprises a heavy chain variable domain (VH) that is linked to a light chain variable domain (VL) in the same polypeptide chain (VH-VL). By using a linker that is too short to allow pairing between the two domains on the same chain, the domains are forced to pair with the complementary domains of the other chain, and two antigen binding sites are generated. The bifunctional antibody can be bivalent or bispecific. Bifunctional antibodies are more fully described, for example, in EP 404,097; WO 1993/01161; Hudson et al, Nat. Med., 9:129-134 (2003); and Hollinger et al, PNAS USA 90:6444-6448 (1993). Trifunctional antibodies and tetrafunctional antibodies are also described in Hudson et al., Nat. Med., 9:129-134 (2003).

As used herein, the term “single-domain antibody (sdAb)” has the meaning commonly understood by those of skill in the art, which refers to an antibody fragment composed of a single monomeric variable antibody domain (e.g., a single heavy chain variable region), which retains the ability to specifically bind to the same antigen bound by the full-length antibody (Holt, L, et al., Trends in Biotechnology, 21 (11): 484-490). Single-domain antibody is also known as nanobody.

Each of the aforementioned antibody fragments retains the ability to specifically bind to the same antigen bound by the full-length antibody, and/or compete with the full-length antibody for specific binding to the antigen.

An antigen-binding fragment of antibody (e.g., an antibody fragment described above) can be obtained from a given antibody (e.g., the antibody provided by the present invention) by using conventional techniques known to those of skill in the art (e.g., recombinant DNA techniques or enzymatic or chemical fragmentation methods), and the antigen-binding fragment of antibody is screened for specificity in the same manner as being used for the intact antibody.

Herein, unless the context clearly dictates otherwise, when the term “antibody” is referred to, it includes not only the intact antibody but also the antigen-binding fragments of the antibody.

As used herein, the expression “specifically binding” or “specific for” refers to a non-random binding reaction between two molecules, such as a reaction between an antibody and an antigen to which it targets. The strength or affinity of a specific binding interaction can be expressed in terms of the equilibrium dissociation constant (K_{D}) for that interaction. In the present invention, the term “K_{D}” refers to the dissociation equilibrium constant of a specific antibody-antigen interaction, which is used to describe the binding affinity between an antibody and an antigen. The smaller the equilibrium dissociation constant, the tighter the antibody-antigen binding and the higher the affinity between the antibody and the antigen.

The specific binding property between two molecules can be determined by using methods well known in the art. One of the methods involves measuring the rate of formation and dissociation of antigen binding site/antigen complex. Both the “association rate constant” (ka or kon) and the “dissociation rate constant” (kdis or koff) can be calculated from the concentrations and the actual rates of association and dissociation (see, Malmqvist M, Nature, 1993, 361:186-187). The ratio of kdis/kon is equal to the dissociation constant K_{D} (see, Davies et al., Annual Rev Biochem, 1990; 59:439-473). The values of K_{D}, kon and kdis can be measured by any valid methods. In certain embodiments, the dissociation constant can be measured in Biacore using surface plasmon resonance (SPR). In addition, bioluminescence interferometry or Kinexa can be used to measure the dissociation constant.

As used herein, the term “identity” refers to the match degree between two polypeptides or between two nucleic acids. When two sequences for comparison have the same monomer sub-unit of base or amino acid at a certain site (e.g., each of two DNA molecules has an adenine at a certain site, or each of two polypeptides has a lysine at a certain site), the two molecules are identical at the site. The percent identity between two sequences is a function of the number of identical sites shared by the two sequences over the total number of sites for comparison×100. For example, if 6 of 10 sites of two sequences are matched, these two sequences have an identity of 60%. For example, DNA sequences: CTGACT and CAGGTT share an identity of 50% (3 of 6 sites are matched). Generally, the comparison of two sequences is conducted in a manner to produce maximum identity. Such alignment can be conducted by using a computer program such as Align program (DNAstar, Inc.) which is based on the method of Needleman, et al. (J. Mol. Biol. 48:443-453, 1970). The percent identity between two amino acid sequences can also be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percentage of identity between two amino acid sequences can be determined by the algorithm of Needleman and Wunsch (J. Mol. Biol. 48:444-453 (1970)) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

As used herein, the term “conservative substitution” refers to an amino acid substitution that does not adversely affect or alter the intended properties of the protein/polypeptide comprising the amino acid sequence. For example, a conservative substitution can be introduced by standard techniques known in the art such as site-directed mutagenesis and PCR-mediated mutagenesis. Conservative amino acid substitutions include substitutions of amino acid residues with the ones that have similar side chains, for example, substitutions with residues that are physically or functionally similar to the corresponding amino acid residues (e.g., have similar size, shape, charge, chemical properties, including the ability to form covalent bond or hydrogen bond, etc.). Families of amino acid residues with similar side chains have been defined in the art. These families include amino acids with basic side chains (e.g., lysine, arginine, and histidine), acidic side chains (e.g., aspartic acid, glutamic acid), uncharged polar side chains (e.g., glycine, asparagine, glutamine, serine, threonine, tyrosine, cysteine, tryptophan), non-polar side chains (e.g., alanine, valine, leucine, isoleucine, proline, phenylalanine, methionine), β branched side chains (e.g., threonine, valine, isoleucine), and aromatic side chains (e.g., tyrosine, phenylalanine, tryptophan, histidine). Therefore, it is preferred to substitute the corresponding amino acid residue with another amino acid residue from the same side chain family. Methods for identifying conservative substitutions of amino acids are well known in the art (see, for example, Brummell et al., Biochem. 32:1180)-1187 (1993): Kobayashi et al. Protein Eng. 12 (10): 879-884 (1999) and Burks et al. Proc. Natl Acad. Set USA 94:412-417 (1997), which is incorporated herein by reference).

The twenty conventional amino acids involved herein are expressed in routine manners. See, for example, Immunology—A Synthesis (2nd Edition, E. S. Golub and D. R. Gren, Eds., Sinauer Associates, Sunderland, Mass. (1991)), which is incorporated herein by reference. In the present invention, the terms “polypeptide” and “protein” have the same meaning and are used interchangeably. And in the present invention, amino acids are generally represented by one-letter or three-letter abbreviations well known in the art. For example, alanine can be represented by A or Ala.

As used herein, the term “vector” refers to a nucleic acid delivery vehicle into which a polynucleotide can be inserted. The vector may include sequences that replicate directly and autonomously in the cell, or may include sequences sufficient to allow integration into the DNA of the host cell. When the vector can express the protein encoded by the inserted polynucleotide, the vector is called an expression vector. The vector can be introduced into a host cell by transformation, transduction or transfection, so that the genetic material elements carried by it can be expressed in the host cell. The vector is well known to those skilled in the art and includes, but is not limited to: plasmid: phagemid: cosmid: artificial chromosome such as yeast artificial chromosome (YAC), bacterial artificial chromosome (BAC) or P1 derived artificial chromosome (PAC); phage such as λ phage or M13 phage and viral vector. Non-limiting examples of viral vector include, retrovirus (including lentivirus), adenovirus, adeno-associated virus, herpesvirus (e.g., herpes simplex virus), poxvirus, baculovirus, papillomavirus, papovavirus (e.g., SV40). A vector may contain a variety of elements that control expression, including, but not limited to, promoter sequence, transcription initiation sequence, enhancer sequence, selection element, and reporter gene. Additionally, the vector may also contain an origin of replication site.

As used herein. “episomal” in the term “episomal vector” means that the vector is capable of replicating without integrating into the chromosomal DNA of the host and will not be gradually lost in dividing host cells, and it also means that the vector is extrachromosomally or episomally replicated.

As used herein, the term “viral vector” is used broadly to refer to a nucleic acid molecule (e.g., a transfer plasmid) that includes a virus-derived nucleic acid element that typically facilitates transfer or integration of the nucleic acid molecule into the genome of a cell, or a virus particle that mediates nucleic acid transfer. In addition to nucleic acids, viral particles will typically include various viral components, and sometimes host cell components.

The term “viral vector” can refer to a virus or viral particle capable of transferring a nucleic acid into a cell, or to the transferred nucleic acid itself. The viral vector and transfer plasmid contain structural and/or functional genetic elements derived primarily from the virus.

As used herein, the term “retroviral vector” refers to a viral vector or plasmid containing structural and functional genetic elements or portions thereof derived primarily from a retrovirus.

As used herein, the term “lentiviral vector” refers to a viral vector or plasmid containing structural and functional genetic elements or portions thereof (including LTR) derived primarily from a lentivirus. In certain embodiments, the terms “lentiviral vector”, “lentiviral expression vector” may be used to refer to a lentiviral transfer plasmid and/or infectious lentiviral particle. Where elements (e.g., cloning site, promoter, regulatory element, heterologous nucleic acid, etc.) are referred to herein, it should be understood that the sequences of these elements are present in the lentiviral particle of the present invention in RNA form, and in the DNA plasmid of the present invention in DNA form.

As used herein, an “integration-deficient” retrovirus or lentivirus refers to a retrovirus or lentivirus that has an integrase that is unable to integrate the viral genome into the genome of a host cell. In certain embodiments, the integrase protein is mutated to specifically reduce its integrase activity. The integration-deficient lentiviral vector can be obtained by modifying the pol gene encoding an integrase protein to generate a mutant pol gene encoding an integration-deficient integrase. The integration-deficient viral vectors have been described in patent application WO 2006/010834, which is incorporated herein by reference in its entirety.

As used herein, the term “host cell” refers to a cell that can be used for introduction of a vector, including, but not limited to, prokaryotic cell such as Escherichia coli or Bacillus subtilis, fungal cell such as yeast cell or Aspergillus, insect cell such as S2 fruit fly cell or Sf9), or animal cell such as fibroblast. CHO cell, COS cell, NSO cell, HeLa cell, BHK cell, HEK 293 cell or human cell, immune cell (e.g., T lymphocyte, NK cell, monocyte, macrophage or dendritic cell, etc.). The host cell can include a single cell or a population of cells.

As used herein, the term “chimeric antigen receptor” or “CAR” refers to a recombinant polypeptide construct comprising at least one antigen-binding domain, a spacer domain, a transmembrane domain, and a cytoplasmic signaling domain (also referred to herein as an “intracellular signaling domain”), which combine the antibody-based specificity for an antigen of interest (e.g., MSLN) with an activating intracellular domain of immune effector cell to exhibit a specific immune activity targeting the cell expressing the antigen of interest (e.g., MSLN). In the present invention, the expression “CAR-expressing immune effector cell” refers to an immune effector cell that expresses CAR and have an antigen specificity determined by the targeting domain of the CAR. Methods for preparing CARs (e.g., for cancer treatment) are known in the art; see, for example, Park et al, Trends Biotechnol., 29:550-557, 2011; Grupp et al, N Engl J Med., 368:1509-1518, 2013; Han et al., J. Hematol. Oncol., 6:47, 2013; PCT Patent Publications WO2012/079000, WO2013/059593; and U.S. Patent Publication 2012/0213783, all of which are incorporated herein by reference in their entirety.

As used herein, the terms “extracellular antigen-binding domain” refers to a polypeptide capable of specifically binding to the antigen or receptor of interest. This domain will be able to interact with cell surface molecules. For example, extracellular antigen-binding domains can be selected to recognize antigens that are used as cell surface markers of target cells associated with a particular disease state.

As used herein, the term “intracellular signaling domain” refers to a protein portion that transmits effector signal/function signal and directs a cell to perform a specialized function. Thus, the intracellular signaling domain has the ability to activate at least one normal effector function of CAR-expressing immune effector cells. For example, the effector function of T cells can be cytolytic activity or helper activity, including secretion of cytokines.

As used herein, the term “primary signaling domain” refers to a protein portion that is capable of modulating primary activation of the TCR complex in a stimulatory manner or in an inhibitory manner. Primary signaling domains that act in a stimulatory manner typically contain a signaling motif known as immunoreceptor tyrosine-based activation motif (ITAM). Non-limiting examples of ITAM containing primary signaling domain particularly useful in the present invention include those derived from TCRζ, FcRγ, FcRβ, CD3γ, CD3δ, CD3ε, CD3ζ, CD22, CD79a, CD79b, and CD66d.

As used herein, the term “costimulatory signaling domain” refers to an intracellular signaling domain of a costimulatory molecule. The costimulatory molecule is a cell surface molecule other than antigen receptor or Fc receptor, which, upon binding to an antigen, provides a secondary signal required for efficient activation and function of T lymphocyte. Non-limiting examples of such costimulatory molecule include CARD11, CD2, CD7, CD27, CD28, CD30, CD40, CD54 (ICAM), CD83, CD134 (OX40), CD137 (4-1BB), CD150 (SLAMF1), CD270 (HVEM), CD278 (ICOS), DAP10.

As used herein, the term “pharmaceutically acceptable carrier and/or excipient” refers to a carrier and/or excipient that is pharmacologically and/or physiologically compatible with the subject and the active ingredient, which is well known in the art (see, for example, Remington's Pharmaceutical Sciences. Edited by Gennaro AR. 19th ed. Pennsylvania: Mack Publishing Company, 1995), and includes, but is not limited to: sterile water, physiological saline, pH adjusting agent, surfactant, adjuvant, ionic strength enhancer, diluent, agent for maintaining osmotic pressure, agent for delaying absorption, preservative. For example, the pH adjusting agent includes, but is not limited to, phosphate buffer. The surfactant includes, but is not limited to, cationic, anionic or nonionic surfactant, such as Tween-80. The ionic strength enhancer includes, but is not limited to, sodium chloride. The preservative includes, but is not limited to, various antibacterial and antifungal agents, such as p-hydroxy-benzoate ester, chlorobutanol, phenol, sorbic acid, etc. The agent for maintaining osmotic pressure includes, but is not limited to, sugar, NaCl, and their analogues. The agent for delaying absorption includes, but is not limited to, monostearate salt and gelatin. The diluent includes, but is not limited to, water, aqueous buffer (e.g., buffered saline), alcohol and polyol (e.g., glycerol), etc. The preservative includes, but is not limited to, various antibacterial and antifungal agents such as thimerosal, 2-phenoxyethanol, p-hydroxy-benzoate ester, chlorobutanol, phenol, sorbic acid, etc. The term “stabilizer” has the meaning commonly understood by those skilled in the art, and it can stabilize a desired activity of an active ingredient in a drug, includes but is not limited to sodium glutamate, gelatin, SPGA, sugar (e.g., sorbitol, mannitol, starch, sucrose, lactose, glucan, or glucose), amino acid (e.g., glutamic acid, glycine), protein (e.g., dry whey, albumin or casein) or degradation product thereof (e.g., lactalbumin hydrolyzate), etc. In certain exemplary embodiments, the pharmaceutically acceptable carrier or excipient comprises a sterile injectable liquid (e.g., aqueous or non-aqueous suspension or solution). In certain exemplary embodiments, the sterile injectable liquid is selected from the group consisting of water for injection (WFI), bacteriostatic water for injection (BWFI), sodium chloride solution (e.g., 0.9% (w/v) NaCl), dextrose solution (e.g., 5% dextrose), surfactant-containing solution (e.g., 0.01% polysorbate-20), pH buffered solution (e.g., phosphate-buffered solution). Ringer's solution, and any combination thereof.

As used herein, the term “prevention” refers to a method that is performed to prevent or delay the occurrence of a disease or disorder or symptom (e.g., tumor) in a subject. As used herein, the term “treatment” refers to a method that is performed to obtain a beneficial or desired clinical outcome. For the purposes of the present invention, the beneficial or desired clinical outcome includes, but is not limited to, alleviation of symptom, reduction in the extent of a disease, stabilization (i.e., not worsening) of a disease state, delaying or slowing the progression of a disease, amelioration or remission of a disease state, and relief of symptom (whether in part or in whole), whether detectable or undetectable. In addition, “treatment” can also refer to prolonging survival as compared to an expected survival (if not receiving treatment).

As used herein, the term “subject” refers to a mammal, such as a primate, such as a human. In certain embodiments, the term “subject” refers to a living organism in which an immune response can be elicited. In certain embodiments, the subject (e.g., human) has, or is at risk for, tumor (e.g., tumor associated with MSLN).

As used herein, the term “effective amount” refers to an amount sufficient to obtain, or at least partially obtain, the desired effect. For example, a prophylactically effective amount of a disease (e.g., tumor) refers to an amount sufficient to prevent, arrest, or delay the onset of the disease (e.g., tumor): a therapeutically effective amount of a disease refers to an amount sufficient to cure or at least partially prevent the disease and complication thereof in a patient with the disease. Determining such effective amounts is well within the ability of those skilled in the art. For example, an amount effective for therapeutic use will depend on the severity of the disease to be treated, the general state of the patient's own immune system, the patient's general conditions such as age, weight and sex, the mode of administration of drug, and additional therapy applied simultaneously, and so on.

As used herein, the term “immune cell” refers to a cell involved in an immune response, for example, involved in promoting immune effector function. Examples of immune cell include T cell (e.g., α/β T cells and γ/δ T cells), B cell, natural killer (NK) cell, natural killer T (NKT) cell, mast cell, and bone marrow-derived macrophage.

The immune cell of the present invention can be self/autologous (“self”) or non-self (“non-autologous”, such as allogeneic, syngeneic or xenogeneic). As used herein, “self” refers to cells from the same subject: “allogeneic” refers to cells of the same species that are genetically different from the cell for comparison: “syngeneic” refers to cells from different subjects that genetically same with the cell for comparison: “xenogeneic” refers to cells from species different from the cell for comparison. In a preferred embodiment, the cells of the present invention are allogeneic.

Exemplary immune cells that can be used in the CAR described herein include T lymphocyte and/or NK cell. The term “T cell” or “T lymphocyte” is well known in the art and is intended to include thymocyte, immature T lymphocyte, mature T lymphocyte, resting T lymphocyte or activated T lymphocyte. The T cell may be T helper (Th) cell, such as T helper 1 (Th1) or T helper 2 (Th2) cell. The T cell can be helper T cell (HTL: CD4 T cell) CD4 T cell, cytotoxic T cell (CTL; CD8 T cell), CD4CD8 T cell, CD4CD8 T cell or any other subset of T cell. In certain embodiments, the T cell can include naive T cell and memory T cell.

Those of skill in the art will appreciate that other cells can also be used as immune cells with the CAR as described herein. Specifically, the immune cells also include NK cell, monocyte, macrophage or dendritic cell, NKT cell, neutrophil, and macrophage. The immune cells also include progenitor cells of immune cells, wherein the progenitor cells can be induced in vivo or in vitro to differentiate into immune cells. Thus, in certain embodiments, the immune cells include progenitor cells of immune cells, such as hematopoietic stem cells (HSCs) within a population of CD34+ cells derived from umbilical cord blood, bone marrow, or peripheral blood, which will be differentiated into mature immune cells in a subject after administration, or which can be induced in vitro to differentiate into mature immune cells.

As used herein, the term “engineered immune cell” refers to an immune cell capable of expressing any one of the CARs described herein, or introduced therein any one of the isolated nucleic acid or vector as described herein. The CAR polypeptide can be synthesized in situ in the cell after the polynucleotide encoding the CAR polypeptide has been introduced into the cell by a variety of methods. Alternatively, the CAR polypeptide can be produced extracellularly and then introduced into the cell. Methods of introducing polynucleotide constructs into cells are known in the art. In some embodiments, stable transformation methods can be used to integrate the polynucleotide construct into the genome of the cell. In other embodiments, transient transformation methods can be used to transiently express the polynucleotide construct, and the polynucleotide construct is not integrated into the genome of the cell. In other embodiments, virus-mediated methods can be used. Polynucleotides can be introduced into cells by any suitable method, such as recombinant viral vectors (e.g., retroviruses, adenoviruses), liposomes, and the like. Transient transformation methods include, for example, but are not limited to, microinjection, electroporation, or particle bombardment. The polynucleotide can be contained in a vector, such as a plasmid vector or a viral vector.

As used herein, the term “immune effector function” refers to the function or response of an immune effector cell that enhances or facilitates an immune attack on a target cell (e.g., kills the target cell, or inhibits its growth or proliferation). For example, the effector function of T cell can be cytolytic activity or helper activity, including secretion of cytokine.

### Beneficial Effects of the Present Invention

Chimeric antigen receptor (CAR)-T cell therapy is considered to be one of the most promising cancer treatments compared to traditional tumor treatments such as surgery, radiotherapy, and chemotherapy. However, due to the complex tumor microenvironment of solid tumors that greatly limits the therapeutic effect of CAR-T cell therapy in solid tumors, how to enhance the efficacy of CAR-T cell therapy in solid tumors is the focus of current research.

The present invention provides a chimeric antigen receptor that targets MSLN comprising the antibody or antigen-binding fragment thereof of the present invention, and the immune effector cell expressing the chimeric antigen receptor of the present invention has an enhanced effector function (e.g., tumor-killing activity and cytokine-releasing activity) as compared to the known MSLN-targeting CAR-T. In addition, in the present invention, co-expression of the CAR of the present invention and PD-1 antibody can block the binding between PD-1 and PD-L1 to restore the activity of T cells, thereby enhancing the immune response; and co-expression with mIL-15 can promote the proliferation and activation of T and NK cells, thereby enhancing the tumor-killing effect of CAR-T cells.

The embodiments of the present invention will be described in detail below with reference to the drawings and examples, but those skilled in the art will understand that the following drawings and examples are only used to illustrate the present invention, rather than limit the scope of the present invention. The various objects and advantageous aspects of the present invention will become apparent to those skilled in the art from the accompanying drawings and the following detailed description of the preferred embodiments.

### BRIEF DESCRIPTION OF THE DRAWINGS

FIG. 1 shows the structure of the chimeric antigen receptor constructed in Example 2, in which the anti-MSLN binding domain is scFv, and selected from G5, G9 and G16 respectively.

FIG. 2 shows the detection results of the killing activity of G5-CAR-T, G9-CAR-T and G16-CAR-T on NCI-H226 target cells.

FIG. 3 shows the detection results of secretion levels of IL2, TNF-α and IFN-γ of G16-CAR-T cells after activation by NCI-H226 target cells.

FIG. 4 shows the detection results of the killing activity of G5-CAR-T, G9-CAR-T and G16-CAR-T on SKOV-3 target cells.

FIG. 5 shows the detection results of secretion levels of IL2, TNF-α and IFN-γ of G5-CAR-T, G9-CAR-T and G16-CAR-T cells after activation by SKOV-3 target cells.

FIG. 6 shows the detection results of the killing activity of G16-CAR-T, G16-PD1-CAR-T, G16-mIL15-CAR-T and G16-PD1-mIL15-CAR-T on SKOV-3 target cells.

FIG. 7 shows the detection results of the killing activity of G16-CAR-T on negative A431 cells.

FIG. 8 shows the detection results of secretion levels of IL2, TNF-α and IFN-γ of G16-CAR-T after treatment with negative A431 cells.

FIG. 9 shows the in vivo killing ability of G16-CAR-T on PANC1 target cells in mice.

FIG. 10 shows the in vivo killing ability of G16-CAR-T. G16-PD1-CAR-T and G16-PD1-mIL15-CAR-T on SKOV-3 target cells in mice.

### SEQUENCE INFORMATION

The sequence information involved in the present invention is provided as follows:

| SEQ | |
|---|---|
| ID NO | Description |
| 1 | G16 VH |
| 2 | G16 VL |
| 3 | Kabat |
| | G16 CDR-H1 |
| 4 | Kabat |
| | G16 CDR-H2 |
| 5 | Kabat/Chothia |
| | G16 CDR-H3 |
| 6 | Kabat/Chothia |
| | G16 CDR-L1 |
| 7 | Kabat/Chothia |
| | G16 CDR-L2 |
| 8 | Kabat/IMGT/ |
| | Chothia |
| | G16 CDR-L3 |
| 9 | IMGT |
| | G16 CDR-H1 |
| 10 | IMGT |
| | G16 CDR-H2 |
| 11 | IMGT |
| | G16 CDR-H3 |
| 12 | IMGT |
| | G16 CDR-L1 |
| 13 | IMGT |
| | G16 CDR-L2 |
| 14 | Chothia |
| | G16 CDR-H1 |
| 15 | Chothia |
| | G16 CDR-H2 |
| 16 | G5 VH |
| 17 | G5 VL |
| 18 | Kabat |
| | G5 CDR-H1 |
| 19 | Kabat |
| | G5 CDR-H2 |
| 20 | Kabat/Chothia |
| | G5 CDR-H3 |
| 21 | Kabat/Chothia |
| | G5 CDR-L1 |
| 22 | Kabat/Chothia |
| | G5 CDR-L2 |
| 23 | Kabat/IMGT/ |
| | Chothia |
| | G5 CDR-L3 |
| 24 | IMGT |
| | G5 CDR-H1 |
| 25 | IMGT |
| | G5 CDR-H2 |
| 26 | IMGT |
| | G5 CDR-H3 |
| 27 | IMGT |
| | G5 CDR-L1 |
| 28 | IMGT |
| | G5 CDR-L2 |
| 29 | Chothia |
| | G5 CDR-H1 |
| 30 | Chothia |
| | G5 CDR-H2 |
| 31 | G9 VH |
| 32 | G9 VL |
| 33 | Kabat |
| | G9 CDR-H1 |
| 34 | Kabat |
| | G9 CDR-H2 |
| 35 | Kabat/Chothia |
| | G9 CDR-H3 |
| 36 | Kabat/Chothia |
| | G9 CDR-L1 |
| 37 | Kabat/Chothia |
| | G9 CDR-L2 |
| 38 | Kabat/IMGT/Chothia |
| | G9 CDR-L3 |
| 39 | IMGT |
| | G9 CDR-H1 |
| 40 | IMGT |
| | G9 CDR-H2 |
| 41 | IMGT |
| | G9 CDR-H3 |
| 42 | IMGT |
| | G9 CDR-L1 |
| 43 | IMGT |
| | G9 CDR-L2 |
| 44 | Chothia |
| | G9 CDR-H1 |
| 45 | Chothia |
| | G9 CDR-H2 |
| 46 | nucleotide sequence of |
| | G16 VH |
| 47 | nucleotide sequence of |
| | G16 VL |
| 48 | nucleotide sequence of G5 |
| | VH |
| 49 | nucleotide sequence of G5 |
| | VL |
| 50 | nucleotide sequence of G9 |
| | VH |
| 51 | nucleotide sequence of G9 |
| | VL |
| 52 | amino acid sequence of |
| | Linker |
| 53 | nucleotide sequence of |
| | Linker |
| 54 | amino acid sequence of |
| | G16-scFv |
| 55 | nucleotide sequence of |
| | G16-scFv |
| 56 | amino acid sequence of |
| | G5-scFv |
| 57 | nucleotide sequence of G5- |
| | scFv |
| 58 | amino acid sequence of |
| | G9-scFv |
| 59 | nucleotide sequence of G9- |
| | scFv |
| 60 | amino acid sequence of |
| | Signal peptide 1 |
| 61 | nucleotide sequence of |
| | Signal peptide 1 |
| 62 | amino acid sequence of |
| | CD8 hinge region |
| 63 | nucleotide sequence of |
| | CD8 hinge region |
| 64 | amino acid sequence of |
| | CD8 transmembrane region |
| 65 | nucleotide sequence of CD8 |
| | transmembrane region |
| 66 | amino acid sequence of |
| | Intracellular signaling |
| | domain (4-1BB) |
| 67 | nucleotide sequence of |
| | Intracellular signaling |
| | domain (4-1BB) |
| 68 | amino acid sequence of |
| | Intracellular signaling |
| | domain (CD3ζ) |
| 69 | nucleotide sequence of |
| | Intracellular signaling |
| | domain (CD3ζ) |
| 70 | amino acid sequence of |
| | Intracellular signaling |
| | domain (4-1BB-CD3ζ) |
| 71 | nucleotide sequence of |
| | Intracellular signaling |
| | domain (4-1BB-CD3ζ) |
| 72 | amino acid sequence of P2A |
| 73 | nucleotide sequence-1 of |
| | P2A |
| 74 | amino acid sequence of |
| | Signal peptide 2 |
| 75 | nucleotide sequence of |
| | Signal peptide 2 |
| 76 | nucleotide sequence-2 of |
| | P2A |
| 77 | amino acid sequence of PD1- |
| | scFv |
| 78 | nucleotide sequence of PD1- |
| | scFv |
| 79 | PD1-scFv VH |
| 80 | PD1-scFv VL |
| 81 | amino acid sequence of |
| | mIL 15 |
| 82 | nucleotide sequence of |
| | mIL 15 |
| 83 | amino acid sequence of G16- |
| | CAR |
| 84 | nucleotide sequence of G16 |
| | CAR |
| 85 | nucleotide sequence of G16- |
| | PD1 CAR |
| 86 | nucleotide sequence of G16- |
| | mIL15 CAR |
| 87 | nucleotide sequence of G16- |
| | PD1-mIL 15 CAR |
| 88 | amino acid sequence of G5- |
| | CAR |
| 89 | nucleotide sequence of G5 |
| | CAR |
| 90 | amino acid sequence of G9- |
| | CAR |
| 91 | nucleotide sequence of G9 |
| | CAR |
| 92 | Primer PKLT1F |
| 93 | Primer PKLT1R |
| 94 | amino acid sequence of Fc |
| | protein |

### EXAMPLES

The present invention will now be described with reference to the following examples, which are intended to illustrate, but not limit, the present invention.

Unless otherwise specified, the molecular biology experimental methods and immunoassays used in the present invention were performed basically by referring to those described in J. Sambrook et al., Molecular Cloning: A Laboratory Manual, 2nd Edition, Cold Spring Harbor Laboratory Press, 1989, and F. M. Ausubel et al., Refined Molecular Biology Laboratory Manual, 3rd Edition, John Wiley & Sons, Inc., 1995. Those skilled in the art appreciate that the examples describe the present invention by way of example and are not intended to limit the scope sought to be protected by the present invention.

### Example 1: Preparation of Single Chain Antibody (scFv) Specific for Human MSLN

### 1) Phage Library Screening for MSLN-scfv

The peripheral blood of healthy people was collected, PBMC cells were isolated, total RNA was extracted from the cells, and cDNA was obtained by reverse transcription. Phage antibody libraries were constructed using cDNA and primers for amplifying heavy chain variable region and light chain variable region of anti-MSLN antibody.

Magnetic beads were washed with 0.05% PBST; 1.33 ml of fully human phage library was mixed with 50 μl of the magnetic beads, the mixture was gently rolled for 10 minutes, the magnetic beads were separated with a magnetic separator, and the supernatant was transferred to new magnetic beads, the magnetic beads were separated after 30 minutes of rolling. The same steps were performed for the third time, except with a rolling time of 60 minutes. The supernatant was pipetted and transferred into a new centrifuge tube, added with 10 μg of biotinylated MSLN and magnetic beads, subjected to binding for 30 minutes, and the centrifuge tube was placed on a magnetic stand for 2 minutes. The supernatant was transferred to a 15 ml centrifuge tube and stored at 4° C. The magnetic beads were washed 14 times, mPBST for the first 4 times, and PBST for the rest times, with 1 ml of washing solution for each time. The phage bound on the magnetic beads was finally eluted with 1 ml of 100 mM triethylamine, followed by neutralization of the eluate with 100 ul of 1M Tris-HCl (pH 7.5). 10 ul of phage was taken, and diluted with 90 ul of PBS. 10 ul of the dilution was taken and added with 90 ul of SS320 cells (OD600=0.5-0.6) to amplify the phage, and the titer was measured. The amplified phage was transferred to a 50 ml centrifuge tube and centrifuged at 9000 g, 4° C., for 15 min. The supernatant was transferred to a new centrifuge tube, added with 20% PEG6000/2.5M NaCl, mixed well and placed on ice for 2 h. After centrifugation at 12,000 g for 30 minutes, the supernatant was discarded, the phage pellets were resuspended with 0.5 ml of PBST: after centrifuged at 10,000 g for 8 minutes, the supernatant was transferred to a new test tube, a part thereof was taken for titer determination, and the rest was subject to the second and third rounds of screening.

### 2) ELISA Detection of Monoclonal Phage

Production of monoclonal phage in microtiter plate: A single colony was inoculated into a 96-deep-well plate, with each well containing 300 ul of 2YT medium, 10 μg/ml Tet, and incubated at 37° C., for 5-6 hours at 250 rpm. 150 ul of culture was taken out of each well, added with an equal volume of sterile 50% glycerol, and stored at −80° C. 25 ul of helper phage (2.5×10⁹) was added to each well and incubated at 37° C., for 30 minutes. After supplementing with 150 ul of 2YT medium and 200 μg/ml Amp, 20 μg/ml Tet, 100 μg/ml kanamycin, 2 mM IPTG, culturing was performed at 32° C., 250 rpm overnight. On the second day, the deep-well plate was centrifuged at 3200 g for 15 minutes, and the supernatant was transferred to a new 96-well plate and stored at 4° C.

ELISA detection: Biotin-labeled MSLN (MSN-H826x, ACROBiosystems) was diluted with PBS to 100 μg/ml-8 ug/ml, coated in streptavidin 96-well plate, and allowed to stand overnight at 4° C. The wells were washed once with 300 ul of PBS. 200 ul of mPBST (2% milk) was used to perform blocking at 37° C., for 1 hour. The blocking solution was discarded, 80 ul of phage supernatant and 80 ul of mPBST were added to each well, and allowed to stand at room temperature for 1 hour. The plate was washed 5 times with PBST, added with 100 μl of anti-M13-HRP (diluted to 0.4 ug/ml in mPBST), and allowed to stand at room temperature for 1 hour. The plate was washed 5 times with PBST, added with 100 μl of TMB, reacted at room temperature for 3 min, and then added with 100 ul of stop solution (0.2M H₂SO₄). Optical density at 450 nm was detected using an enzyme-linked immunometric meter.

MSLN scFv sequencing: 158 single clones were selected for sequencing according to the ELISA detection results, and the scfv sequences were obtained. The forward and reverse primers used for sequencing were: PKLT1F (SEQ ID NO: 92): PKLT1R (SEQ ID NO: 93). The sequence results were analyzed using Seqcher software, and three candidate scfvs were finally obtained, named: G16, G5 and G9.

TABLE 1
Variable regions and CDR sequences of scFvs

| | | G16 | G5 | G9 |
|---|---|---|---|---|
| scFv | | (SEQ ID NO:) | (SEQ ID NO:) | (SEQ ID NO:) |
| VH | | 1 | 16 | 31 |
| VL | | 2 | 17 | 32 |
| Kabat | CDR-H1 | 3 | 18 | 33 |
| | CDR-H2 | 4 | 19 | 34 |
| | CDR-H3 | 5 | 20 | 35 |
| | CDR-L1 | 6 | 21 | 36 |
| | CDR-L2 | 7 | 22 | 37 |
| | CDR-L3 | 8 | 23 | 38 |
| IMGT | CDR-H1 | 9 | 24 | 39 |
| | CDR-H2 | 10 | 25 | 40 |
| | CDR-H3 | 11 | 26 | 41 |
| | CDR-L1 | 12 | 27 | 42 |
| | CDR-L2 | 13 | 28 | 43 |
| | CDR-L3 | 8 | 23 | 38 |
| Chothia | CDR-H1 | 14 | 29 | 44 |
| | CDR-H2 | 15 | 30 | 45 |
| | CDR-H3 | 5 | 20 | 35 |
| | CDR-L1 | 6 | 21 | 36 |
| | CDR-L2 | 7 | 22 | 37 |
| | CDR-L3 | 8 | 23 | 38 |

3) Construction of scFv-Fc and Analysis of Antibody Aggregate

The three candidate scFv sequences were linked with Fc (human IgG1) sequence and constructed in TGEX-KAL vector, and then transfected into expi293 cells for expression and purification of scFv-Fc protein. The Fc (human IgG1) sequence was shown in SEQ ID NO: 94. The experimental results of SEC analysis showed that the area of the monomer peaks (main peaks) of the three candidate sequences accounted for more than 85%.

TABLE 2
SEC data of candidate scFv-Fc proteins

| | Main peak | | | |
|---|---|---|---|---|
| Sample | RT (min) | % Area | HMW (%) 1 | LMW(%) 2 |
| G5 scFv-Fc | 4.48 | 95.4 | 4.8 | ND |
| G9 scFv-Fc | 4.76 | 87.3 | 12.7 | ND |
| G16 scFv-Fc | 4.33 | 88.3 | 11.7 | ND |

Note:
ND means not detected.

### 4) MSLN Cell Binding Assay

In order to identify the binding affinity of MSLN scFv-Fc protein, the MSLN scFv-Fc protein was serially diluted and used to stain the three selected cell lines expressing MSLN, and the cell binding ability was detected by flow cytometry. The results were shown in the table below. The EC50 of anti-MSLN antibodies G9 and G16 to three kinds of MSLN-positive cells were 2-23 nM, showing good binding affinity to MSLN.

TABLE 3
Affinity determination results of candidate
scFv-Fc to 3 kinds of MSLN-positive cells

| ID | Cell | G9 scFv-Fc | G16 scFv-Fc |
|---|---|---|---|
| EC50 | 293T/MSLN+ | 17.69 | 1.576 |
| (nM) | OVCAR-3 | 22.3 | 4.92 |
| | NCI-H596 | 18.7 | 2.25 |

### Example 2: Construction of Lentiviral Plasmid and Viral Packaging

### 2.1 Construction of Lentiviral Plasmid:

### (1) Connection Order of Each Part of Chimeric Antigen Receptor

First, based on the scFv sequence in the above example, a CAR was further constructed. The intracellular domain of CD137 and the ITAM region of CD3Zeta were used as activation signals, fused with the above scFv, as well as signal peptide, CD8 hinge region, and CD8 transmembrane region to construct the expression vector of a chimeric antigen receptor. The constructed chimeric antigen receptor had structure as shown in the table below.

TABLE 4
Structure of basic chimeric antigen receptor

| | N-terminal→C-terminal, amino acid sequence of each | | | | | | |
|---|---|---|---|---|---|---|---|
| | element (SEQ ID NO:) | | | | | | |
| | | | | | Intracellular | Full- | Encoding |
| CAR | Signal | | Hinge | Transmembrane | signaling | length | nucleotide |
| name | peptide | scFv | region | region | domain | sequence | sequence |
| G16-CAR | 60 | 54 | 62 | 64 | 70 | 83 | 84 |
| G5-CAR | 60 | 56 | 62 | 64 | 70 | 88 | 89 |
| G9-CAR | 60 | 58 | 62 | 64 | 70 | 90 | 91 |

On the basis of the above basic CAR structure, the sequence encoding P2A self-cleaving peptide was used to link the sequence encoding one or more additional biologically active molecules (e.g., PD-1 scFv and/or mIL-15, in which the sequences encoding multiple biologically active molecules could be further linked by nucleotide sequence of P2A self-cleaving peptide) to obtain a co-expression CAR. When the above co-expression CAR was expressed in cells, the biologically active molecules linked to the P2A sequence were secreted outside the CAR-T cells or expressed to anchor on the CAR-T cell membrane to exert a synergistic anti-tumor effect. For example, since MSLN CAR-T specifically binds to tumor cells, G16-PD1-CAR-T can relieve or eliminate immunosuppression by secreting anti-PD-1 antibody after activation, thereby increasing an anti-tumor effect: after G16-mIL15-CAR-T is activated, CAR-T cells express membrane-bound IL-15, and the membrane-bound IL-15 stimulates the secretion of some cytokines, thereby enhancing an anti-tumor effect and prolonging the anti-tumor effect. The structure of the nucleic acid sequence encoding the co-expression CAR was shown in the table below.

TABLE 5
Structure of nucleic acid sequence encoding Co-expression CAR

| | 5′→3′ (SEQ ID NO:) | | | | | | |
|---|---|---|---|---|---|---|---|
| | | | | | | | Full- |
| | Basic | | Signal | PD-1 | | | length |
| CAR name | CAR | P2A | peptide-2 | scFv | P2A | mIL15 | sequence |
| G16-PD1 CAR | 84 | 73 | 75 | 78 | None | None | 85 |
| G16-mIL15 CAR | 84 | 76 | None | None | None | 82 | 86 |
| G16-PD1-mIL15 CAR | 84 | 73 | 75 | 78 | 76 | 82 | 87 |

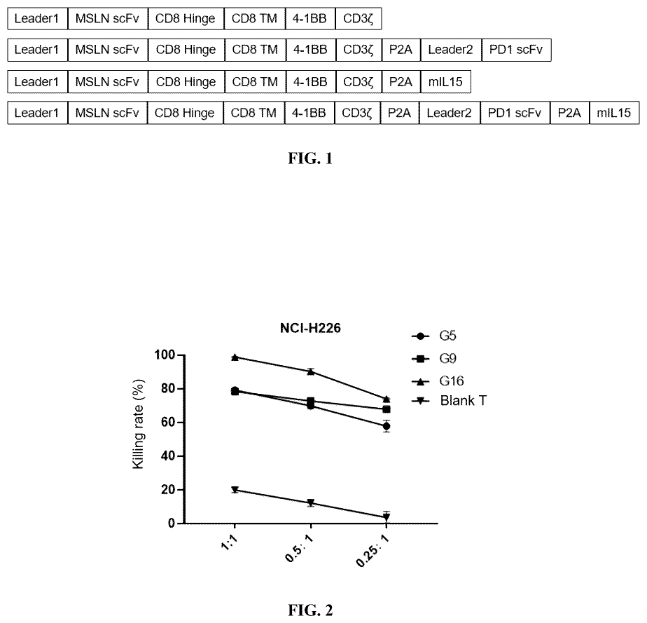

The structures of the basic CAR and co-expression CAR as constructed above can be seen in FIG. 1.

(2) The nucleotide sequences after codon optimization of the scFv and mIL15 in the above structures was synthesized and constructed into Lenti-4-EF1a vector outsource. Single clones were picked for cultivation and seed preservation. Finally, the plasmids were extracted and sequenced. The bacterial solutions of with correct sequencing results were used to prepare lentiviral plasmids.

### 2.2 Virus Packaging:

A mixture of the above-constructed CAR lentiviral plasmids and transfection reagent was added dropwise to 293T (ATCC) cells, and mixed well by gently shaking the culture dish. The culture dish was placed in a 37° C., 5% CO₂ incubator; after 6-8 hours of incubation, the medium containing the transfection reagent was discarded, and replaced with fresh complete medium. The virus-containing medium supernatants after 48 hours and 72 hours of continuous culture were collected, added with PEG and allowed to precipitate overnight at 4° C., and then centrifuged at 4000×g for 1 hour at 4° C. After centrifugation, in a biological safety cabinet, the liquid in the centrifuge tube was carefully pipetted out, 300 μL of virus freezing solution was added to resuspend the pellet, and the virus was stored at −80° C.

### Example 3: Preparation of CAR-T Cells

### 1) Isolation of Primary T Cells:

(1) Human PBMC cells were isolated with lymphocyte separation medium (GE), placed in a 37° C., 5% CO₂ incubator, added with 100 μl/mL CD3 antibody and CD28 antibody, mixed well, and incubated at room temperature for 15 minutes.
(2) Magnetic beads were taken, pipetted up and down at least 5 times with a pipette, and mixed thoroughly.
(3) The magnetic beads were pipetted and added to the above sample, 50 μl per mL, mixed well, and incubated at room temperature for 10 minutes.
(4) Complete medium was added to reach a total volume of 2.5 mL in the tube, the tube (uncapped) was inserted into a magnetic pole, and allowed to stand at room temperature for 5 minutes.
(5) After the incubation, the tube continued to stay in the magnetic pole and was gently inverted to pour out the cells in the tube.
(6) The cells were resuspended in X-vivo 15 medium, and added with 10% FBS, 300 U/mL IL-2, 5 ng/mL IL-15 and 10 ng/mL IL-7.

### 2) Activation of T Cells:

The cell density was adjusted to 1×10⁶ cells/mL, added with cytokine/antibody complex (with final concentrations of 300 U/mL IL-2, 10 ng/mL IL-7, 5 ng/ml IL-15, 500 ng/ml Anti-CD3 (OKT3), 2 μg/mL Anti-CD28), and cultured continuously for 48 hours.

### 3) Virus Infection:

(1) According to MOI=20, the required amount of virus was calculated. The calculation formula was as follows: the required amount of virus (mL)=(MOI*number of cells)/virus titer
(2) After the virus was taken out of the −80° C., refrigerator, it was quickly thawed in a 37° C. water bath. The virus in the amount calculated above was added to a 6-well plate, added with DEAE with a final concentration of 5 μg/mL, mixed well, the 6-well plate were sealed at four sides with parafilm, and centrifuged at 800×g for 1 hour.
(3) After centrifugation, the parafilm was torn off, and the 6-well plate was placed in a 37° C. 5% CO₂ incubator, and continuously cultured for 24 hours.
(4) After being centrifuged at 250×g for 10 minutes, the medium supernatant containing the virus was discarded, the cell pellet was resuspended with fresh medium, the cells were transferred to a new 6-well plate, and continuously cultured for 3-6 days for later use.

### Example 4: Detection of Positive Rate of CAR-T Cells

The nucleic acid sequence encoding the CAR was expressed under the drive of promoter, and the lentivirus-transfected T cells were labeled with biotin-labeled MSLN antigen, and then detected with fluorescently labeled streptavidin, and determined by flow cytometry, which reflected the expression level of the CAR on the surface of T cells. The CAR positive rate of the CAR-T cells obtained in Example 3 was detected by the above method, and the FACS detection results were shown in the following table. The results showed that the CAR positive rates of all CAR-T cells were greater than 20%, indicating that after effector cells were transfected with lentivirus, CAR was successfully expressed, and MSLN-CAR T cells were successfully constructed.

TABLE 6
Detection results of positive rate of CAR

| Chimeric antigen receptor | CAR positive rate |
|---|---|
| G5 | 50.45 |
| G9 | 38.04 |
| G16 | 41.47 |

### Example 5: Evaluation of Killing Activity of CAR-T on NCI-H226 Target Cells

### 5.1 Evaluation of Lysis Ability of CAR-T on NCI-H226 Target Cells

Luciferase gene was integrated into the genome of NCI-H226 cells by lentiviral transduction to obtain NCI-H226 human lung squamous carcinoma cells that could stably express Luciferase (NCI-H226-luc). NCI-H226-luc cells were digested with 0.25% trypsin, and the digestion was terminated by 1640 medium containing 10% FBS. After centrifugation, the cells were resuspended, the cell density was adjusted to 1×10⁵ cells/mL, and the target cells NCI-H226-luc were inoculated into a 96-well plate at an amount 100 μL/well, and allowed to stand in a 5% CO₂, 37° C., incubator for 30 minutes. G5-CAR-T, G9-CAR-T and G16-CAR-T were collected separately by centrifugation and resuspended in 1640 medium with 10% FBS. G5-CAR-T, G9-CAR-T and G16-CAR-T and blank T cells that were not transfected with CAR were used as effector cells, and then added to the 96-well plate containing NCI-H226-luc at E/T (effector cells/target cells) ratios of 1:1, 0.5:1, 0.25:1, with 100 μL/well: the final volume was made up to 200 μL/well, and incubation was performed in a 5% CO₂, 37° C., incubator for 18 to 24 hours. After the incubation was completed, the plate was taken out of the incubator, added with 20 μl of fluorescence detection reagent, and a microplate reader was used to detect the fluorescence reading.

The detection results of the killing activity of CAR-T were shown in FIG. 2, indicating that all of which could exert the biological activity of lysis on tumor cells. When the ratio of effector cells/target cells was 1, the lysis rate of G16-CAR-T on tumor cells was as high as 98%. When the ratio of effector cells/target cells was 1, the lysis rates of G9-CAR-T and G5-CAR-T to tumor cells reached about 80%.

### 5.2 Cytokine Release Assay

NCI-H226 cells were collected, the cell density was adjusted to 1×10⁵ cells/mL with medium, the target cells were inoculated into a 96-well plate at an amount of 100 μL/well, the G16-CAR-T cells were resuspended with medium: G16-CAR-T and blank T cells that were not transfected with CAR were used as effector cells, and then added to the 96-well plate containing target cells at an E/T (effector cells/target cells) ratio of 1:1, with 100 μL/well: the final volume was made up to 200 μL/well, and incubation was performed overnight in a 37° C., 5% CO₂ incubator. After the incubation, the well plate was taken out of the incubator, centrifuged, and the supernatant was taken for detection of the cytokine release of CAR-T cells by ELISA kits (IL2, TNF-α, IFN-γ).

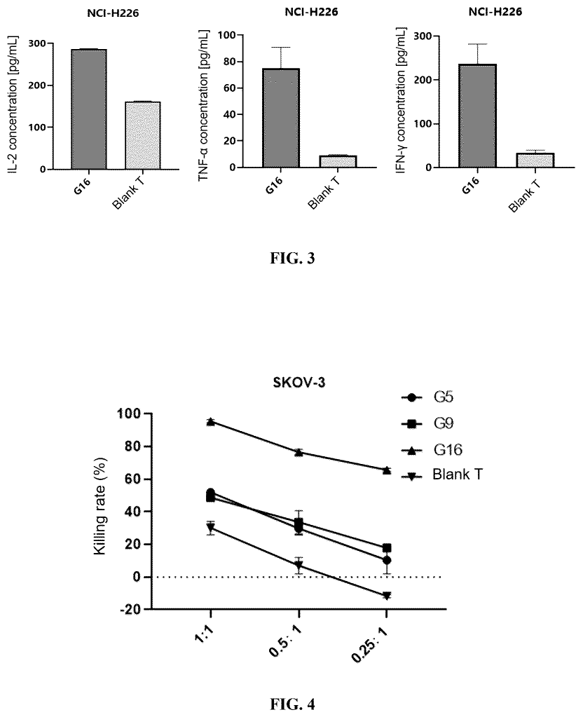

The test results were shown in FIG. 3, which showed that G16-CAR-T could significantly enhance the secretion or release of IL2, TNF-α, and IFN-γ, and had good immune-enhancing activity.

### Example 6: Evaluation of Killing Activity of CAR-T on SKOV-3 Target Cells

### 6.1 Evaluation of Lysis Ability of CAR-T on SKOV-3 Target Cells

Luciferase gene was integrated into the genome of SKOV-3 cells by lentiviral transduction to obtain human ovarian cancer SKOV-3 cells that stably expressed Luciferase (SKOV-3-luc). The SKOV-3-luc cells were digested with 0.25% trypsin, and the digestion was stopped with McCoy′s 5A medium containing 10% FBS. After centrifugation, the cells were resuspended, the cell density was adjusted to 1×10⁵ cells/mL. The target cells SKOV-3-luc were inoculated into a 96-well plate at an amount of 100 μL/mL, and allowed to stand in a 5% CO₂, 37° C., incubator for 30 minutes. G5-CAR-T, G9-CAR-T and G16-CAR-T were collected separately by centrifugation, and resuspended in McCoy′s 5A medium with 10% FBS. The G5-CAR-T, G9-CAR-T, G16-CAR-T and blank T cells that were not transfected with CAR were used as effector cells, and then added into the 96-well plate containing SKOV-3-luc at E/T (effector cells/target cells) ratios of 1:1, 0.5:1 and 0.25:1, with 100 μL/well: the final volume was made up to 200 μL/well, and incubation was performed in a 5% CO₂, 37° C., incubator for 18-24 hours. After the incubation, the well plate was taken out of the incubator, added with 20 μl of fluorescence detection reagent, and a microplate reader was used to detect the fluorescence reading.

The detection results of the killing activity of CAR-T were shown in FIG. 4, indicating that all of which could exert the biological activity of lysis on tumor cells. When the ratio of effector cells/target cells was 1, the lysis rate of G16-CAR-T on tumor cells was up to 95%. When the ratio of effector cells/target cells was 1, the lysis rates of G9-CAR-T and G5-CAR-T on tumor cells were about 50%.

### 6.2 Cytokine Release Assay

SKOV-3-luc cells were collected, the cell density was adjusted to 1×10⁵ cells/mL with medium, target cells were inoculated into a 96-well plate at an amount of 100 μL/well. G5-CAR-T, G9-CAR-T and G16-CAR-T cells were resuspended in medium, the G5-CAR-T, G9-CAR-T, G16-CAR-T and blank T cells that were not transfected with CAR were used as effector cells, and then added to the 96-well plate containing target cells at an E/T (effector cells/target cells) ratio of 1:1, at 100 μL/well: the final volume was made up to 200 μL/well, and incubation was performed overnight in a 5% CO₂, 37° C., incubator. After the incubation, the well plate was taken out of the incubator, centrifuged, and the supernatant was taken for detection of the cytokine release of CAR-T cells by ELISA kits (IL2, TNF-α, IFN-γ).

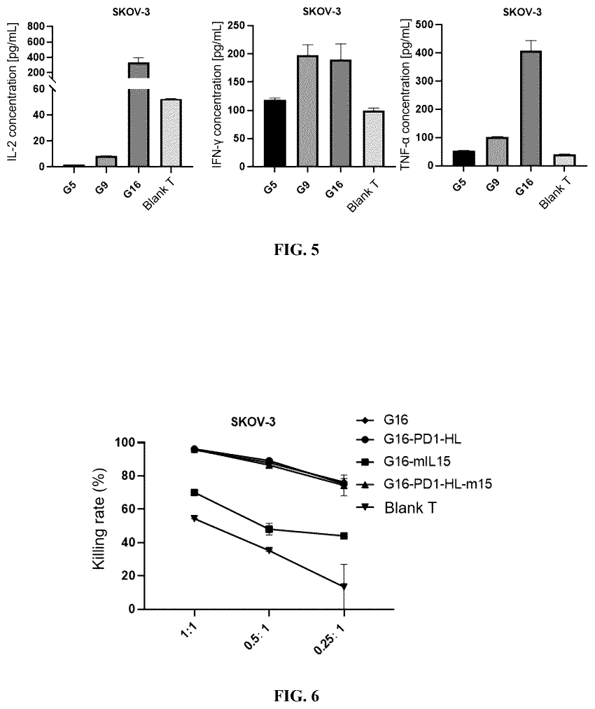

The test results were shown in FIG. 5, which showed that G5-CAR-T, G9-CAR-T and G16-CAR-T could promote the secretion or release of cytokines such as TNF-α and IFN-γ under the stimulation of tumor cells, especially G16-CAR-T could significantly enhance the secretion or release of IL2, TNF-α, and IFN-γ, and had good immune-enhancing activity.

6.3 Evaluation of Killing Activity of CAR-T Co-Expressing PD-1 Antibody and/or mIL-15 on SKOV-3 Target Cells 0.25% trypsin was used to digest SKOV-3-luc cells, and the digestion was stopped with McCoy′s 5A medium containing 10% FBS. After centrifugation, the cells were resuspended, the cell density was adjusted to 1×10⁵ cells/mL: the target cells SKOV-3-luc were inoculated into a 96-well plate at an amount of 100 μL/well, and allowed to stand in a 5% CO₂, 37° C., incubator for 30 minutes. G16-CAR-T, G16-PD1-CAR-T, G16-mIL15-CAR-T, G16-PD1-mIL15-CAR-T were collected by centrifugation and resuspended in McCoy's 5A medium with 10% FBS: the G16-CAR-T, G16-PD1-CAR-T, G16-mIL15-CAR-T, G16-PD1-mIL15-CAR-T, and blank T cells that were not transfected with CAR were used as effector cells, and then added into the 96-well plate containing SKOV-3-luc at E/T (effector cells/target cells) ratios of 1:1, 0.5:1, 0.25:1, at 100 μL/well; and the final volume was made up to 200 μL/well, incubation was performed for 18 to 24 hours in a 37° C., incubator with 5% CO₂. After the incubation, the well plate was taken out of the incubator, added with 20 μl of fluorescence detection reagent, and a microplate reader was used to detect the fluorescence reading.

The detection results of the killing activity of CAR-T were shown in FIG. 6, indicating that all of which could exert the biological activity of lysis on tumor cells. The G16-CAR-T, G16-PD1-CAR-T, G16-PD1-mIL15-CAR-T had comparable biological activity of lysis on tumor cells, and when the ratio of effector cells/target cells was 1, the lysis rate on tumor cells was as high as 98%. When the ratio of effector cells/target cells was 1, the lysis rate of G16-mIL15-CAR-T on tumor cells reached about 73%.

### Example 7: Evaluation of Killing Activity of CAR-T on MSLN-Negative A431 Cells

### 7.1 Evaluation of Lysis Ability of CAR-T on A431 Cells

Luciferase gene was integrated into the genome of MSLN-negative A431 cells by lentiviral transduction to obtain A431 human cutaneous squamous carcinoma cells that stably express Luciferase (A431-luc). A431-luc cells were digested with 0.25% trypsin, and the digestion was terminated in 1640 medium containing 10% FBS. After centrifugation, the cells were resuspended, the cell density was adjusted to 1×10⁵ cells/mL, and the target cells A431-luc were inoculated into a 96-well plate at an amount of 100 μL/well, and allowed to stand in a 5% CO₂, 37° C., incubator for 30 minutes. G16-CAR-T was collected by centrifugation, and resuspended in 1640 medium with 10% FBS. The G16-CAR-T and blank T cells that were not transfected with CAR were used as effector cells, and added into a 96-well plate containing A431-luc at E/T (effector cells/target cells) ratios of 0.5:1, 0.25:1 and 0.125:1, with 100 μL/well: the final volume was made up to 200 μL/well, and incubation was performed in a 37° C., 5% CO₂ incubator for 18-24 hours. After the incubation, the well plate was taken out of the incubator, added with 20 μl of fluorescence detection reagent, and a microplate reader was used to detect the fluorescence reading.

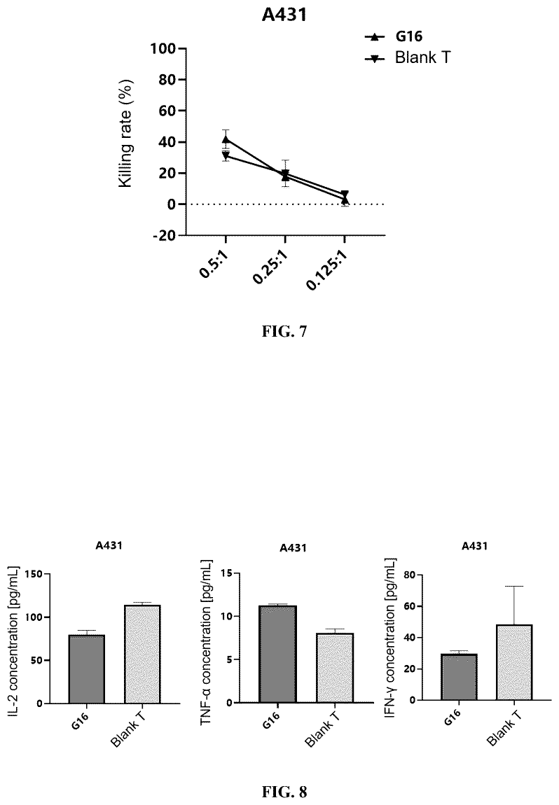

The cytotoxic activity assay results of CAR-T were shown in FIG. 7. The results showed that G16-CAR-T had no cytotoxic activity on MSLN-negative A431 cells, indicating that G16-CAR-T had no off-target killing effect on MSLN-negative cells.

### 7.2 Cytokine Release Assay

A431-luc cells were collected, the cell density was adjusted to 1×10⁵ cells/mL with medium, target cells were inoculated into a 96-well plate at 100 μL/well; and G16-CAR-T and the blank T cells that were not transfected with CAR were resuspended in medium and used as effector cells, and then added to a 96-well plate containing target cells at an E/T (effector cells/target cells) ratio of 1:1, with 100 μL/well; and the final volume was made up to 200 μL/well, and incubation was performed in a 5% CO₂, 37° C., incubator overnight. After the incubation, the well plate was taken out of the incubator, centrifuged, the supernatant was taken for detection of cytokine release of CAR-T cells by ELISA kits (IL2, TNF-α, IFN-γ).

The assay results were shown in FIG. 8. The results showed that for MSLN-negative A431-luc cells, G16-CAR-T did not secrete IL2, TNF-α and IFN-γ, indicating that MSLN-negative cells had no activation effect on G16-CAR-T.

### Example 8: Evaluation of Killing Ability of CAR-T Cells on Target Cells by In Vivo Model

### 8.1 Evaluation of Killing Ability of CAR-T Cells on PANC1 Target Cells by In Vivo Model

Six B-NDG mice were subcutaneously inoculated with 5×10⁶ PANC1 tumor cells on the right side or right scapula. When the average tumor volume reached 100-150 mm³, the mice were randomly divided into two groups, and each mouse was intraperitoneally administrated with cyclophosphamide at 100 mg/kg: 5×10⁶ G16-CAR-T and blank T cells that were not transfected with CAR were reinfused via tail vein on the next day. The tumor diameter was measured with vernier calipers and body weight of mice were measured twice a week. The tumor volume was calculated by the formula: V=0.5a×b², wherein a and b represented the long diameter and short diameter of tumor, respectively.

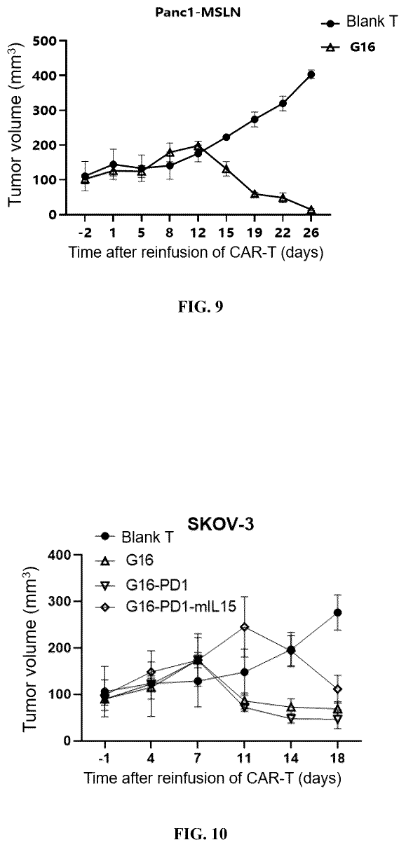

The killing ability of CAR-T cells against target cells was shown in FIG. 9. Compared with the negative control group, G16-CAR-T had a good inhibitory effect on tumor cells. There was no animal death and significant weight loss in all treatment groups during the observation period, and there was no obvious drug toxicity reaction, and the mice tolerated well during the treatment period.

### 8.2 Evaluation of Killing Ability of CAR-T Cells Against SK-OV-3 Target Cells by In Vivo Model

SKOV-3 cells were cultured in monolayer in vitro, and the culture condition was McCoy's 5A medium added with 10% fetal bovine serum, and incubation in a 37° C., incubator with air containing 5% CO₂. Digestion with trypsin-EDTA and passage were performed for 2-3 times per week. The cells in logarithmic growth phase were harvested, counted, for inoculation. Twelve B-NDG mice were subcutaneously inoculated with 1×10⁷ SKOV-3 tumor cells on the right side or right scapula, and when the average tumor volume reached about 100 mm³, the mice were randomly divided into 4 groups, and each mouse was intraperitoneally administered with cyclophosphamide at 100 mg/kg: G16-CAR-T, G16-PD1-CAR-T, G16-PD1-mIL15-CAR-T and blank T cells that were not transfected with CAR were reinfused at 5×10⁶ via tail vein on the next day. The tumor diameter was measured with vernier calipers and body weight of the mice was measured twice per week. The tumor volume was calculated by the formula: V=0.5a× b², wherein a and b represented the long diameter and short diameter of tumor, respectively.

The results of the killing ability of CAR-T cells against target cells were shown in FIG. 10. Compared with the negative control group, the G16-CAR-T, G16-PD1-CAR-T and G16-PD1-mIL15-CAR-T had good inhibitory effects on tumors. By day 18, compared with the negative control group, all treatment groups had significant tumor suppressing effect. There was no animal death and significant weight loss in all treatment groups during the observation period, and there was no obvious drug toxicity reaction, and the mice tolerated well during the treatment period.

Although specific embodiments of the present invention have been described in detail, those skilled in the art will appreciate that various modifications and changes can be made to the details in light of all the teachings that have been published, and that these changes are all within the scope of the present invention. The full division of the present invention is given by the appended claims and any equivalents thereof.

1. An antibody or antigen-binding fragment thereof capable of specifically binding to MSLN, the antibody or antigen-binding fragment thereof comprising:
(1a) the following three heavy chain CDRs defined by the Kabat numbering system: CDR-H1 having the sequence as set forth in SEQ ID NO: 3 or a variant thereof; CDR-H2 having the sequence as set forth in SEQ ID NO: 4 or a variant thereof; CDR-H3 having the sequence as set forth in SEQ ID NO: 5 or a variant thereof; and/or, the following three light chain CDRs defined by the Kabat numbering system: CDR-L1 having the sequence as set forth in SEQ ID NO: 6 or a variant thereof; CDR-L2 having the sequence as set forth in SEQ ID NO: 7 or a variant thereof; CDR-L3 having the sequence as set forth in SEQ ID NO: 8 or a variant thereof;
or
(1b) the following three heavy chain CDRs defined by the IMGT numbering system: CDR-H1 having the sequence as set forth in SEQ ID NO: 9 or a variant thereof; CDR-H2 having the sequence as set forth in SEQ ID NO: 10 or a variant thereof; CDR-H3 having the sequence as set forth in SEQ ID NO: 11 or a variant thereof; and/or, the following three light chain CDRs defined by the IMGT numbering system: CDR-L1 having the sequence as set forth in SEQ ID NO: 12 or a variant thereof; CDR-L2 having the sequence as set forth in SEQ ID NO: 13 or a variant thereof; CDR-L3 having the sequence as set forth in SEQ ID NO: 8 or a variant thereof;
or
(1c) the following three heavy chain CDRs defined by the Chothia numbering system: CDR-H1 having the sequence as set forth in SEQ ID NO: 14 or a variant thereof; CDR-H2 having the sequence as set forth in SEQ ID NO: 15 or a variant thereof; CDR-H3 having the sequence as set forth in SEQ ID NO: 5 or a variant thereof; and/or, the following three light chain CDRs defined by the Chothia numbering system: CDR-L1 having the sequence as set forth in SEQ ID NO: 6 or a variant thereof; CDR-L2 having the sequence as set forth in SEQ ID NO: 7 or a variant thereof; CDR-L3 having the sequence as set forth in SEQ ID NO: 8 or a variant thereof;
or
(2a) the following three heavy chain CDRs defined by the Kabat numbering system: CDR-H1 having the sequence as set forth in SEQ ID NO: 18 or a variant thereof; CDR-H2 having the sequence as set forth in SEQ ID NO: 19 or a variant thereof; CDR-H3 having the sequence as set forth in SEQ ID NO: 20 or a variant thereof; and/or, the following three light chain CDRs defined by the Kabat numbering system: CDR-L1 having the sequence as set forth in SEQ ID NO: 21 or a variant thereof; CDR-L2 having the sequence as set forth in SEQ ID NO: 22 or a variant thereof; CDR-L3 having the sequence as set forth in SEQ ID NO: 23 or a variant thereof;
or
(2b) the following three heavy chain CDRs defined by the IMGT numbering system: CDR-H1 having the sequence as set forth in SEQ ID NO: 24 or a variant thereof; CDR-H2 having the sequence as set forth in SEQ ID NO: 25 or a variant thereof; CDR-H3 having the sequence as set forth in SEQ ID NO: 26 or a variant thereof; and/or, the following three light chain CDRs defined by the IMGT numbering system: CDR-L1 having the sequence as set forth in SEQ ID NO: 27 or a variant thereof; CDR-L2 having the sequence as set forth in SEQ ID NO: 28 or a variant thereof; CDR-L3 having the sequence as set forth in SEQ ID NO: 23 or a variant thereof;
or
(2c) the following three heavy chain CDRs defined by the Chothia numbering system: CDR-H1 having the sequence as set forth in SEQ ID NO: 29 or a variant thereof; CDR-H2 having the sequence as set forth in SEQ ID NO: 30 or a variant thereof; CDR-H3 having the sequence as set forth in SEQ ID NO: 20 or a variant thereof; and/or, the following three light chain CDRs defined by the Chothia numbering system: CDR-L1 having the sequence as set forth in SEQ ID NO: 21 or a variant thereof; CDR-L2 having the sequence as set forth in SEQ ID NO: 22 or a variant thereof; CDR-L3 having the sequence as set forth in SEQ ID NO: 23 or a variant thereof;
or
(3a) the following three heavy chain CDRs defined by the Kabat numbering system: CDR-H1 having the sequence as set forth in SEQ ID NO: 33 or a variant thereof; CDR-H2 having the sequence as set forth in SEQ ID NO: 34 or a variant thereof; CDR-H3 having the sequence as set forth in SEQ ID NO: 35 or a variant thereof and/or, the following three light chain CDRs defined by the Kabat numbering system: CDR-L1 having the sequence as set forth in SEQ ID NO: 36 or a variant thereof; CDR-L2 having the sequence as set forth in SEQ ID NO: 37 or a variant thereof; CDR-L3 having the sequence as set forth in SEQ ID NO: 38 or a variant thereof;
or
(3b) the following three heavy chain CDRs defined by the IMGT numbering system: CDR-H1 having the sequence as set forth in SEQ ID NO: 39 or a variant thereof; CDR-H2 having the sequence as set forth in SEQ ID NO: 40 or a variant thereof; CDR-H3 having the sequence as set forth in SEQ ID NO: 41 or a variant thereof; and/or, the following three light chain CDRs defined by the IMGT numbering system: CDR-L1 having the sequence as set forth in SEQ ID NO: 42 or a variant thereof; CDR-L2 having the sequence as set forth in SEQ ID NO: 43 or a variant thereof; CDR-L3 having the sequence as set forth in SEQ ID NO: 38 or a variant thereof;
or
(3c) the following three heavy chain CDRs defined by the Chothia numbering system: CDR-H1 having the sequence as set forth in SEQ ID NO: 44 or a variant thereof; CDR-H2 having the sequence as set forth in SEQ ID NO: 45 or a variant thereof; CDR-H3 having the sequence as set forth in SEQ ID NO: 35 or a variant thereof; and/or, the following three light chain CDRs defined by the Chothia numbering system: CDR-L1 having the sequence as set forth in SEQ ID NO: 36 or a variant thereof; CDR-L2 having the sequence as set forth in SEQ ID NO: 37 or a variant thereof; CDR-L3 having the sequence as set forth in SEQ ID NO: 38 or a variant thereof;
wherein the variant described in any one of (1a), (1b), (1c), (2a), (2b), (2c), (3a), (3b), (3c) has a substitution, deletion or addition of one or several amino acids (e.g., a substitution, deletion or addition of 1, 2 or 3 amino acids) as compared to the sequence from which it is derived; preferably, the substitution is a conservative substitution;
preferably, the antibody or antigen-binding fragment thereof further comprises framework regions (FRs) derived from human immunoglobulin;
preferably, the antibody or antigen-binding fragment thereof specifically binds to a human MSLN.

2. The antibody or antigen-binding fragment thereof according to claim 1, wherein the antibody or antigen-binding fragment thereof comprises:
(1) a VH comprising the sequence as set forth in SEQ ID NO: 1 or a variant thereof and/or a VL comprising the sequence as set forth in SEQ ID NO: 2 or a variant thereof;
(2) a VH comprising the sequence as set forth in SEQ ID NO: 16 or a variant thereof and/or a VL comprising the sequence as set forth in SEQ ID NO: 17 or a variant thereof; or
(3) a VH comprising the sequence as set forth in SEQ ID NO: 31 or a variant thereof and/or a VL comprising the sequence as set forth in SEQ ID NO: 32 or a variant thereof;
wherein the variant has a sequence identity of at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or has a substitution, deletion or addition of one or several amino acids (e.g., a substitution, deletion or addition of 1, 2, 3, 4 or 5 amino acids) as compared to the sequence from which it is derived; preferably, the substitution is a conservative substitution.

3-5. (canceled)

6. The antibody or antigen-binding fragment thereof according to claim 1, wherein the antibody or antigen-binding fragment thereof is selected from the group consisting of camelid Ig, IgNAR, Fab fragment, Fab′ fragment, F(ab)′₂ fragment, F(ab)′₃ fragment, single chain antibody (e.g., scFv, di-scFv or (scFv)₂), minibody, bifunctional antibody, trifunctional antibody, tetrafunctional antibody, disulfide-stabilized Fv protein (dsFv) and single domain antibody (sdAb, nanobody).

7. The antibody or antigen-binding fragment thereof according to claim 1, wherein the antibody or antigen-binding fragment thereof is a single chain antibody.

8. The antibody or antigen-binding fragment thereof according to claim 1, wherein the antibody or antigen-binding fragment thereof further comprises a heavy chain constant region (CH) and a light chain constant region (CL);
preferably, the heavy chain constant region is selected from the group consisting of IgG, IgM, IgE, IgD and IgA;
preferably, the light chain constant region is selected from κ or λ.

9. An isolated nucleic acid molecule, which comprises a nucleotide sequence encoding the antibody or antigen-binding fragment thereof according to claim 1.

10. A vector, which comprises an isolated nucleic acid molecule comprising a nucleotide sequence encoding the antibody or antigen-binding fragment thereof according to claim 1;
preferably, the vector is selected from the group consisting of DNA vector, RNA vector, plasmid, transposon vector, CRISPR/Cas9 vector or viral vector;
preferably, the vector is an expression vector;
preferably, the vector is an episomal vector;
preferably, the vector is a viral vector; more preferably, the viral vector is a lentiviral vector, adenoviral vector or retroviral vector.

11. A host cell, which comprises an isolated nucleic acid molecule comprising a nucleotide sequence encoding the antibody or antigen-binding fragment thereof according to claim 1, or a vector comprising the isolated nucleic acid molecule.

12. A method for preparing the antibody or antigen-binding fragment thereof according to claim 1, which comprises: culturing a host cell comprising a nucleotide sequence encoding the antibody or antigen-binding fragment thereof or a vector comprising the isolated nucleic acid molecule under conditions that allow the expression of the antibody or antigen-binding fragment thereof, and recovering the antibody or antigen-binding fragment thereof from a cultured host cell culture.

13. A chimeric antigen receptor (CAR) capable of specifically binding to MSLN, which comprises an antigen-binding domain, a spacer domain, a transmembrane domain, and an intracellular signaling domain, wherein the antigen-binding domain comprises the antibody or antigen-binding fragment thereof according to claim 1;
preferably, the antigen-binding domain comprises the antibody or antigen-binding fragment thereof as a first binding domain, and further comprises a second binding domain that does not bind to MSLN; more preferably, the second binding domain binds to an antigen that is selected from the group consisting of: CD19, GPC3, PSMA, MUC1, EGFR, HER2, CD276, GD2, BCMA, CD33 or Claudin18.2;
preferably, the antibody or antigen-binding fragment thereof is a single chain antibody, such as scFv, di-scFv or (scFv)₂;
preferably, the VH and VL of the antibody or antigen-binding fragment thereof are linked via a linker; preferably, the linker comprises one or several (e.g., 1, 2 or 3) sequences shown as (GₘS)ₙ, wherein m is an integer selected from 1 to 6, and n is an integer selected from 1 to 6; preferably, m is 3, 4 or 5; preferably, n is 1 or 2; more preferably, the linker has the sequence as set forth in SEQ ID NO:52.

14. The chimeric antigen receptor according to claim 13, wherein the antigen-binding domain comprises an amino acid sequence selected from the group consisting of: (1) an amino acid sequence as set forth in any one of SEQ ID NOs: 54, 56, 58; (2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence as set forth in any one of SEQ ID NOs: 54, 56, 58; or (3) a sequence having a substitution, deletion or addition of one or more amino acids (e.g., a substitution, deletion or addition of 1, 2, 3, 4, 5, 6, 7, 8, 9 or 10 amino acids) as compared to the amino acid sequence as set forth in any one of SEQ ID NOs: 54, 56, 58; preferably, the substitution is a conservative substitution.

15-18. (canceled)

19. The chimeric antigen receptor according to claim 13, wherein the chimeric antigen receptor comprises a signal peptide, an antigen-binding domain, a spacer domain, a transmembrane domain, an intracellular signaling domain in order from its N-terminal to C-terminal; wherein
the signal peptide comprises a heavy chain signal peptide of IgG1 or a CD8α signal peptide (e.g., a signal peptide having the sequence as set forth in SEQ ID NO: 60);
the spacer domain comprises a hinge region of CD8 (e.g., CD8α) (e.g., a hinge region having the sequence as set forth in SEQ ID NO: 62);
the transmembrane domain comprises a transmembrane region of CD8 (e.g., CD8α) (e.g., a transmembrane region having the sequence as set forth in SEQ ID NO: 64);
preferably, the intracellular signaling domain comprises a primary signaling domain and a costimulatory signaling domain, wherein the primary signaling domain comprises an intracellular signaling domain of CD3 (e.g., a sequence as set forth in SEQ ID NO: 68), the costimulatory signaling domain comprises an intracellular signaling domain of CD137 (4-1BB) (e.g., a sequence as set forth in SEQ ID NO: 66); more preferably, the intracellular signaling domain of the chimeric antigen receptor has the sequence as set forth in SEQ ID NO: 70.

20. An isolated nucleic acid molecule, which comprises a nucleotide sequence encoding the chimeric antigen receptor according to claim 13.

21. A nucleic acid construct, which comprises:
(1) a first nucleic acid sequence encoding the chimeric antigen receptor according to claim 13; and
(2) a second nucleic acid sequence encoding an additional biologically active molecule;
preferably, the additional biologically active molecule encoded by the second nucleotide sequence is one or more selected from the following components: immune checkpoint inhibitor (e.g., anti-PD-1, PD-L1, CTLA-4, or LAG-3 antibody or antigen-binding fragment thereof), cytokine (e.g., IL-15, IL-7, IL-12, IL-18, or IL-21), or membrane-bound polypeptide (e.g., mIL-15, mIL-7, mIL-12, mIL-18, or mIL-21);
preferably, the additional biologically active molecule encoded by the second nucleotide sequence further comprises a signal peptide-2 at its N-terminal; preferably, the signal peptide-2 is different from the signal peptide contained in the chimeric antigen receptor encoded by the first nucleic acid sequence; preferably, the signal peptide-2 at the N-terminal of the additional biologically active molecule is an IL2 signal peptide (e.g., the one as set forth in SEQ ID NO: 74).

22. The nucleic acid construct according to claim 21, wherein the first nucleic acid sequence and the second nucleic acid sequence are linked via a nucleotide sequence encoding a self-cleaving peptide (e.g., P2A, E2A, F2A, T2A or any combination thereof);
preferably, the self-cleaving peptide is P2A (e.g., the one as set forth in SEQ ID NO: 72).

23. The nucleic acid construct according to claim 21, wherein the additional biologically active molecule is selected from an immune checkpoint inhibitor that is an anti-PD-1 or PD-L1 antibody or an antigen-binding fragment thereof;
preferably, the anti-PD-1 or PD-L1 antibody or antigen-binding fragment thereof comprises a heavy chain variable region and/or a light chain variable region of any one of the following groups: (1) a heavy chain variable region and/or light chain variable region of Nivolumab or a variant thereof, (2) a heavy chain variable region and/or light chain variable region of Pembrolizumab or a variant thereof, (3) a heavy chain variable region and/or light chain variable region of Atezolizumab or a variant thereof, (4) a heavy chain variable region and/or light chain variable region of Durvalumab or a variant thereof, (5) a heavy chain variable region and/or light chain variable region of Avelumab or a variant thereof, (6) a VH having the sequence as set forth in SEQ ID NO: 79 or a variant thereof and/or a VL having the sequence as set forth in SEQ ID NO: 80 or a variant thereof; wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or has a substitution, deletion or addition of one or several amino acids (e.g., a substitution, deletion or addition of 1, 2, 3, 4, 5, 6, 7, 8, 9 or 10 amino acids) as compared to the sequence from which it is derived; preferably, the substitution is a conservative substitution;
preferably, the anti-PD-1 or PD-L1 antibody or antigen-binding fragment thereof is a single-chain antibody (e.g., scFv);
preferably, the additional biologically active molecule comprises an anti-PD-1 single chain antibody, and the anti-PD-1 single chain antibody comprises an amino acid sequence selected from the group consisting of: (1) an amino acid sequence as set forth in SEQ ID NO: 77; (2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence as set forth in SEQ ID NO: 77; (3) a sequence having a substitution, deletion or addition of one or several amino acids (e.g., a substitution, deletion or addition of 1, 2, 3, 4, 5, 6, 7, 8, 9 or 10 amino acids) as compared to the amino acid sequence as set forth in SEQ ID NO: 77; preferably, the substitution is a conservative substitution;
preferably, the nucleic acid construct comprises in order from its 5′ end to its 3′ end: the first nucleic acid sequence, a nucleotide sequence encoding a self-cleavage peptide, a nucleotide sequence encoding a signal peptide-2, a nucleotide sequence encoding an immune checkpoint inhibitor; preferably, the nucleic acid construct comprises a nucleotide sequence selected from the group consisting of: (1) a nucleotide sequence as set forth in SEQ ID NO: 85 or a degenerate variant thereof; (2) a sequence substantially identical to the sequence as described in (1), for example, a sequence having a sequence identity of at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the sequence as described in (1).

24. The nucleic acid construct according to claim 21, wherein the additional biologically active molecule is selected from a membrane-bound polypeptide that is mIL-15;
preferably, the membrane-bound polypeptide mIL-15 comprises an amino acid sequence selected from the group consisting of: (1) a sequence as set forth in SEQ ID NO: 81; (2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence as set forth in SEQ ID NO: 81; (3) a sequence having a substitution, deletion or addition of one or several amino acids (e.g., a substitution, deletion or addition of 1, 2, 3, 4, 5, 6, 7, 8, 9, or 10 amino acids) as compared to the amino acid sequence as set forth in SEQ ID NO: 81; preferably, the substitution is a conservative substitution;
preferably, the nucleic acid construct comprises in order from its 5′ end to its 3′ end: the first nucleic acid sequence, a nucleotide sequence encoding a self-cleaving peptide, a nucleotide sequence encoding a membrane chimeric polypeptide; preferably, the nucleic acid construct comprises a nucleotide sequence selected from the group consisting of: (1) a nucleotide sequence as set forth in SEQ ID NO: 86 or a degenerate variant thereof; (2) a sequence substantially identical to the sequence as described in (1), for example, a sequence having a sequence identity of at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the sequence as described in (1).

25. The nucleic acid construct according to claim 21, wherein the additional biologically active molecule encoded by the second nucleic acid sequence comprises at least two components selected from the group consisting of: immune checkpoint inhibitor (e.g., anti-PD-1, PD-L1, CTLA-4, or LAG-3 antibody or antigen-binding fragment thereof), cytokine (e.g., IL-15, IL-7, IL-12, IL-18, or IL-21), or membrane-bound polypeptide (e.g., mIL-15, mIL-7, mIL-12, mIL-18, or mIL-21);
preferably, the nucleotide sequences encoding the at least two components contained in the second nucleic acid sequence are linked to each other through a nucleotide sequence encoding a self-cleaving peptide (e.g., P2A, E2A, F2A, T2A or any combination thereof); preferably, the self-cleaving peptide is P2A (e.g., the one as set forth in SEQ ID NO: 72);
preferably, the additional biologically active molecule encoded by the second nucleic acid sequence comprises: (i) an anti-PD-1 antibody or antigen-binding fragment thereof (e.g., scFv) and (ii) mIL-15;
preferably, the nucleic acid construct comprises in order from the 5′ end to the 3′ end: the first nucleic acid sequence, a nucleotide sequence encoding a self-cleaving peptide, a nucleotide sequence encoding a signal peptide-2, a nucleic acid encoding an anti-PD-1 antibody or antigen-binding fragment thereof, a nucleotide sequence encoding a self-cleaving peptide, a nucleotide sequence encoding mIL-15; preferably, the nucleic acid construct comprises a nucleotide sequence selected from the group consisting of: (1) a nucleotide sequence as set forth in SEQ ID NO: 87 or a degenerate variant thereof; (2) a sequence substantially identical to the sequence described in (1), for example, a sequence having a sequence identity of at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the sequence described in (1).

26. A vector, which comprises (i) an isolated nucleic acid molecule comprising a nucleotide sequence encoding the chimeric antigen receptor according to claim 13, or (ii) a nucleic acid construct comprising a first nucleic acid sequence encoding the chimeric antigen receptor and a second nucleic acid sequence encoding an additional biologically active molecule;
preferably, the vector is selected from the group consisting of DNA vector, RNA vector, plasmid, transposon vector, CRISPR/Cas9 vector, or viral vector;
preferably, the vector is an expression vector;
preferably, the vector is an episomal vector;
preferably, the vector is a viral vector; more preferably, the viral vector is a lentiviral vector, adenoviral vector or retroviral vector.

27. An engineered immune cell, which expresses the chimeric antigen receptor (CAR) according to claim 13; preferably, the engineered immune cell comprises an isolated nucleic acid molecule comprising a nucleotide sequence encoding the chimeric antigen receptor or a vector comprising the isolated nucleic acid molecule.

28. The engineered immune cell according to claim 27, which further expresses an additional biologically active molecule, wherein the additional biologically active molecule is one or more selected from the following components: an antibody or antigen-binding fragment thereof that specifically binds to an immune checkpoint (e.g., anti-PD-1, PD-L1, CTLA-4, or LAG-3 antibody or antigen-binding fragment thereof), cytokine (e.g., IL-15, IL-7, IL-12, IL-18, IL-21), or membrane-bound polypeptide (e.g., mIL-15, mIL-7, mIL-12, mIL-18, mIL-21);
preferably, the engineered immune cell comprises a nucleic acid construct or a vector comprising the nucleic acid construct, wherein the nucleic acid construct comprises: (1) a first nucleic acid sequence encoding the chimeric antigen receptor; and (2) a second nucleic acid sequence encoding the additional biologically active molecule;
preferably, the first nucleic acid sequence and the second nucleic acid sequence are linked via a nucleotide sequence encoding a self-cleaving peptide (e.g., P2A, E2A, F2A, T2A or any combination thereof);
preferably, the self-cleaving peptide is P2A (e.g., the one as set forth in SEQ ID NO: 72).

29. The engineered immune cell according to claim 28, wherein the additional biologically active molecule is an anti-PD-1 or PD-L1 antibody or antigen-binding fragment thereof;
preferably, the engineered immune cell comprises a nucleic acid construct or a vector comprising the nucleic acid construct, wherein the nucleic acid construct comprises: (1) a first nucleic acid sequence encoding the chimeric antigen receptor; and (2) a second nucleic acid sequence encoding the anti-PD-1 or PD-L1 antibody or antigen-binding fragment thereof;
preferably, the anti-PD-1 or PD-L1 antibody or antigen-binding fragment thereof comprises a heavy chain variable region and/or a light chain variable region of any one of the following groups: (1) a heavy chain variable region and/or light chain variable region of Nivolumab or a variant thereof, (2) a heavy chain variable region and/or light chain variable region of Pembrolizumab or a variant thereof, (3) a heavy chain variable region and/or light chain variable region of Atezolizumab or a variant thereof, (4) a heavy chain variable region and/or light chain variable region of Durvalumab or a variant thereof, (5) a heavy chain variable region and/or light chain variable region of Avelumab or a variant thereof, (6) a VH having the sequence as set forth in SEQ ID NO: 79 or a variant thereof and/or a VL having the sequence as set forth in SEQ ID NO: 80 or a variant thereof; wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or has a substitution, deletion or addition of one or several amino acids (e.g., a substitution, deletion or addition of 1, 2, 3, 4, 5, 6, 7, 8, 9 or 10 amino acids) as compared to the sequence from which it is derived; preferably, the substitution is a conservative substitution;
preferably, the anti-PD-1 or PD-L1 antibody or antigen-binding fragment thereof is a single-chain antibody (e.g., scFv);
preferably, the anti-PD-1 single chain antibody comprises an amino acid sequence selected from the group consisting of: (1) an amino acid sequence as set forth in SEQ ID NO: 77; (2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence as set forth in SEQ ID NO: 77; (3) a sequence having a substitution, deletion or addition of one or several amino acids (e.g., a substitution, deletion or addition of 1, 2, 3, 4, 5, 6, 7, 8, 9 or 10 amino acids) as compared to the amino acid sequence as set forth in SEQ ID NO: 77; preferably, the substitution is a conservative substitution;
preferably, the nucleic acid construct comprises in order from its 5′ end to its 3′ end: the first nucleic acid sequence, a nucleotide sequence encoding a self-cleavage peptide, a nucleotide sequence encoding a signal peptide-2, a nucleotide sequence encoding the anti-PD-1 or PD-L1 antibody or antigen-binding fragment thereof; preferably, the nucleic acid construct comprises a nucleotide sequence selected from the group consisting of: (1) a nucleotide sequence as set forth in SEQ ID NO: 85 or a degenerate variant thereof; (2) a sequence substantially identical to the sequence as described in (1), for example, a sequence having a sequence identity of at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the sequence as described in (1).

30. The engineered immune cell according to claim 28, wherein the additional biologically active molecule is mIL-15;
preferably, the engineered immune cell comprises a nucleic acid construct or a vector comprising the nucleic acid construct, wherein the nucleic acid construct comprises: (1) a first nucleic acid sequence encoding the chimeric antigen receptor; and (2) a second nucleic acid sequence encoding the mIL-15;
preferably, the mIL-15 comprises an amino acid sequence selected from the group consisting of: (1) a sequence as set forth in SEQ ID NO: 81; (2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence as set forth in SEQ ID NO: 81; (3) a sequence having a substitution, deletion or addition of one or several amino acids (e.g., a substitution, deletion or addition of 1, 2, 3, 4, 5, 6, 7, 8, 9, or 10 amino acids) as compared to the amino acid sequence as set forth in SEQ ID NO: 81; preferably, the substitution is a conservative substitution;
preferably, the nucleic acid construct comprises in order from its 5′ end to its 3′ end: the first nucleic acid sequence, a nucleotide sequence encoding a self-cleaving peptide, a nucleotide sequence encoding the mIL-15; preferably, the nucleic acid construct comprises a nucleotide sequence selected from the group consisting of: (1) a nucleotide sequence as set forth in SEQ ID NO: 86 or a degenerate variant thereof; (2) a sequence substantially identical to the sequence as described in (1), for example, a sequence having a sequence identity of at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the sequence as described in (1).

31. The engineered immune cell according to claim 28, wherein the additional biologically active molecule comprises at least two components selected from the group consisting of: immune checkpoint inhibitor (e.g., anti-PD-1, PD-L1, CTLA-4, or LAG-3 antibody or antigen-binding fragment thereof), cytokine (e.g., IL-15, IL-7, IL-12, IL-18, or IL-21), or membrane-bound polypeptide (e.g., mIL-15, mIL-7, mIL-12, mIL-18, or mIL-21);
preferably, the engineered immune cell comprises a nucleic acid construct or a vector comprising the nucleic acid construct, wherein the nucleic acid construct comprises: (1) a first nucleic acid sequence encoding the chimeric antigen receptor; and (2) a second nucleic acid sequence encoding the additional biologically active molecule;
preferably, the nucleotide sequences encoding the at least two components contained in the second nucleic acid sequence are linked to each other through a nucleotide sequence encoding a self-cleaving peptide (e.g., P2A, E2A, F2A, T2A or any combination thereof); preferably, the self-cleaving peptide is P2A (e.g., the one as set forth in SEQ ID NO: 72);
preferably, the additional biologically active molecule encoded by the second nucleic acid sequence comprises: (i) an anti-PD-1 antibody or antigen-binding fragment thereof (e.g., scFv) and (ii) mIL-15;
preferably, the nucleic acid construct comprises in order from the 5′ end to the 3′ end: the first nucleic acid sequence, a nucleotide sequence encoding a self-cleaving peptide, a nucleotide sequence encoding a signal peptide-2, a nucleic acid encoding an anti-PD-1 antibody or antigen-binding fragment thereof, a nucleotide sequence encoding a self-cleaving peptide, a nucleotide sequence encoding mIL-15; preferably, the nucleic acid construct comprises a nucleotide sequence selected from the group consisting of: (1) a nucleotide sequence as set forth in SEQ ID NO: 87 or a degenerate variant thereof; (2) a sequence substantially identical to the sequence described in (1), for example, a sequence having a sequence identity of at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the sequence described in (1).

32. The engineered immune cell according to claim 27 wherein the immune cell is derived from T lymphocyte, NK cell, monocyte, macrophage or dendritic cell and any combination thereof; preferably, the immune cell is obtained from a patient; alternatively, the immune cell is obtained from a healthy donor; preferably, the immune cell is derived from T lymphocyte or NK cell.

33. The engineered immune cell according to claim 27 wherein the immune cell further expresses a CAR that is not specific for MSLN; preferably, the CAR that is not specific for MSLN has a specificity for a target selected from the group consisting of: CD19, GPC3, PSMA, MUC1, EGFR, HER2, CD276, GD2, BCMA, CD33 or Claudin18.2.

34. The engineered immune cell according to claim 27 wherein the transcription or expression of one or two target genes selected from a gene involved in the immune exclusion of the engineered immune cell (e.g., TRAC, TRBC, B2M, HLA-A, HLA-B, or HLA-C) and a gene of immune co-inhibitory pathway or signaling molecule (e.g., PD-1, CTLA-4 or LAG-3) is inhibited; preferably, the method by which the transcription or expression of the target genes is inhibited is selected from the group consisting of gene knockout (e.g., CRISPR, CRISPR/Cas9), homologous recombination, and interfering RNA.

35. A method for preparing an engineered immune cell, which comprises: (1) providing an immune cell from a patient or healthy donor; (2) introducing one of the following into the immune cell described in step (1): (i) an isolated nucleic acid molecule comprising a nucleotide sequence encoding the chimeric antigen receptor according to claim 13; or (ii) a nucleic acid construct comprising a first nucleic acid sequence encoding the chimeric antigen receptor and a second nucleic acid sequence encoding an additional biologically active molecule; or (iii) a vector comprising the isolated nucleic acid molecule of (i) or the nucleic acid construct of (ii);
preferably, in step (1), the immune cell is subjected to pretreatment, and the pretreatment comprises sorting, activation and/or proliferation of the immune cell; more preferably, the pretreatment comprises contacting the immune cell with an anti-CD3 antibody and an anti-CD28 antibody, thereby stimulating the immune cell and inducing its proliferation, thereby generating a pretreated immune cell;
preferably, in step (2), the nucleic acid molecule or vector is introduced into the immune cell by viral infection;
preferably, in step (2), the nucleic acid molecule or vector is introduced into the immune cell by means of non-viral vector transfection, such as calcium phosphate transfection, DEAE-dextran-mediated transfection, microinjection, transposon vector system, CRISPR/Cas9 vector, TALEN method, ZFN method or electroporation method;
preferably, a step of expanding the immune cell obtained in step (2) is further comprised after step (2).

36. An immune cell composition, comprising an engineered immune cell which comprises: (i) an isolated nucleic acid molecule comprising a nucleotide sequence encoding the chimeric antigen receptor according to claim 13 or (ii) a nucleic acid construct comprising a first nucleic acid sequence encoding the chimeric antigen receptor and a second nucleic acid sequence encoding an additional biologically active molecule or (iii) a vector comprising the isolated nucleic acid molecule of (i) or the nucleic acid construct of (ii); alternatively, the composition further comprises an unmodified and/or unsuccessfully engineered immune cell; preferably, the number of the engineered immune cell accounts for 10% to 100%, more preferably 40% to 80% of the total number of cells in the immune cell composition.

37. A kit, wherein the kit comprises: (i) the antibody or antigen-binding fragment thereof according to claim 1, or (ii) an isolated nucleic acid molecule comprising a nucleotide sequence encoding the antibody or antigen-binding fragment thereof, or (iii) a vector comprising the isolated nucleic acid molecule, or (iv) a host cell comprising the isolated nucleic acid molecule of (ii) or the vector of (iii).

38. A pharmaceutical composition, which comprises a pharmaceutically acceptable carrier and/or excipient and one of the following: (i) the antibody or antigen-binding fragment thereof according to claim 1, or (ii) an isolated nucleic acid molecule comprising a nucleotide sequence encoding the antibody or antigen-binding fragment thereof, or (iii) a vector comprising the isolated nucleic acid molecule, or (iv) a host cell comprising the isolated nucleic acid molecule of (ii) or the vector of (iii);
preferably, the pharmaceutical composition further comprises an additional pharmaceutically active agent, such as a drug with anti-tumor activity; preferably, the additional pharmaceutically active agent comprises anti-PD1 antibody, anti-PD-L1 antibody, anti-CTLA-4 antibody, pemetrexed, cisplatin, paclitaxel, gemcitabine, capecitabine, or FOLFIRINOX;
optionally, the additional pharmaceutically active agent is administered simultaneously, separately or sequentially.

39. (canceled)

40. A method for preventing and/or treating a disease associated with mesothelin expression in a subject (such as a human), the method comprising administering to a subject in need thereof an effective amount of (i) the antibody or antigen-binding fragment thereof according to claim 1, or (ii) an isolated nucleic acid molecule encoding the antibody or antigen-binding fragment thereof, or (iii) a vector comprising the isolated nucleic acid molecule, or (iv) a host cell comprising the isolated nucleic acid molecule or vector, or (v) a pharmaceutical composition comprising any one of (i)-(iv);
preferably, the disease associated with mesothelin expression is selected from a proliferative disease, such as a tumor, or a non-tumor-related indication associated with mesothelin expression;
preferably, the tumor is an MSLN-positive tumor;
preferably, the tumor is selected from a solid tumor; preferably, the solid tumor is selected from the group consisting of malignant pleural mesothelioma, pancreatic cancer, lung cancer (e.g., lung squamous carcinoma), breast cancer, ovarian cancer (e.g., ovarian epithelial cancer);
preferably, the method further comprises administering to the subject a second therapy, and the second therapy is selected from the group consisting of surgery, chemotherapy, radiotherapy, immunotherapy, gene therapy, DNA therapy, RNA therapy, nanotherapy, viral therapy, adjuvant therapy and any combination thereof.

41. A method for preventing and/or treating a disease associated with mesothelin expression in a subject (such as a human),
the method comprises the steps of: (1) providing an immune cell required by the subject; (2) introducing one of the following into the immune cell described in step (1) (i) an isolated nucleic acid molecule comprising a nucleotide sequence encoding the chimeric antigen receptor according to claim 13, or (ii) a nucleic acid construct comprising a first nucleic acid sequence encoding the chimeric antigen receptor and a second nucleic acid sequence encoding an additional biologically active molecule, or (iii) a vector comprising the isolated nucleic acid molecule or nucleic acid construct; (3) administering the immune cell obtained in step (2) to the subject;
optionally, in step (3), the total dose of the immune cell comprises 1×10⁷ to 10×10⁸ CAR-positive cells;
preferably, in step (3), the total dose of the immune cell is administered to the subject in divided doses.

42. The chimeric antigen receptor according to claim 13, characterized by one or more of the following:
(i) the transmembrane domain is a transmembrane region selected from the following proteins: α, β or ζ chain of T cell receptor, CD28, CD45, CD3ε, CD3ζ, CD4, CD5, CD8α, CD9, CD16, CD22, CD33, CD37, CD64, CD80, CD86, CD134, CD137, CD152, CD154 and PD-1; preferably, the transmembrane domain is a transmembrane region selected from the following proteins: CD8α, CD4, PD-1, CD152 and CD154; preferably, the transmembrane domain comprises a CD8α transmembrane domain having the sequence as set forth in SEQ ID NO: 64;
(ii) the spacer domain is located between the antigen-binding domain and the transmembrane domain, and the spacer domain is selected from the group consisting of a hinge domain and/or CH2 and CH3 regions of an immunoglobulin (e.g., IgG1 or IgG4); preferably, the hinge domain comprises a hinge region of CD8α, PD-1, CD152 or CD154; more preferably, the hinge domain comprises a CD8α hinge region having the sequence as set forth in SEQ ID NO: 62;
(iii) the intracellular signaling domain comprises a primary signaling domain and/or a costimulatory signaling domain; preferably, the primary signaling domain comprises an immunoreceptor tyrosine activation motif (ITAM); preferably, the primary signaling domain comprises an intracellular signaling domain of a protein selected from the group consisting of: CD3ζ, FcRγ, FcRβ, CD3γ, CD3δ, CD3ε, CDS, CD22, CD79a, CD79b or CD66d; more preferably, the primary signaling domain comprises a CD3ζ intracellular signaling domain having the sequence as set forth in SEQ ID NO: 68; preferably, the costimulatory signaling domain comprises an intracellular signaling domain of a protein selected from the group consisting of: CARD11, CD2, CD7, CD27, CD28, CD30, CD134(OX40), CD137(4-1BB), CD150(SLAMF1), CD270(HVEM), CD278(ICOS) or DAP10; preferably, the costimulatory signaling domain is selected from the group consisting of an intracellular signaling domain of CD28 or an intracellular signaling domain of CD137(4-1BB) or a combination of fragments thereof; more preferably, the costimulatory signaling domain comprises an CD137(4-1BB) intracellular signaling domain having the sequence as set forth in SEQ ID NO: 66; more preferably, the intracellular signaling domain sequence comprises the sequence as set forth in SEQ ID NO: 70;
(iv) the chimeric antigen receptor further comprises a signal peptide at its N-terminal; the signal peptide comprises a heavy chain signal peptide (e.g., a heavy chain signal peptide of IgG1), a granulocyte-macrophage colony stimulating factor receptor 2 (GM-CSFR2) signal peptide, an IL2 signal peptide, or a CD8α signal peptide; more preferably, the signal peptide comprises the sequence as set forth in SEQ ID NO: 60.

43. A kit, wherein the kit comprises: (i) an isolated nucleic acid molecule comprising a nucleotide sequence encoding the chimeric antigen receptor according to claim 13; or (ii) a nucleic acid construct comprising a first nucleic acid sequence encoding the chimeric antigen receptor and a second nucleic acid sequence encoding an additional biologically active molecule; or (iii) a vector comprising the isolated nucleic acid molecule of (i) or the nucleic acid construct of (ii).

44. A pharmaceutical composition, which comprises a pharmaceutically acceptable carrier and/or excipient and one of the following: (i) an isolated nucleic acid molecule comprising a nucleotide sequence encoding the chimeric antigen receptor according to claim 13; or (ii) a nucleic acid construct comprising a first nucleic acid sequence encoding the chimeric antigen receptor and a second nucleic acid sequence encoding an additional biologically active molecule; or (iii) a vector comprising the isolated nucleic acid molecule of (i) or the nucleic acid construct of (ii); or (iv) an engineered immune cell comprising the isolated nucleic acid molecule of (i) or the nucleic acid construct of (ii) or the vector of (iii); or (v) an immune cell composition comprising the engineered immune cell of (iv);
preferably, the pharmaceutical composition further comprises an additional pharmaceutically active agent, such as a drug with anti-tumor activity; preferably, the additional pharmaceutically active agent comprises anti-PD1 antibody, anti-PD-L1 antibody, anti-CTLA-4 antibody, pemetrexed, cisplatin, paclitaxel, gemcitabine, capecitabine, or FOLFIRINOX;
optionally, the additional pharmaceutically active agent is administered simultaneously, separately or sequentially.

45. A method for preventing and/or treating a disease associated with mesothelin expression in a subject (such as a human), the method comprising administering to a subject in need thereof an effective amount of: (i) an isolated nucleic acid molecule comprising a nucleotide sequence encoding the chimeric antigen receptor according to claim 13; or (ii) a nucleic acid construct comprising a first nucleic acid sequence encoding the chimeric antigen receptor and a second nucleic acid sequence encoding an additional biologically active molecule; or (iii) a vector comprising the isolated nucleic acid molecule of (i) or the nucleic acid construct of (ii); (iv) an engineered immune cell comprising the isolated nucleic acid molecule of (i) or the nucleic acid construct of (ii) or the vector of (iii); or (v) an immune cell composition comprising the engineered immune cell of (iv); or (vi) a pharmaceutical composition comprising any one of (i)-(v);
preferably, the disease associated with mesothelin expression is selected from a proliferative disease, such as a tumor, or a non-tumor-related indication associated with mesothelin expression;
preferably, the tumor is an MSLN-positive tumor;
preferably, the tumor is selected from a solid tumor; preferably, the solid tumor is selected from the group consisting of malignant pleural mesothelioma, pancreatic cancer, lung cancer (e.g., lung squamous carcinoma), breast cancer, ovarian cancer (e.g., ovarian epithelial cancer);
preferably, the method further comprises administering to the subject a second therapy, and the second therapy is selected from the group consisting of surgery, chemotherapy, radiotherapy, immunotherapy, gene therapy, DNA therapy, RNA therapy, nanotherapy, viral therapy, adjuvant therapy and any combination thereof.

46. The antibody or antigen-binding fragment thereof according to claim 7, wherein the antibody or antigen-binding fragment thereof is a single chain antibody;
and the single chain antibody comprises from its N-terminal to its C-terminal:
(1) a VH comprising the sequence as set forth in SEQ ID NO: 1 or a variant thereof—linker—a VL comprising the sequence as set forth in SEQ ID NO: 2 or a variant thereof;
(2) a VH comprising the sequence as set forth in SEQ ID NO: 16 or a variant thereof—linker—a VL comprising the sequence as set forth in SEQ ID NO: 17 or a variant thereof;
(3) a VH comprising the sequence as set forth in SEQ ID NO: 31 or a variant thereof—linker—a VL comprising the sequence as set forth in SEQ ID NO: 32 or a variant thereof;
(4) a VL comprising the sequence as set forth in SEQ ID NO: 2 or a variant thereof—linker—a VH comprising the sequence as set forth in SEQ ID NO: 1 or a variant thereof;
(5) a VL comprising the sequence as set forth in SEQ ID NO: 17 or a variant thereof—linker—a VH comprising the sequence as set forth in SEQ ID NO: 16 or a variant thereof; or
(6) a VL comprising the sequence as set forth in SEQ ID NO: 32 or a variant thereof—linker—a VH comprising the sequence as set forth in SEQ ID NO: 31 or a variant thereof;
wherein, the variant has a sequence identity of at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or has a substitution, deletion or addition of one or several amino acids (e.g., a substitution, deletion or addition of 1, 2, 3, 4 or 5 amino acids) as compared to the sequence from which it is derived; preferably, the substitution is a conservative substitution.

47. The antibody or antigen-binding fragment thereof according to claim 7, wherein the antibody or antigen-binding fragment thereof is a single chain antibody,
the VH and VL of the single chain antibody are linked via a linker; preferably, the linker is a polypeptide; preferably, the linker comprises one or several (e.g., 1, 2 or 3) sequences shown as (GmS)n, wherein m is an integer selected from 1 to 6, and n is an integer selected from 1 to 6; preferably, m is 3, 4 or 5; preferably, n is 1 or 2; more preferably, the linker has the sequence as set forth in SEQ ID NO: 52.

48. The antibody or antigen-binding fragment thereof according to claim 7, wherein the antibody or antigen-binding fragment thereof is a single chain antibody,
the single chain antibody comprises an amino acid sequence selected from the group consisting of: (1) an amino acid sequence as set forth in any one of SEQ ID NOs: 54, 56, 58; (2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence as set forth in any one of SEQ ID NOs: 54, 56, 58; or (3) a sequence having a substitution, deletion or addition of one or several amino acids (e.g., a substitution, deletion or addition of 1, 2, 3, 4, 5, 6, 7, 8, 9, or 10 amino acids) as compared to the amino acid sequence as set forth in any one of SEQ ID NOs: 54, 56, 58; preferably, the substitution is a conservative substitution.

49. An isolated nucleic acid molecule, which comprises a nucleotide sequence encoding the antibody or antigen-binding fragment thereof according to claim 9;
the isolated nucleic acid molecule comprises a nucleotide sequence selected from the group consisting of: (1) a nucleotide sequence as set forth in any one of SEQ ID NOs: 55, 57 and 59; (2) a sequence having a sequence identity of at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the nucleotide sequence as set forth in any one of SEQ ID NOs: 55, 57 and 59.

50. The chimeric antigen receptor according to claim 19, wherein the chimeric antigen receptor comprises a signal peptide, an antigen-binding domain, a spacer domain, a transmembrane domain, an intracellular signaling domain in order from its N-terminal to C-terminal; wherein:
the chimeric antigen receptor comprises an amino acid sequence selected from the group consisting of: (1) an amino acid sequence as set forth in any one of SEQ ID NOs: 83, 88, and 90; (2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence as set forth in any one of SEQ ID NOs: 83, 88, and 90; or, (3) a sequence having a substitution, deletion or addition of one or several amino acids (e.g., a substitution, deletion or addition of 1, 2, 3, 4, 5, 6, 7, 8, 9 or 10 amino acids) as compared to the amino acid sequence as set forth in any one of SEQ ID NOs: 83, 88, and 90; preferably, the substitution is a conservative substitution.

51. An isolated nucleic acid molecule, which comprises a nucleotide sequence encoding the chimeric antigen receptor according to claim 20;
the isolated nucleic acid molecule comprises a nucleotide sequence selected from the group consisting of: (1) a sequence as set forth in any one of SEQ ID NOs: 84, 89, 91 or a degenerate variant thereof; (2) a sequence substantially identical to the sequence of any one of (1) (e.g., a sequence having a sequence identity of at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% %, at least 99%, or 100% as compared to the sequence of any one of (1), or, a sequence having a substitution of one or more nucleotides as compared to the sequence of any one of (1)).
